(19) United States
(12) Patent Application Publication
(10) Pub. No.: US 2023/0304031 A1
(43) Pub. Date: Sep. 28, 2023

(54) VECTORS AND METHODS FOR IN VIVO TRANSDUCTION

(71) Applicant: Abintus Bio, Inc., San Diego, CA (US)

(72) Inventors: Douglas J. Jolly, Encinitas, CA (US); Derek G. Ostertag, San Diego, CA (US); Noriyuki Kasahara, Belvedere Tiburon, CA (US); Carlos Ibanez, San Diego, CA (US); Cornelia A. Bentley, San Diego, CA (US); Mohd Mushtaq Husain, Solana Beach, CA (US)

(21) Appl. No.: 17/924,080

(22) PCT Filed: May 11, 2021
(86) PCT No.: PCT/US2021/031878

§ 371 Date: Nov. 8, 2022

Related U.S. Application Data
(60) Provisional application No. 63/133,224, filed on Dec. 31, 2020.
(60) Provisional application No. 63/023,191, filed on May 11, 2020.

(51) Int. Cl. C12N 15/86; C07K 14/725; C07K 16/18; C12N 15/113; C12N 9/78; C12N 9/12; A61K 35/76; A61P 35/00; A61K 48/00

(52) U.S. Cl. CPC C12N 15/86; A61K 35/76; A61K 48/005; A61P 35/00; C07K 14/7051; C07K 16/18; C12N 9/1211; C12N 9/78; C12N 15/113

(57) ABSTRACT

The disclosure provides compositions and methods for inducing an immunity via cellular expression of an antigen receptor binding construct in vivo.

### CROSS REFERENCE TO RELATED APPLICATIONS

This application claims priority under 35 U.S.C. §119 from U.S. Provisional Application Serial No. 63/023,191, filed May 11, 2020 and U.S. Provisional Application Serial No. 63/133,224, filed Dec. 31, 2020, the disclosures of which are incorporated herein by reference.

### TECHNICAL FIELD

The disclosure provides compositions and methods for cellular therapies of cancer, infection, allergic, degenerative and immune disorders.

### INCORPORATION BY REFERENCE OF SEQUENCE LISTING

Accompanying this filing is a Sequence Listing entitled “Sequence-Listing_ST25.txt”, created on May 11, 2021 and having 239,881 bytes of data, machine formatted on IBM-PC, MS-Windows operating system. The sequence listing is hereby incorporated herein by reference in its entirety for all purposes.

### BACKGROUND

Adoptive immunotherapy has risen to the forefront of treatment approaches for cancer. T cells can be engineered to express the genes of chimeric antigen receptors (CARs) that recognize tumor associated antigens. CARs are engineered immune-receptors, which can redirect T cells to selectively kill tumor cells. The general premise for their use in cancer immunotherapy is to rapidly generate tumor-targeted T cells.

### SUMMARY

The ideal gene delivery system would be easy to produce, easy to administer, and non-toxic to normal cells; it would deliver the genetic information efficiently and specifically via the bloodstream to the targeted tissues and integrate the genetic material into the host cell so that the transgene would be stably expressed. The novel systems described here fit this prescription.

The disclosure provides a recombinant vector comprising (a) a polynucleotide encoding a chimeric antigen receptor (CAR); and (b) polynucleotide comprising at least one miRNA targeting sequence, wherein (a) and (b) are linked on the same polynucleotide. In one embodiment, the vector further comprising (c) a polynucleotide encoding a cytotoxic polypeptide that converts a prodrug to a cytotoxic drug. In another embodiment, (a) comprises a first polynucleotide domain encoding one or more antigen binding domain(s); an optional polynucleotide domain encoding a linker; and a second polynucleotide domain operably linked to the first polynucleotide domain, wherein the second polynucleotide domain encodes a transmembrane; and a third polynucleotide domain encoding an intracellular signaling domain. In a further embodiment, the first polynucleotide domain encodes an antibody fragment, single domain antibody, single chain variable fragment, single domain antibody, camelid VHH domain, a non-immunoglobulin antigen binding scaffold, a receptor or receptor fragment, or a bispecific antibody. In another embodiment, the optional polynucleotide encoding a linker encodes a Gly3 sequence. In still another embodiment, the transmembrane domain is from a member selected from the group consisting alpha, beta or zeta chain of a T-cell receptor, CD3γ, CD3ε, CD35, CD28, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRFl), CD160, CD19, IL2R beta, IL2R gamma, IL7R a, ITGA1, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In yet another embodiment, the third polynucleotide domain encodes an intracellular signaling domain selected from the group consisting of CD3 zeta, common FeR gamma (FCER1G), Fe gamma RIIa, FeR beta (Fe Epsilon R1b), CD3 gamma, CD3 delta, CD3 epsilon, CD79a, CD79b, DAPlo, and DAP12. In one embodiment, the cytotoxic polypeptide that converts a prodrug to a cytotoxic drug is selected from the group consisting of a polypeptide having cytosine deaminase activity, a polypeptide having thymidine kinase activity and a combination thereof. In any of the foregoing embodiments, the vector is an integrating vector. In a further embodiment, the vector is a retroviral vector. In still a further embodiment, the retroviral vector is a non-replicating gammaretroviral vector. In another embodiment, the at least one miRNA targeting sequence is bound by an miRNA selected from the group consisting of hsa-miR-223-3p, hsa-miR143-3p, hsa-mir182-5p, hsa-miR-10bp, hsa-miR141-3p, has-miR486-5p and any combination of the foregoing.

The disclosure also provides a recombinant retroviral particle comprising a gag polypeptide; a pol polypeptide; an env polypeptide; and a retroviral polynucleotide contained within a capsid of the retroviral vector, wherein the retroviral polynucleotide comprises from 5′ to 3′: (R-U5 domain)-(optional signal peptide coding sequence domain)-(Binding domain coding sequence domain)-(optional hinge/linker coding sequence domain)-(transmembrane (TM) coding sequence domain)-(miRNA target domain(s))-(U3-R domain). In one embodiment, the R-U5 domain has a sequence that is at least 80% identical to SEQ ID NO:25 from nucleotide 1 to about nucleotide 145. In another or further embodiment, the binding domain coding sequence is preceded by a signal sequence. In still another or further embodiment, the binding domain coding sequence is followed by an optional linker/spacer domain sequence. In still another or further embodiment, the retroviral polynucleotide further comprises a kill switch domain coding sequence. In a further embodiment, the kill switch coding domain comprises an IRES operably linked to a coding sequence for a polypeptide that converts a prodrug to a cytotoxic drug. In a further embodiment, the polypeptide has thymidine kinase (TKO) activity or cytosine deaminase (CD) activity. In another embodiment, the retroviral polynucleotide comprises at least one miRNA targeting sequence. In a further embodiment, the at least one miRNA targeting sequence comprises a plurality of miRNA targeting sequences. In still a further embodiment, the plurality of miRNA targeting sequences are the same. In yet another embodiment, at least two of the plurality of miRNA targeting sequences are different. In another embodiment, the U3-R domain comprises a sequence that is at least 80% identical to SEQ ID NO:25 from about nucleotide 5537 to about 6051.

The disclosure further provides a non-human animal carrying gene transduced stem cells produced by in vivo transduction using a vector of the disclosure.

### BRIEF DESCRIPTION OF THE FIGURES

FIG. 1 is an illustration of RNV engineered to deliver CAR or GFP transgenes with or without varying 3′ UTR microRNA target sequences. Examples include miR233 (causes transcript degradation in monocytes); or miRaBCT and miRaNKT (cause transcript degradation in B and NK cells, respectively). These target sequences are added in multiples of 1-4 and in varying combinations to direct transgene degradation in monocytes, B, and NK cells.

FIG. 2 is an illustration of RNV engineered to deliver CAR or GFP transgenes with or without varying 3′ UTR microRNA target sequences. In addition to FIG. 1, further examples include miR122a and miR199a which specifies transgene transcript degradation in hepatocytes. These target sequences are added in multiples of 1-4 and in varying combinations with miRNA target sequence describe in FIG. X1 to direct transgene

FIG. 3 provides an expected CD19 CAR expression profile in peripheral T-cells from mixed lymphocyte (PBMC) transduction. In vitro infected PBMC cells are stained with CD3, CD20, CD4, CD8, CD45 and CD19CAR-specific antibodies and are analyzed by flow cytometry. CD3, CD4 and CD8 positive cells show CD19CAR positive staining (APC) compared to RNV-GFP infected PBMCs.

FIGS. 4A-C provide schematic presentations of the three safety-modified MLV-based retroviral components amphotropic env (4070A-derived), gag/pol (MoMLV-derived), and vector (MoMLV-derived). (A) In the pKT-1 retroviral vector, extraneous sequences (denoted in black) upstream of the 5′ LTR, downstream of the 3′ LTR and between the unique ClaI site and the TAG env stop codon were deleted. In addition, two stop codons were introduced in the extended packaging signal Ψ+ to prevent production of Gag/pol proteins. The first TAA stop codon replaces the ATG start codon of gag/pol, with the second TGA stop codon introduced 12 nt after the first TAA stop. All these changes were incorporated in the safety-modified vector pBA-5b. A polylinker was subsequently inserted into pBA5b to arrive at the plasmid vector used in this study, pBA9Bb. (B) In the original gag/pol construct pSCV10 all 5′ and 3′ untranslated sequences were removed and sequence coding for the last 28 amino acids of the integrase gene in Pol truncated (pSCV10/5′,3′tr. or pCI-GPM). Additionally, degenerate code was incorporated in the first 420 nt of gag/pol to prevent overlap to the extended packaging signal of the vector (pCI-WGPM). (C) The original amphotropic envelope construct pCMVenv^{am}Dra was modified to minimize sequence overlap such that either the 3′ untranslated sequences (pCMVenv^{am}DraLBGH) or the 5′ and 3′ untranslated sequences (pCMV-β/env^{am}) were deleted.

FIG. 5 depict constructs comprising microRNA target sequences used to down regulate expression of GFP sequences in myeloid, B and NK cells by using multiple miR sequences as described above.

FIG. 6 provide flow charts outlining the process for generating a packaging cell line (PCL) and high-titer vector-producing line (VPCL) starting with a parent cell line. For clinical manufacturing, selected clones are eventually expanded, cryopreserved and testing under GMP to create qualified GMP Master and Working cell banks.

FIGS. 7A-E show the results of an experiment in the A20 B-cell lymphoma animal model where (B) tumor-bearing mice injected IV with the vector of (A) mCD19 (1D3)-IRES YCD(V), at a dose of 1E7 per day for four consecutive days, starting at day 3 after A20 implantation. Tumor burden was assessed at days 12, 18 and 25 after A20 B cell lymphoma implantation by imaging (C) and luminescent signal (D, radiance) in vehicle control and RNV-1D3-treated animals. Imaging of luminescent signal show a visual decrease in tumor burden (C). The quantitative luminescent signal is assessed for each mouse on from both ventral and dorsal perspective (top and bottom images). (E) Measurements of B cell (CD19 positive) counts for treatment and control mice showed a significant lowering of B cell counts in treatment mice by day 25.

FIG. 8 shows a Western blot could confirm expression of yCD2 in cells transduced with construct 8 at high (MOI 10) and low MOI (0.1) compared to construct 14 which does not have the yCD2 transgene.

FIG. 9 shows kill-curves for CD and thymidine kinase (TK) encoding vectors: for CD encoding vectors +/- 5-FC (flucytosine, D.Ostertag et al. Neuro-Oncology 2012), and for TK vectors +/ganciclovir (GCV). Test cells are plated and after 24-48 h drug is added at the various concentrations shown; viability was determined after 5-7 days and to generate a kill curve and IC₅₀ determination using the MTS assay (Abcam ab197010). Results show IC₅₀ 1 to 3 logs greater IC50′s for cells carrying vectors without the kill-switches genes compared to those without.

FIGS. 10A-B shows the effect of including a miRNA target sequence in an RNV vector. The target sequence for the miR223-3p was inserted into a GFP vector to give the sequence pBA-9B-GFPmiR223-3pB-4TX (construct 7) and used to make infectious vector. miR223-3p is a microRNAs which is produced at significant concentrations only in monocytic or myeloid cells. (A) Conceptual picture of the desired result. (B) shows the expression of the original GFP vector (left column), GFP miR 223-3p vector (center column)and GFP vector with an irrelevant miRNA target for miRaBC, in the U937 monocytic cell line, showing a 190-100foldredictio in GFP expression in the GFPmiR223 infected cell line, compared to the two other vectors. All three vectors produced equivalent amounts of GFP in HT1080 fibrosarcoma cells or other non-monocytic cells.

FIG. 11 provides a representation of an anti-BCMA-CAR RNV structure.

FIG. 12 shows a box-plot example of miRNA expression identification.

FIG. 13 shows miRNA expression de-targeting example. The figure shows the expression of the original GFP vector (left column), GFP miR 223-3p vector (center column) and GFP vector with an irrelevant miRNA target for miRaBC, in the U937 monocytic cell line, showing a 100 fold reduction in GFP expression in the GFPmiR223 infected cell line, compared to the two other vectors.

FIG. 14 shows the 4070A amphotropic envelope modified to contain anti-CD8 scFV sequences in both orientations inserted into the proline rich region of the env sequence. The use of Alternative Chimeric Viral Envelopes for Cell-Specific Targeting to CD34+ hematopoietic stem cells using MuLV and Lenti Pseudotyping. Pseudotyping of lentiviral vectors to target lymphocytes has been discussed in the literature (Frank et.al (2019) Surface-Engineered Lentiviral Vectors for Selective Gene Transfer into Subtypes of Lymphocytes, Mol. Ther. (Vol. 12). The majority of chimeric pseudotying has occurred taking advantage of the dual targeting and fusion functions of the Measles and Nipah viral systems. For viral cell receptor attachment, Measles virus encode Hemagglutinin-neuramidase (HN) for attachment to sialic acid receptor; Glycoprotein (G) and Hemagglutinin (H) for attachment to proteinaceous receptors.

FIGS. 15A-B shows the proviral integrated forms of (A) the pBA-9b vector with the mouse CD19CAR construct based on the antimouse CD19-1D3 scFV monoclonal antibody linked to hinge-TM and signaling domains or (B) the pBA-9b vector with the human CD19CAR construct based on the anti-human CD19-FMC63 scFV monoclonal antibody linked to hinge-TM and signaling domains.

FIG. 16 provides microRNA target sequences used to down regulate expression of CAR sequences in myeloid, B and NK cells.

FIG. 17 provides examples of a SIN vector design that drives expression using the constitutive CMV promoter with the EFla enhancer ensuring expression of the human anti-CD19 CAR sequence. The vector includes an insertional ribosome entry site (IRES) sequence for expression of a human codon optimized thymidine kinase (TKO)gene or a yeast cytosine deaminase gene (yCD2) as a vector kill switch when exposed to its corresponding prodrug, followed by a woodchuck hepatitis post-translational (WPRE) element for enhanced expression of the gene of interest.

FIGS. 18A-D shows retroviral constructs comprising CRISPR/CAS9 system(s) to (A) CCR5, (B) CCR2, (C) CCR5 and CCR2, and (D) CCR5 and CCR2 with CRISPR/CAS9 driven by internal promoter.

FIGS. 19A-D shows retroviral constructs (A-D) to treat HIV infection or multiple sclerosis by blocking activity of the HIV co-receptors CCR5 and CCR2.

FIGS. 20A-D shows retroviral constructs (A-D) to treat HIV infection or multiple sclerosis using siRNA of the HIV co-receptors CCR5 and CCR2.

FIG. 21 shows retroviral construct to treat HIV infection or multiple sclerosis using CRISPR/CAS systems to CCR5 and CCR2.

FIG. 22 shows time line used in the protocol for mobilization and transduction in vivo of hematopoietic stem/progenitor cells (HSPCs).

FIG. 23 shows RNV-GFP in vivo transduction for 2 hours in mobilized Balb/c mice. Splenocytes harvested and examined after 3 days in culture for GFP transduction of HSPC by FACS analysis and MethoCultassay. Photomicrographs taken after FACS analysis (upper panels show GFP+cells under UV light, lower panels show phase contrast).

### DETAILED DESCRIPTION

As used herein and in the appended claims, the singular forms “a,” “an,” and “the” include plural referents unless the context clearly dictates otherwise. Thus, for example, reference to “a subject” includes a plurality of such subjects and reference to “the vector” includes reference to one or more vectors and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless otherwise defined, all technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March’s Advanced Organic Chemistry Reactions, Mechanisms and Structure 7^{th} ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, NY 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor NY, 2013); Kohler and Milstein, Derivation of specific antibody-producing tissue culture and tumor lines by cell fusion, Eur. J. Immunol. 1976 Jul, 6(7):511-9; Queen and Selick, Humanized immunoglobulins, U.S. Pat. No. 5,585,089 (1996 Dec); and Riechmann et al., Reshaping human antibodies for therapy, Nature 1988 Mar 24, 332(6162):323-7All headings and subheading provided herein are solely for ease of reading and should not be construed to limit the invention. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and specific examples are illustrative only and not intended to be limiting.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1 %, 0.5%, or even 0.1 % of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The Retroviridae family of viruses may be used to create vectors that integrate into their host genome and provide long-term gene expression to the transduced cell and its descendants. In general, gammaretroviral vectors, lentiviral vectors and foamy viral vectors are usable and useful to transduce cells including mobilized stem cells. The disclosure focuses on gammaretroviral vectors, but one skilled in the art will quickly realize that the invention can use all types of integrating vectors including viral and non-viral vectors such as adenoviral-retroviral hybrids, piggy-bac and sleeping beauty transposons etc. The disclosure provides compositions and methods to transduce cells (including hematopoietic stem cells) in vivo by direct administration of the vector to achieve therapeutic effects in many typed of disease including genetic diseases, cancer, infectious disease and autoimmune disease. In some embodiments, cells can be mobilized prior to in vivo infection.

The vector constructs of the disclosure can be considered modular with domains (sometimes referred to as cassettes) described below. Generally, the vector comprises Long Terminal Repeats, a binding domain, a hinge or linker domain, a transmembrane domain, an intracellular domain, one or more miRNA target domains, an optional kill switch domain and an optional cell-activity-regulating domain. The binding domain, hinge/linker, transmembrane domain and intracellular domain generally comprise chimeric antigen receptors (CAR) including 1^{st} generation, 2^{nd} generation, 3^{rd} generation and related constructs.

The term “antibody,” as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be ‘humanized’, ‘chimeric’ or non-human.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, Fv fragments, scFv antibody fragments, disulfide-linked Fvs, a Fd fragment consisting of the VH and CHl domains, linear antibodies, single domain antibodies (sdAb) such as either vL or vH, camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No.: 6,703,199, which describes fibronectin polypeptide minibodies).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

“Anticancer agent” refers to agents that inhibit aberrant cellular division and growth, inhibit migration of neoplastic cells, inhibit invasiveness or prevent cancer growth and metastasis. The term includes chemotherapeutic agents, biological agent (e.g., siRNA, viral vectors such as engineered MLV, adenoviruses, herpes virus that deliver cytotoxic genes), antibodies and the like.

The term “anticancer effect” refers to a biological effect which can be manifested by various means including, but not limited to, a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anticancer effect” can also be manifested by the ability of a CAR in prevention of the occurrence of cancer in the first place.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. The disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

Non-limiting examples of antigens that can be targeted include: CD5; CD19; CD20; CD22; CD24; CD30; CD33, CD34; CD38, CD72; CD97; CD123; CD171; CS1 (also referred to as CD2 subset 1, CRACC, MPL, SLAMF7, CD319, and 19A24); C-type lectin-like molecule-1 (CLL-1 or CLECL1); epidermal growth factor receptor variant III (EGFRviii); ganglioside G2 (GD2); ganglioside GD3 (aNeu5Ac(2-8)aNeu5Ac(2-3)bDGalp(l-4 )bDGlcp(l-l)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD44v6; a glycosylated CD43 epitope; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; Platelet-derived growth factor receptor beta (PDGFR-beta); Stage-specific embryonic antigen-4 (SSEA-4); Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor); carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1- 4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member lA (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8); melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin Bl; v-myc avian myelocytomatosis viral oncogene neuroblastoma derived homolog (MYCN); Ras Homolog Family Member C (RhoC); Tyrosinase-related protein 2 (TRP-2); Cytochrome P450 lB 1 (CYPlB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TESl); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLLl); MPL; Biotin; c-MYC epitope Tag; LAMP1 TROP2; GFRalpha4; CDH17; CDH6; NYBR1; CDH19; CD200R; Slea (CA19.9; Sialyl Lewis Antigen); Fucosyl-GM1; PTK7; gpNMB; CDH1-CD324; DLL3; CD276/B7H3; IL11Ra; IL13Ra2; CD179b-IGLl1; TCRgamma-delta; NKG2D; CD32 (FCGR2A) ; Tn ag; Tim1-/HVCR1; CSF2RA (GM-CSFR-alpha); TGFbetaR2; Lews Ag; TCR-beta1 chain; TCR-beta2 chain; TCR-gamma chain; TCR-delta chain; FITC; Leutenizing hormone receptor (LHR); Follicle stimulating hormone receptor (FSHR) ; Gonadotropin Hormone receptor (CGHR or GR) ; CCR4; GD3; SLAMF6; SLAMF4; HIV1 envelope glycoprotein; HTLV1-Tax; CMV pp65; EBV-EBNA3c; KSHV K8.1; KSHV-gH; influenza A hemagglutinin (HA); GAD; PDL1; Guanylyl cyclase C (GCC); auto antibody to desmoglein 3 (Dsg3); auto antibody to desmoglein 1 (Dsg1); HLA; HLA-A; HLA-A2; HLA-B; HLA-C; HLA-DP; HLA-DM; HLA-DOA; HLA-DOB; HLA-DQ; HLA-DR; HLA-G; IgE; CD99; Ras G12V; Tissue Factor 1 (TF1); AFP; GPRC5D; Claudin18.2 (CLD18A2 or CLDN18A.2); P-glycoprotein; STEAP1; Liv1; Nectin-4; Cripto; gpA33; BST1/CD157; low conductance chloride channel; and the antigen recognized by TNT antibody.

An “antigen binding domain” refers to a polypeptide or peptide that due to its primary, secondary or tertiary sequence, post-translational modifications and/or charge binds to an antigen with a high degree of specificity. The antigen binding domain may be derived from different sources, for example, an antibody (full length heavy chain, Fab fragments, single chain Fv (scFv) fragments, divalent single chain antibodies or diabodies), a non-immunoglobulin binding protein, a ligand or a receptor. There are, however, numerous alternatives, such as linked cytokines (which leads to recognition of cells bearing the cytokine receptor), affibodies, ligand binding domains from naturally occurring receptors, soluble protein/peptide ligand for a receptor (for example on a tumor cell), peptides, and vaccines to prompt an immune response, which may each be used in various embodiments of the disclosure. In some embodiments, almost any molecule that binds a given cognate or antigen with high affinity can be used as an antigen binding domain, as will be appreciated by those of skill in the art. In some embodiments, the antigen binding domain comprises T cell receptors (TCRs) or portions thereof.

The term “anti-infection effect” refers to a biological effect that can be manifested by various means, including but not limited to, e.g., decrease in the titer of the infectious agent, a decrease in colony counts of the infectious agent, amelioration of various physiological symptoms associated with the infectious condition.

The term “antitumor effect” or “anti-cancer effect” refers to a biological effect which can be manifested by various means, including but not limited to, e.g., a decrease in tumor volume, a decrease in the number of tumor cells, a decrease in tumor cell proliferation, inhibition of metastasis, or a decrease in tumor cell survival.

As used herein “beneficial results” may include, but are not limited to, lessening or alleviating the severity of the disease condition, preventing the disease condition from worsening, curing the disease condition, preventing the disease condition from developing, lowering the chances of a patient or subject developing the disease condition and prolonging a patient’s or subject’s life or life expectancy. As non-limiting examples, “beneficial results” may be alleviation of one or more symptom(s), diminishment of extent of the deficit, stabilized (i.e., not worsening) state of cancer progression, delay or slowing of metastasis or invasiveness, and amelioration or palliation of symptoms associated with the cancer.

As used herein, the term “biological equivalent thereof” is intended to be synonymous with “equivalent thereof” when referring to a reference protein, antibody or fragment thereof, polypeptide or nucleic acid, intends those having minimal homology while still maintaining desired structure and/or functionality. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and exhibits substantially equivalent biological activity to the reference protein, polypeptide, antibody or fragment thereof or nucleic acid. Alternatively, when referring to polynucleotides, an equivalent thereof is a polynucleotide that hybridizes under stringent conditions to the reference polynucleotide or its complement and which has the same biological function (e.g., binds to a specific miRNA or encodes a protein or polypeptide having the same or similar biological effect to the polynucleotide to which it is being compared). Alternatively, when referring to polypeptides or proteins, an equivalent thereof is an expressed polypeptide or protein from a polynucleotide that hybridizes under stringent conditions to the polynucleotide or its complement that encodes the reference polypeptide or protein.

“Cancer” and “cancerous” refer to or describe the physiological condition in mammals that is typically characterized by unregulated cell growth. Examples of cancer include, but are not limited to, B-cell lymphomas (Hodgkin’s lymphomas and/or non-Hodgkin’s lymphomas), T cell lymphomas, myeloma, myelodysplastic syndrome, myeloproliferative disorders (e.g., polycythemia vera, myelofibrosis, essential thrombocythemia etc.), skin cancer, brain tumor, breast cancer, colon cancer, rectal cancer, esophageal cancer, anal cancer, cancer of unknown primary site, endocrine cancer, testicular cancer, lung cancer, hepatocellular cancer, gastric cancer, pancreatic cancer, cervical cancer, ovarian cancer, liver cancer, bladder cancer, cancer of the urinary tract, cancer of reproductive organs thyroid cancer, renal cancer, carcinoma, melanoma, head and neck cancer, brain cancer (e.g., glioblastoma multiforme), prostate cancer (including but not limited to androgen-dependent prostate cancer and androgen-independent prostate cancer), and leukemia. Other cancer and cell proliferative disorders will be readily recognized in the art. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. The term “cancer” is meant to include all types of cancerous growths or oncogenic processes, metastatic tissues or malignantly transformed cells, tissues, or organs, irrespective of histopathologic type or stage of invasiveness.

The term “cell-activity-regulating domain” refers to any one or more of PDL1, PDL2, CD80, CD86, crmA, p35, NEMO-K277A (or derivative thereof), K13-opt, IKK2-SS/EE, IKK1-SS/EE, 41BBL, CD40L, vFLIP-K13, MC159, and the like and combination thereof that is expressed in an immune cell (e.g., T cell, e.g., CAR-T cell etc.) to decrease, regulate or modify the activity of the immune cell. In some embodiments, an accessory module is co-expressed with an immune receptor such as a CAR to increase, decrease, regulate or modify the expression or activity of a CAR or a CAR-expressing cell. The accessory module can be co-expressed with a CAR using a single vector or using two or more different vectors.

“Chemotherapeutic agents” are compounds that are known to be of use in chemotherapy for cancer. Non-limiting examples of chemotherapeutic agents can include alkylating agents such as thiotepa and CYTOXAN® cyclosphosphamide; alkyl sulfonates such as busulfan, improsulfan and piposulfan; a camptothecin (including the synthetic analogue topotecan); bryostatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE; vinorelbine; novantrone; teniposide; edatrexate; daunomycin; aminopterin; xeloda; ibandronate; irinotecan (Camptosar, CPT-11); topoisomerase inhibitor RFS 2000; difluoromethylornithine (DMFO); retinoids such as retinoic acid; capecitabine; combretastatin; leucovorin (LV); oxaliplatin, lapatinib (Tykerb); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above or combinations thereof.

“Chimeric antigen receptors” (CARs) are artificial (non-naturally occurring) immune cell (e.g., T cell) receptors contemplated for use as a therapy for cancer, autoimmune diseases and infectious diseases, using a technique called adoptive cell transfer. CARs are also known as artificial T-cell receptors, chimeric T-cell receptors or chimeric immunoreceptors. CARs are constructed specifically to stimulate T cell activation and proliferation in response to a specific antigen to which the CAR binds. Generally, a CAR refers to a set of polypeptides, typically two in the simplest embodiments, which when expressed in an immune effector cell, provides the cell with specificity for a target antigen or cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some embodiments, the set of polypeptides are contiguous with each other. In one embodiment, the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In various embodiments, CARs are recombinant polypeptides comprising an antigen binding domain, a hinge region (HR), a transmembrane domain (TMD), an optional co-stimulatory domain (CSD) and an intracellular signaling domain (ISD). The optional costimulatory domain is generally absent in 1^{st} generation CAR constructs. Second (2^{nd}) generation CARs comprising antigen binding domains (e.g., vL and vH fragments, vHH, ligands and receptors etc.) typically incorporate a costimulatory domain (e.g., 41BB). Unless specified otherwise, as used herein, the term “CAR” or “CARs” also encompasses newer approaches to conferring antigen specificity onto cells, such as Antibody-TCR chimeric molecules or Ab-TCR (WO2017/070608A1 incorporated herein by reference), TCR receptor fusion proteins or TFP (WO2016/187349A1 incorporated herein by reference), Trifunctional T cell antigen coupler (Tri-TAC or TAC) (see, WO2015/117229A1, incorporated herein by reference). Typically, the term “CAR-T cell” is used, to refer to T-cells that have been engineered to express a chimeric antigen receptor. Thus, T lymphocytes bearing such CARs are generally referred to as CAR-T lymphocytes. CARs can be also expressed in cells other than T cells, such as hematopoietic stem cells, induced pluripotent stem cells (iPSC), NK cells and macrophage.

“Codon optimization” or “controlling for species codon bias” refers to the preferred codon usage of a particular host cell. As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. As part of a codon optimization, the coding sequences of a vector of the disclosure can be modified to limit ApoBec mediated mutations. In one embodiment, the vectors of the disclosure can be engineered to modify their stability and/or expression. For example, changes in expression can occur due to the frequency with which inactivating or attenuating mutations accumulate in the vector as it replicates in a cell. Investigation shows that one of the most frequent events is G to A mutations (corresponds to the C to T mutation) characteristic ApoBec mediated mutations in the negative strand of single stranded DNA from the first replicative step. This can cause changes in amino acid composition of vector-encoded proteins and a devastating change from TGG (Tryptophan) to stop codons (TAG or TGA). In one embodiment this inactivating change is avoided by substitution codons of other amino acids with similar chemical or structural properties such as phenylalanine or tyrosine at position of ApoBec modifications.

Such mutations can include modifications of one or more codons in the coding sequences of vector domains that change a tryptophan codon to a permissible codon that maintains the biological activity of the encoded protein. It is known in the art that the codon for tryptophan is UGG (TGG in DNA). Moreover, it is known in the art that the “stop codon” is UAA, UAG or UGA (TAA, TAG or TGA in DNA). A single point mutation in the tryptophan codon can cause an unnatural stop codon (e.g., UGG -> UAG or UGG -> UGA). It is also known that human APOBEC3GF (hA3G/F) inhibits retroviral replication through G -> A hypermutations (Neogi et al., J. Int. AIDS Soc., 16(1):18472, Feb. 25, 2013). Thus, the disclosure contemplates modifications to the coding sequences of vectors of the disclosure to reduce ApoBec hypermutations by modifying tryptophan codons to permissible non-tryptophan codons.

A “conservative substitution” or “conservative sequence modifications” refers to amino acid modifications that do not significantly affect or alter the binding characteristics or function of the encoded protein. For example, “conservative sequence modifications” refers to amino acid modifications that do not significantly affect or alter the binding characteristics or function of a CAR construct of the disclosure (e.g., a conservative change in the constant chain, antibody, antibody fragment, or non-immunoglobulin binding domains). Such conservative modifications include amino acid substitutions, additions and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the disclosure can be replaced with other amino acid residues from the same side chain family and the altered CAR can be tested using the binding and/or functional assays described herein.

“Co-stimulatory domain” as used herein refers to a biological agent that enhances the proliferation, survival and/or development of T cells. A co-stimulatory domain can comprises the costimulatory domain of any one or more of, for example, members of the TNFR superfamily, CD28, CD137 (4-1BB), CD134 (OX40), Dap10, CD27, CD2, CD5, ICAM-1, LFA-1(CD11a/CD18), Lck, TNFR-I, TNFR-II, Fas, CD30, CD40 or combinations thereof. Other co-stimulatory domains (e.g., from other proteins) will be apparent to those of skill in the art and may be used in connection with alternate embodiments of the disclosure.

Cytokine Release Syndrome (CRS) is a complication of cell therapies (e.g., CAR-T, bispecific T cell engaging antibodies etc.) that manifests itself with signs and symptoms such as fever, hypotension, shortness of breath, renal dysfunction, pulmonary dysfunction and/or capillary leak syndrome. CRS is usually due to excessive production of cytokines, such as IL6 and IL1.

“Derived from” as that term is used herein, indicates a relationship between a first and a second molecule. It generally refers to structural similarity between the first molecule and a second molecule and does not include a process or source limitation on a first molecule that is derived from a second molecule. For example, in the case of an antigen binding domain that is derived from an antibody molecule, the antigen binding domain retains sufficient antibody structure such that is has the required function, namely, the ability to bind to an antigen. It does not include any limitation to a particular process of producing the antibody.

“Domain” or “module” refers to a discrete section or part of a larger construct that can be replaced with a similar domain without affecting the function of other domains or module of the construct. For example, in a chimeric antigen receptor polypeptide or coding nucleic acid sequence can be described as having a binding domain, a transmembrane domain and an intracellular domain. Each “domain” of the CAR can be modified or changed without affecting the other domains of the CAR. For example, the binding domain can be one of a number of different binding domains as described herein. The binding domain can be a polypeptide sequence that binds to a CD19 antigen. This CD19 binding domain can be replaced with a binding domain that binds to CD20 without affecting of having to change the transmembrane domain. Similarly, a retroviral vector of the disclosure contained in a viral capsid comprises a polynucleotide sense RNA strand having a number of domains including (from 5′ to 3′): 5′Repeat(5′R)-U5-packaging sequence—CAR sequence-(optional kill switch comprising an IRES domain linked to, e.g., thymidine kinase coding sequence)-miRNA targeting sequence(s)-U3-3′Repeat(3′R). Each domain/module of the viral polynucleotide can be changed such that different CAR sequences can be provided, different kill switches (e.g., TKO or CD), different miRNA targeting sequences etc. The construct of the disclosure are modular in design. Each domain/module of the construct, whether a polynucleotide construct or an encoded polypeptide construct can comprise minor variations in sequence so long as the variations do not destroy the biological activity of the domain. Thus, for example, a transmembrane domain can have 80-100% identity to a specific transmembrane sequence.

“Genetically modified cells”, “redirected cells”, “genetically engineered cells” or “modified cells” as used herein refer to cells that express, for example, a CAR. In some embodiments, the genetically modified cells comprise vectors that encode a CAR.

“Hinge region” (HR) as used herein refers to the hydrophilic region which is between the antigen binding domain and the transmembrane domain of a CAR. The hinge region includes, but is not limited to, Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of hinge regions include, but are not limited to, CD8a hinge, and artificial spacers made of polypeptides which may be as small as, for example, Gly₃ or CH1 and CH3 domains of IgGs (such as human IgG4). In some embodiments, the hinge region is any one or more of (i) a hinge, CH2 and CH3 regions of IgG4, (ii) a hinge region of IgG4, (iii) a hinge and CH2 of IgG4, (iv) a hinge region of CD8a, (v) a hinge, CH2 and CH3 regions of IgG1, (vi) a hinge region of IgG1 or (vi) a hinge and CH2 region of IgG1. Other hinge regions will be apparent to those of skill in the art and may be used in connection with alternate embodiments of the disclosure.

“Immune cell” as used herein refers to the cells of the mammalian immune system including, but not limited to, antigen presenting cells, B-cells, basophils, cytotoxic T-cells, dendritic cells, eosinophils, granulocytes, helper T-cells, leukocytes, lymphocytes, macrophages, mast cells, memory cells, monocytes, natural killer cells, neutrophils, phagocytes, plasma cells and T-cells. “In vivo Immune cells” refer to immune cells present in the body of a subject that have not been isolated or removed from the subject.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

An “intracellular signaling domain,” (ISD) or “cytoplasmic domain” refers to an intracellular signaling portion of a molecule. The intracellular signaling domain generates a signal that promotes an immune effector function of the cell. Examples of immune effector function include cytolytic activity and helper activity, including the secretion of cytokines. Examples of domains that transduce the effector function signal include, but are not limited to, the z chain of the T-cell receptor complex or any of its homologs (e.g., h chain, FceR1g and b chains, MB1 (Iga) chain, B29 (Igb) chain, etc.), human CD3 zeta chain, CD3 polypeptides (D, d and e), syk family tyrosine kinases (Syk, ZAP 70, etc.), src family tyrosine kinases (Lck, Fyn, Lyn, etc.) and other molecules involved in T-cell transduction, such as CD2, CD5 and CD28. Other intracellular signaling domains will be apparent to those of skill in the art and may be used in connection with alternate embodiments of the disclosure.

In another embodiment, the intracellular signaling domain can comprise a primary intracellular signaling domain. Exemplary primary intracellular signaling domains include those derived from the molecules responsible for primary stimulation, or antigen dependent simulation. In another embodiment, the intracellular signaling domain can comprise a costimulatory intracellular domain. Exemplary costimulatory intracellular signaling domains include those derived from molecules responsible for costimulatory signals, or antigen independent stimulation. For example, a primary intracellular signaling domain can comprise a cytoplasmic sequence of CD3z or CD3z1xx (Feucht et al. Nt. Med 2019), and a costimulatory intracellular signaling domain can comprise cytoplasmic sequence from co-receptor or costimulatory molecule, such as CD28 or 41BB.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FeR gamma (FCER1G), Fe gamma RIIa, FeR beta (Fe Epsilon R1b), CD3 gamma, CD3 delta, CD3 epsilon, CD79a, CD79b, DAPlo, and DAP12.

The term “isolated” as used herein refers to molecules or biologics or cellular materials being substantially free from other materials. In one embodiment, the term “isolated” refers to nucleic acid, such as DNA or RNA; protein or polypeptide; cell or cellular organelle(s); or tissue, separated from other DNAs or RNAs, or proteins or polypeptides, or cells or cellular organelles, or tissues or organs, respectively, which are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both, cultured and engineered cells or tissues.

As used herein, the term “linker” (also “linker domain” or “linker region”) refers to an oligo or a peptide that joins together two or more domains or regions of a CAR polynucleotide or polypeptide, respectively. The linker can be anywhere from 1 to 500 amino acids in length or 3 to 1500 nucleotide in length. In some embodiments the “linker” is cleavable or non-cleavable. Unless specified otherwise, the term “linker” used herein means a non-cleavable linker. Said non-cleavable linkers may be composed of flexible amino acids residues which allow freedom of motion of adjacent protein domains relative to one another. Non-limiting examples of such residues include glycine and serine. In some embodiments, linkers include non-flexible amino acid residues. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof (e.g., GSG modified variants). In some embodiments, the linker sequences may comprise a motif that results in cleavage between the 2A glycine and the 2B proline. Other cleavable linkers that may be used herein are readily appreciated by those of skill in the art.

The term “flexible polypeptide linker” as used herein refers to a peptide linker that consists of amino acids such as glycine and/or serine residues used alone or in combination, to link polypeptide chains together (e.g., variable heavy and variable light chain regions together). In one embodiment, the flexible polypeptide linker is a Gly/Ser linker and comprises the amino acid sequence (Gly₃-Ser)ₙ, where n is a positive integer equal to or greater than 1 (e.g., 1, 2, 3, 4, 5, 6, 7, 8, 9, or 10 etc.).

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term. One of skill in the art will recognize that CAR constructs and related sequences are optimized for use in a particular mammalian species (e.g., the encoded protein/polypeptide is derived from the mammalian species being treated).

As used herein, “off-target transduced cells” refers to cells that are infected by a viral vector of the disclosure, but where expression of the vector’s genes are unwanted or undesirable. It will be recognized in the art that viral vectors can be “targeted” through incorporation of targeting proteins on the viral envelop. In addition, or alternatively, the expression of a viral gene can be controlled through the use of tissue specific promoters. In still other or further embodiments, the expression of the viral gene/construct can be controlled through the use of cellular machinery that exists to control innate gene expression control. In this instance RNAi target sequence can be used, whereby binding of innate miRNA to the target sequences can be used to control expression in an off-target cell type.

The term “operably linked” or “functionally linked” refers to functional linkage or association between a first component and a second component such that each component can be functional. For example, operably linked includes the association between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In the context of two polypeptides that are operably linked a first polypeptide functions in the manner it would independent of any linkage and the second polypeptide functions as it would absent a linkage between the two.

“Percent identity” in the context of two or more nucleic acids or polypeptide sequences, refers to two or more sequences that are related by percent sequence identity. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more typically over a region that is 100 to 500 or 1000 or more nucleotides (or 20, 50, 200 or more amino acids) in length.

For sequence comparison, generally one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art and are publicly available. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Bioi. 48:443, by the search for similarity method of Pearson and Lipman, (1988) Proc. Nat′l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., (2003) Current Protocols in Molecular Biology).

Two examples of algorithms that can be used for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Bioi. 215:403-410, respectively. Software for performing BLAST analyses is publicly available through the National Center for Biotechnology Information (NCBI).

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Bioi. 48:444-453) algorithm which has been incorporated into the GAP program in the GCG software package (available at www.gcg.com), using either a Blossom 62 matrix or a P AM250 matrix, and a gap weight of 16, 14, 12, 10, 8, 6, or 4 and a length weight of 1, 2, 3, 4, 5, or 6.

The term “RNAi target sequence”, or “miR target sequence” or “miR target cassette” as used herein, relates to a nucleic acid sequence specifically hybridizing to a dsRNA inducing RNA interference (interfering RNA). Thus, the RNAi target sequence is a sequence essentially complementary to at least one RNAi inducing molecule (interfering RNA). The RNAi target sequence is a miRNA target sequence or a siRNA target sequence. Typically the RNAi target sequence is a miRNA target sequence. As described more fully below the disclosure provides polynucleotide constructs containing a coding sequence for a CAR, one or more RNAi targeting sequences and an optional kill switch coding sequence.

The term “single chain variable region” or “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the vL and vH variable regions in either order, e.g., with respect to the N-terminal and C-terminal ends of the polypeptide, the scFv may comprise vL-linker-vH or may comprise vH-linker-vL. Alternatively, a scFv is also described as (vL+vH) or (vH+vL).

The term “signaling domain” refers to the functional region of a protein which transmits information within the cell to regulate cellular activity via defined signaling pathways by generating second messengers or functioning as effectors by responding to such messengers.

The term “subject” is intended to include living organisms in which an immune response can be elicited (e.g., any domesticated mammals or a human). The terms “subject” or “individual” or “animal” or “patient” are used interchangeably herein to refer to any subject, particularly a mammalian subject, for whom administration of a composition or pharmaceutical composition of the disclosure is desired. Mammalian subjects include humans, non-human primates, dogs, cats, guinea pigs, rabbits, rats, mice, horses, cattle, cows, and the like, with humans being preferred.

The terms “T-cell” and “T-lymphocyte” are interchangeable and used synonymously herein. Examples include but are not limited to naive T cells (“lymphocyte progenitors”), central memory T cells, effector memory T cells, stem memory T cells (T_{scm}), iPSC-derived T cells, synthetic T cells or combinations thereof.

The term “therapeutic effect” refers to a biological effect which can be manifested by various means, including but not limited to, e.g., decrease in tumor volume, a decrease in the number of cancer cells, a decrease in the number of metastases, an increase in life expectancy, decrease in cancer cell proliferation, decrease in cancer cell survival, decrease in the titer of the infectious agent, a decrease in colony counts of the infectious agent, amelioration of various physiological symptoms associated with a disease condition. A “therapeutic effect” can also be manifested by the ability of the peptides, polynucleotides, cells and antibodies in prevention of the occurrence of disease in the first place or in the prevention of relapse of the disease.

The term “therapeutically effective amount” as used herein refers to the amount of a pharmaceutical composition comprising vector or in vivo genetically engineered cells, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the a vector as described herein. Measured or measurable parameters include clinically detectable markers of disease, for example, elevated or depressed levels of a biological marker, as well as parameters related to a clinically accepted scale of symptoms or markers for cancer. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

“Transmembrane domain” (TMD) as used herein refers to the region of the CAR which crosses the plasma membrane. The transmembrane domain of the CAR of the disclosure is the transmembrane region of a transmembrane protein (for example Type I transmembrane proteins), an artificial hydrophobic sequence or a combination thereof. Other transmembrane domains will be apparent to those of skill in the art and may be used in connection with alternate embodiments of the disclosure. In some embodiments, the TMD is selected from the transmembrane domain of an alpha, beta or zeta chain of a T-cell receptor, CD3γ, CD3ε, CD3δ, CD28, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRFl), CD160, CD19, IL2R beta, IL2R gamma, IL7R a, ITGA1, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

“Vector”, “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc.

The term “viral vector” refers to a vector obtained or derived from a virus. Typically the virus is a retrovirus including, but not limited to, lentiviruses and gamma retroviruses. The viral vector of the disclosure may be a retroviral vector, such as a gamma-retroviral vector. The viral vector may be based on human immunodeficiency virus. The viral vector of the disclosure may be a lentiviral vector. The vector may be based on a non-primate lentivirus such as equine infectious anemia virus (EIAV). The viral vector of the disclosure comprises a mitogenic T-cell activating transmembrane protein and/or a cytokine-based T-cell activating transmembrane protein in the viral envelope. The mitogenic T-cell activating transmembrane protein and/or cytokine-based T-cell activating transmembrane protein is/are derived from the host cell membrane, as explained above.

As used herein “virus like particle” or “VLP” refers to a viral particle lacking a viral genome. In some cases the VLP lacks an env protein. As with complete viral particles they contain an outer viral envelope made of the host cell lipid-bi-layer (membrane), and hence contain host cell transmembrane proteins. A VLP can be used in the methods and compositions of the disclosure.

As described more fully below, the disclosure provides a recombinant viral vector comprising a plurality of copies of one or more miRNA target sequences inserted into the vector to control expression of coding sequence contained in the vector (e.g., CAR coding sequences) in off-target transduced cells. In certain embodiments, a recombinant viral vector may comprise miRNA target sequence(s) inserted into encapsidated viral polynucleotide. miRNAs expressed in off-target cells can bind to such miRNA target sequence(s) and suppress expression of the viral polynucleotide containing the miRNA target sequence, thereby limiting viral replication and/or expression of vector-containing coding sequences (e.g., CARs) in the off-target transduced cells. Such recombinant viral vectors can be referred to herein as “miR-attenuated”, “expression-restricted vectors” or “replication-restricted vectors” as they demonstrate reduced or attenuated replication and/or expression of vector-containing coding sequences in cells that express one or more miRNAs capable of binding to the incorporated miR target sequence(s) compared to cells that do not express, or have reduced expression of, the miR.

In certain embodiments, the one or more miRNA target sequence(s) is incorporated into the 3′ untranslated region (UTR) and/or 3′ UTR downstream of a CAR coding sequence. When transcribed, the mRNA transcripts of a CAR coding sequence comprises an miR-target sequence (TS) comprising one or more miRNA target sequences (e.g., a miRNA target sequence cassette). In some embodiments, the miR-TS cassettes described herein comprise at least one miRNA target sequence. In some embodiments, the miR-TS cassettes described herein comprise a plurality of miRNA target sequences. For example, in some embodiments, the miR-TS cassettes described herein comprise 2, 3, 4, 5, 6, 7, 8, 9, 10, 11, 12, 13, 14, 15, 16, 17, 18, 19, 20, or more miRNA target sequences. In such embodiments, wherein the miR-TS cassettes comprise two or more miRNA target sequences, the two or more target sequences can be the same of different.

In some embodiments, the miR-TS cassettes comprise a plurality miRNA target sequences, wherein each miRNA target sequence of the plurality is a target sequence for the same miRNA. For example, the miR-TS cassettes may comprise 2, 3, 4, 5, 6, 7, 8, 9, 10 or more copies of the same miR target sequence immediately contiguous or separated by a nucleotide spacer (e.g., 1-10 nucleotides). In some embodiments, the miR-TS cassettes comprise between 2 to 6 copies of the same miR target sequence. In some embodiments, the miR-TS cassettes comprise 3 copies of the same miR target sequence. In some embodiments, the miR-TS cassettes comprise 4 copies of the same miR target sequence. In some embodiments, the miR-TS cassettes comprise 5 copies of the same miR target sequence. In some embodiments, the miR-TS cassettes comprise 6 copies of the same miR target sequence. In some embodiments, the miR-TS cassettes comprise 7 copies of the same miR target sequence. In some embodiments, the miR-TS cassettes comprise 8 copies of the same miR target sequence. In some embodiments, the miR-TS cassettes comprise 9 copies of the same miR target sequence. In some embodiments, the miR-TS cassettes comprise 10 copies of the same miR target sequence.

In some embodiments, the miR-TS cassettes described herein comprise a plurality of miRNA target sequences, wherein the plurality comprises at least two different miRNA target sequences. In some embodiments, the miR-TS cassettes described herein comprise 2, 3, 4, 5, 6, 7, 8, 9, or 10 different miRNA target sequences. For example, in some embodiments, the miR-TS cassette may comprise one or more copies of a first miRNA target sequence and one or more copies of a second miRNA target sequence. In some embodiments, the miR-TS cassette comprises at least 2, 3, 4, 5, 6, 7, 8, 9, 10, or more copies of a first miR target sequence and at least 2, 3, 4, 5, 6, 7, 8, 9, 10, or more copies of a second miR target sequence. In some embodiments, the miR-TS cassette comprises 3 or 4 copies of a first miR target sequence and 3 or 4 copies of a second miR target sequence. In some embodiments, the plurality of miRNA target sequences comprises at least 3 different miRNA target sequences. For example, in some embodiments, the miR-TS cassette comprises one or more copies of a first miR target sequence, one or more copies of a second miR target sequence, and one or more copies of a third miR target sequence. In some embodiments, the miR-TS cassette comprises at least 2, 3, 4, 5, 6, 7, 8, 9, 10, or more copies of a first miR target sequence, at least 2, 3, 4, 5, 6, 7, 8, 9, 10, or more copies of a second miR target sequence, and at least 2, 3, 4, 5, 6, 7, 8, 9, 10, or more copies of a third miR target sequence. In some embodiments, the miR-TS cassette comprises 3 or 4 copies of a first miR target sequence, 3 or 4 copies of a second miR target sequence, and 3 or 4 copies of a third miR target sequence. In some embodiments, the plurality of miRNA target sequences comprises at least 4 different miRNA target sequences. For example, in some embodiments, the miR-TS cassette comprises one or more copies of a first miR target sequence, one or more copies of a second miR target sequence, one or more copies of a third miR target sequence, and one or more copies of a fourth miR target sequence. In some embodiments, the miR-TS cassette comprises at least 2, 3, 4, 5, 6, 7, 8, 9, 10, or more copies of a first miR target sequence, at least 2, 3, 4, 5, 6, 7, 8, 9, 10, or more copies of a second miR target sequence, at least 2, 3, 4, 5, 6, 7, 8, 9, 10, or more copies of a third miR target sequence, and at least 2, 3, 4, 5, 6, 7, 8, 9, 10, or more copies of a fourth miR target sequence. In some embodiments, the miR-TS cassette comprises 3 or 4 copies of a first miR target sequence, 3 or 4 copies of a second miR target sequence, 3 or 4 copies of a third miR target sequence, and 3 or 4 copies of a fourth miR target sequence.

In some embodiments, wherein the miR-TS cassettes comprise a plurality of miRNA target sequences, the plurality of miRNA target sequences may be arranged in tandem, without any intervening nucleic acid sequences. In some aspects, the plurality of miRNA target sequences may be separated by a linker sequence. In some embodiments, the linker sequence comprises 2, 3, 4, 5, 6, 7, 8, 9, 10, 15, 20, 25, or more nucleotides. In some embodiments, the linker sequence comprises about 4 to about 20 nucleotides. In further embodiments, the linker sequence comprises about 4 to about 16 nucleotides. As an illustrative embodiment, a miR-TS cassette may comprise 1, 2, 3, 4, 5, 6, 7, 8, 9, 10, or more of the following subunits: a first miRNA target sequence-linker-a second miRNA target sequence, wherein adjacent subunits are separated by an additional linker sequence. In some embodiments, the first and the second miRNA target sequence are targets of the same miRNA. In some embodiments, the first and the second miRNA target sequence are targets of different miRNAs.

In some embodiments, the miR target sequence is a target sequence for miR-1251-5p, miR-219a-5p, miR-219a-2-3p, miR-124-3p, miR-448, miR-138-2-3p, miR-490-5p, miR-129-1-3p, miR-1264, miR-3943, miR-490-3p, miR-383-5p, miR-133b, miR-129-2-3p, miR-128-2-5p, miR-133a-3p, miR-129-5p, miR-1-3p, miR-885-3p, miR-124-5p, miR-759, miR-7158-3p, miR-770-5p, miR-135a-5p, miR-885-5p, let-7g-5p, miR-100, miR-101, miR-106a, miR-124, miR-124a, miR-125a, miR-125a-5p, miR-125b, miR-127-3p, miR-128, miR-129, miR-136, miR-137, miR-139-5p, miR-142-3p, miR-143, miR-145, miR-146b-5p, miR-149, miR-152, miR-153, miR-195, miR-21, miR-212-3p, miR-219-5p, miR-222, miR-29b, miR-31, miR-3189-3p, miR-320, miR-320a, miR-326, miR-330, miR-331-3p, miR-340, miR-342, miR-34a, miR-376a, miR-449a, miR-483-5p, miR-503, miR-577, miR-663, miR-7, miR-7-5p, miR-873, let-7a, let-7f, miR-107, miR-122, miR-124-5p, miR-139, miR-146a, miR-146b, miR-15b, miR-16, miR-181a, miR-181a-1, miR-181a-2, miR-181b, miR-181b-1, miR-181b-2, miR-181c, miR-181d, miR-184, miR-185, miR-199a-3p, miR-200a, miR-200b, miR-203, miR-204, miR-205, miR-218, miR-23b, miR-26b, miR-27a, miR-29c, miR-328, miR-34c-3p, miR-34c-5p, miR-375, miR-383, miR-451, miR-452, miR-495, miR-584, miR-622, miR-656, miR-98, miR-124-3p, miR-181b-5p, miR-200b, and/or miR-3189-3p. In a further embodiment, the vector comprises a CAR coding sequence used to treat brain cancer.

In some embodiments, the miR target sequence is a target sequence for miR-10b-5p, miR-126-3p, miR-145-3p, miR-451a, miR-199b-5p, miR-5683, miR-3195, miR-3182, miR-1271-5p, miR-204-5p, miR-409-5p, miR-136-5p, miR-514a-5p, miR-559, miR-483-3p, miR-1-3p, miR-6080, miR-144-3p, miR-10b-3p, miR-6130, miR-6089, miR-203b-5p, miR-4266, miR-4327, miR-5694, miR-193b, let-7a, let-7a-1, let-7a-2, let-7a-3, let-7b, let-7c, let-7d, let-7e, let-7f-1, let-7f-2, let-7g, let-7i, miR-100, miR-107, miR-10a, miR-10b, miR-122, miR-124, miR-1258, miR-125a-5p, miR-125b, miR-126, miR-127, miR-129, miR-130a, miR-132, miR-133a, miR-143, miR-145, miR-146a, miR-146b, miR-147, miR-148a, miR-149, miR-152, miR-153, miR-15a, miR-16, miR-17-5p, miR-181a, miR-1826, miR-183, miR-185, miR-191, miR-193a-3p, miR-195, miR-199b-5p, miR-19a-3p, miR-200a, miR-200b, miR-200c, miR-205, miR-206, miR-211, miR-216b, miR-218, miR-22, miR-26a, miR-26b, miR-300, miR-30a, miR-31, miR-335, miR-339-5p, miR-33b, miR-34a, miR-34b, miR-34c, miR-374a, miR-379, miR-381, miR-383, miR-425, miR-429, miR-450b-3p, miR-494, miR-495, miR-497, miR-502-5p, miR-517a, miR-574-3p, miR-638, miR-7, miR-720, miR-873, miR-874, miR-92a, miR-98, miR-99a, mmu-miR-290-3p, and/or mmu-miR-290-5p. In a further embodiment, the vector comprises a CAR coding sequence used to treat breast cancer.

In some embodiments, the miR target sequence is a target sequence for miR-143, miR-145, miR-17-5p, miR-203, miR-214, miR-218, miR-335, miR-342-3p, miR-372, miR-424, miR-491-5p, miR-497, miR-7, miR-99a, miR-99b, miR-100, miR-101, miR-15a, miR-16, miR-34a, miR-886-5p, miR-106a, miR-124, miR-148a, miR-29a, and/or miR-375. In a further embodiment, the vector comprises a CAR coding sequence used to treat cervical cancer.

In some embodiments, the miR target sequence is a target sequence for miR-133a-5p, miR-490-5p, miR-124-3p, miR-137, miR-655-3p, miR-376c-3p, miR-369-5p, miR-490-3p, miR-432-5p, miR-487b-3p, miR-342-3p, miR-223-3p, miR-136-3p, miR-136-3p, miR-143-5p, miR-1-3p, miR-214-3p, miR-143-3p, miR-199a-3p, miR-199b-3p, miR-451a, miR-127-3p, miR-133a-3p, miR-145-5p, miR-145-3p, miR-199a-5p, let-7a-1, let-7a-2, let-7a-3, let-7b, let-7c, let-7d, let-7e, let-7f-1, let-7f-2, let-7g, let-7i, miR-100, miR-101, miR-126, miR-142-3p, miR-143, miR-145, miR-192, miR-200c, miR-21, miR-214, miR-215, miR-22, miR-25, miR-302a, miR-320, miR-320a, miR-34a, miR-34c, miR-365, miR-373, miR-424, miR-429, miR-455, miR-484, miR-502, miR-503, miR-93, miR-98, miR-186, miR-30a-5p, miR-627, let-7a, miR-1, miR-124, miR-125a, miR-129, miR-1295b-3p, miR-1307, miR-130b, miR-132, miR-133a, miR-133b, miR-137, miR-138, miR-139, miR-139-5p, miR-140-5p, miR-148a, miR-148b, miR-149, miR-150-5p, miR-154, miR-15a, miR-15b, miR-16, miR-18a, miR-191, miR-193a-5p, miR-194, miR-195, miR-196a, miR-198, miR-199a-5p, miR-203, miR-204-5p, miR-206, miR-212, miR-218, miR-224, miR-24-3p, miR-26b, miR-27a, miR-28-3p, miR-28-5p, miR-29b, miR-30a-3p, miR-30b, miR-328, miR-338-3p, miR-342, miR-345, miR-34a-5p, miR-361-5p, miR-375, miR-378, miR-378a-3p, miR-378a-5p, miR-409-3p, miR-422a, miR-4487, miR-483, miR-497, miR-498, miR-518a-3p, miR-551a, miR-574-5p, miR-625, miR-638, miR-7, miR-96-5p, miR-202-3p, miR-30a, and/or miR-451. In a further embodiment, the vector comprises a CAR coding sequence used to treat colon or colorectal cancer.

In some embodiments, the miR target sequence is a target sequence for miR-101, miR-130a, miR-130b, miR-134, miR-143, miR-145, miR-152, miR-205, miR-223, miR-301a, miR-301b, miR-30c, miR-34a, miR-34c, miR-424, miR-449a, miR-543, and/or miR-34b. In a further embodiment, the vector comprises a CAR coding sequence used to treat endometrial cancer.

In some embodiments, the miR target sequence is a target sequence for miR-125b, miR-138, miR-15a, miR-15b, miR-16, miR-16-1, miR-16-1-3p, miR-16-2, miR-181a, miR-181b, miR-195, miR-223, miR-29b, miR-34b, miR-34c, miR-424, miR-10a, miR-146a, miR-150, miR-151, miR-155, miR-2278, miR-26a, miR-30e, miR-31, miR-326, miR-564, miR-27a, let-7b, miR-124a, miR-142-3p, let-7c, miR-17, miR-20a, miR-29a, miR-30c, miR-720, miR-107, miR-342, miR-34a, miR-202, miR-142-5p, miR-29c, miR-145, miR-193b, miR-199a, miR-214, miR-22, miR-137, and/or miR-197. In a further embodiment, the vector comprises a CAR coding sequence used to treat hematologic cancer.

In some embodiments, the miR target sequence is a target sequence for miR-1, miR-145, miR-1826, miR-199a, miR-199a-3p, miR-203, miR-205, miR-497, miR-508-3p, miR-509-3p, let-7a, let-7d, miR-106a*, miR-126, miR-1285, miR-129-3p, miR-1291, miR-133a, miR-135a, miR-138, miR-141, miR-143, miR-182-5p, miR-200a, miR-218, miR-28-5p, miR-30a, miR-30c, miR-30d, miR-34a, miR-378, miR-429, miR-509-5p, miR-646, miR-133b, let-7b, let-7c, miR-200c, miR-204, miR-335, miR-377, and/or miR-506. In a further embodiment, the vector comprises a CAR coding sequence used to treat kidney cancer.

In some embodiments, the miR target sequence is a target sequence for let-7a-1, let-7a-2, let-7a-3, let-7b, let-7c, let-7d, let-7e, let-7f, let-7f-1, let-7f-2, let-7g, let-7i, miR-1, miR-100, miR-101, miR-105, miR-122, miR-122a, miR-1236, miR-124, miR-125b, miR-126, miR-127, miR-1271, miR-128-3p, miR-129-5p, miR-130a, miR-130b, miR-133a, miR-134, miR-137, miR-138, miR-139, miR-139-5p, miR-140-5p, miR-141, miR-142-3p, miR-143, miR-144, miR-145, miR-146a, miR-148a, miR-148b, miR-150-5p, miR-15b, miR-16, miR-181a-5p, miR-185, miR-188-5p, miR-193b, miR-195, miR-195-5p, miR-197, miR-198, miR-199a, miR-199a-5p, miR-199b, miR-199b-5p, miR-200a, miR-200b, miR-200c, miR-202, miR-203, miR-204-3p, miR-205, miR-206, miR-20a, miR-21, miR-21-3p, miR-211, miR-212, miR-214, miR-217, miR-218, miR-219-5p, miR-22, miR-223, miR-26a, miR-26b, miR-29a, miR-29b-1, miR-29b-2, miR-29c, miR-302b, miR-302c, miR-30a, miR-30a-3p, miR-335, miR-338-3p, miR-33a, miR-34a, miR-34b, miR-365, miR-370, miR-372, miR-375, miR-376a, miR-377, miR-422a, miR-424, miR-424-5p, miR-433, miR-4458, miR-448, miR-450a, miR-451, miR-485-5p, miR-486-5p, miR-497, miR-503, miR-506, miR-519d, miR-520a, miR-520b, miR-520c-3p, miR-582-5p, miR-590-5p, miR-610, miR-612, miR-625, miR-637, miR-675, miR-7, miR-877, miR-940, miR-941, miR-98, miR-99a, miR-132, and/or miR-31. In a further embodiment, the vector comprises a CAR coding sequence used to treat liver cancer. In further embodiments, the liver cancer is hepatocellular carcinoma.

In some embodiments, the miR target sequence is a target sequence for miR-143-3p, miR-126-3p, miR-126-5p, miR-1266-3p, miR-6130, miR-6080, miR-511-5p, miR-143-5p, miR-223-5p, miR-199b-5p, miR-199a-3p, miR-199b-3p, miR-451a, miR-142-5p, miR-144, miR-150-5p, miR-142-3p, miR-214-3p, miR-214-5p, miR-199a-5p, miR-145-3p, miR-145-5p, miR-1297, miR-141, miR-145, miR-16, miR-200a, miR-200b, miR-200c, miR-29b, miR-381, miR-409-3p, miR-429, miR-451, miR-511, miR-99a, let-7a-1, let-7a-2, let-7a-3, let-7b, let-7c, let-7d, let-7e, let-7f-1, let-7f-2, let-7g, let-7i, miR-1, miR-101, miR-133b, miR-138, miR-142-5p, miR-144, miR-1469, miR-146a, miR-153, miR-15a, miR-15b, miR-16-1, miR-16-2, miR-182, miR-192, miR-193a-3p, miR-194, miR-195, miR-198, miR-203, miR-217, miR-218, miR-22, miR-223, miR-26a, miR-26b, miR-29c, miR-33a, miR-34a, miR-34b, miR-34c, miR-365, miR-449a, miR-449b, miR-486-5p, miR-545, miR-610, miR-614, miR-630, miR-660, miR-7515, miR-9500, miR-98, miR-99b, miR-133a, let-7a, miR-100, miR-106a, miR-107, miR-124, miR-125a-3p, miR-125a-5p, miR-126, miR-126*, miR-129, miR-137, miR-140, miR-143, miR-146b, miR-148a, miR-148b, miR-149, miR-152, miR-154, miR-155, miR-17-5p, miR-181a-1, miR-181a-2, miR-181b, miR-181b-1, miR-181b-2, miR-181c, miR-181d, miR-184, miR-186, miR-193b, miR-199a, miR-204, miR-212, miR-221, miR-224, miR-27a, miR-27b, miR-29a, miR-30a, miR-30b, miR-30c, miR-30d, miR-30d-5p, miR-30e-5p, miR-32, miR-335, miR-338-3p, miR-340, miR-342-3p, miR-361-3p, miR-373, miR-375, miR-4500, miR-4782-3p, miR-497, miR-503, miR-512-3p, miR-520a-3p, miR-526b, miR-625*, and/or miR-96. In a further embodiment, the vector comprises a CAR coding sequence used to treat lung cancer.

In some embodiments, the miR target sequence is a target sequence for let-7b, miR-101, miR-125b, miR-1280, miR-143, miR-146a, miR-146b, miR-155, miR-17, miR-184, miR-185, miR-18b, miR-193b, miR-200c, miR-203, miR-204, miR-205, miR-206, miR-20a, miR-211, miR-218, miR-26a, miR-31, miR-33a, miR-34a, miR-34c, miR-376a, miR-376c, miR-573, miR-7-5p, miR-9, and/or miR-98. In a further embodiment, the vector comprises a CAR coding sequence used to treat melanoma.

In some embodiments, the miR target sequence is a target sequence for let-7d, miR-218, miR-34a, miR-375, miR-494, miR-100, miR-124, miR-1250, miR-125b, miR-126, miR-1271, miR-136, miR-138, miR-145, miR-147, miR-148a, miR-181a, miR-206, miR-220a, miR-26a, miR-26b, miR-29a, miR-32, miR-323-5p, miR-329, miR-338, miR-370, miR-410, miR-429, miR-433, miR-499a-5p, miR-503, miR-506, miR-632, miR-646, miR-668, miR-877, and/or miR-9. In a further embodiment, the vector comprises a CAR coding sequence used to treat oral cancer.

In some embodiments, the miR target sequence is a target sequence for let-7i, miR-100, miR-124, miR-125b, miR-129-5p, miR-130b, miR-133a, miR-137, miR-138, miR-141, miR-145, miR-148a, miR-152, miR-153, miR-155, miR-199a, miR-200a, miR-200b, miR-200c, miR-212, miR-335, miR-34a, miR-34b, miR-34c, miR-409-3p, miR-411, miR-429, miR-432, miR-449a, miR-494, miR-497, miR-498, miR-519d, miR-655, miR-9, miR-98, miR-101, miR-532-5p, miR-124a, miR-192, miR-193a, and/or miR-7. In a further embodiment, the vector comprises a CAR coding sequence used to treat ovarian cancer.

In some embodiments, the miR target sequence is a target sequence for miR-216a-5p, miR-802, miR-217, miR-145-3p, miR-143-3p, miR-451a, miR-375, miR-214-3p, miR-216b-3p, miR-432-5p, miR-216a-3p, miR-199b-5p, miR-199a-5p, miR-136-3p, miR-216b-5p, miR-136-5p, miR-145-5p, miR-127-3p, miR-199a-3p, miR-199b-3p, miR-559, miR-129-2-3p, miR-4507, miR-1-3p, miR-148a-3p, miR-101, miR-1181, miR-124, miR-1247, miR-133a, miR-141, miR-145, miR-146a, miR-148a, miR-148b, miR-150*, miR-150-5p, miR-152, miR-15a, miR-198, miR-203, miR-214, miR-216a, miR-29c, miR-335, miR-34a, miR-34b, miR-34c, miR-373, miR-375, miR-410, miR-497, miR-615-5p, miR-630, miR-96, miR-132, let-7a, let-7a-1, let-7a-2, let-7a-3, let-7b, let-7c, let-7d, let-7e, let-7f-1, let-7f-2, let-7g, let-7i, miR-126, miR-135a, miR-143, miR-144, miR-150, miR-16, miR-200a, miR-200b, miR-200c, miR-217, miR-218, miR-337, miR-494, and/or miR-98 inserted into the 5′ UTR or 3′ UTR of one or more viral genes required for viral replication. In a further embodiment, the vector comprises a CAR coding sequence used to treat pancreatic cancer.

In some embodiments, the miR target sequence is a target sequence for let-7a-3p, let-7c, miR-100, miR-101, miR-105, miR-124, miR-128, miR-1296, miR-130b, miR-133a-1, miR-133a-2, miR-133b, miR-135a, miR-143, miR-145, miR-146a, miR-154, miR-15a, miR-187, miR-188-5p, miR-199b, miR-200b, miR-203, miR-205, miR-212, miR-218, miR-221, miR-224, miR-23a, miR-23b, miR-25, miR-26a, miR-26b, miR-29b, miR-302a, miR-30a, miR-30b, miR-30c-1, miR-30c-2, miR-30d, miR-30e, miR-31, miR-330, miR-331-3p, miR-34a, miR-34b, miR-34c, miR-374b, miR-449a, miR-4723-5p, miR-497, miR-628-5p, miR-642a-5p, miR-765, and/or miR-940. In a further embodiment, the vector comprises a CAR coding sequence used to treat prostate cancer.

In some embodiments, the miR target sequence is a target sequence for miR-101, miR-183, miR-204, miR-34a, miR-365b-3p, miR-486-3p, and/or miR-532-5p. In a further embodiment, the vector comprises a CAR coding sequence used to treat retinoblastoma.

In some embodiments, the miR target sequence is a target sequence for miR-143-3p, miR-133b, miR-1264, miR-448, miR-1298-5p, miR-490-5p, miR-138-2-3p, miR-144-3p, miR-144-5p, miR-150-5p, miR-129-1-3p, miR-559, miR-1-3-p, miR-143-5p, miR-223-3p, miR-3943, miR-338-3p, miR-124-3p, miR-219a-5p, miR-219a-2-3p, miR-451a, miR-142-5p, miR-133a-3p, miR-145-5p, and/or miR-145-3p. In a further embodiment, the vector comprises a CAR coding sequence used to treat glioblastoma.

In some embodiments, the miR target sequence is a target sequence for miR-143-3p, miR-223-3p, miR-6080, miR-208b-3p, miR-206, miR-133a-5p, miR-133b, miR-199a-5p, miR-199b-5p, miR-145-3p, miR-145-5p, miR-150-5p, miR-142-3p, miR-144-3p, miR-144-5p, miR-338-3p, miR-214-3p, miR-559, miR-133a-3p, miR-1-3p, miR-126-3p, miR-142-5p, miR-451a, miR-199a-3p, and/or miR-199b-3p. In a further embodiment, the vector comprises a CAR coding sequence used to treat head and neck cancer.

Depending upon the designed CAR and the binding domain engineered into/onto the CAR, the methods and compositions can be used to treat a number of disease and disorders. For example, binding domains targeting any number of the “Targets” listing in Table 1 can be used to treat diseases associated with the target:

TABLE 1

| | |
|---|---|
| TARGET | EXEMPLARY DISEASE TARGETED BY CARs (i.e., conventional CARs and next generation CARs. E.g., Ab-TCR, and TFP) |
| CD19 | ALL, CLL, lymphoma, lymphoid blast crisis of CML, multiple myeloma, immune disorders |
| ALK | Non Small Cell Lung Cancer (NSCLC), ALCL (anaplastic large cell lymphoma), IMT (inflammatory myofibroblastic tumor), or neuroblastoma |
| CD45 | Blood cancers |
| BCMA | Myeloma, PEL, plasma cell leukemia, Waldenstrom’s macroglobinemia |
| CD5 | Blood cancer, T cell leukemia, T cell lymphoma |
| CD20 | Blood cancers, Leukemia, ALL, CLL, lymphoma, immune disorders |
| CD22 | Blood cancers, Leukemia, ALL, CLL, lymphoma, lymphoid blast crisis of CML, immune disorders |
| CD23 | Blood cancers, Leukemia, ALL, CLL, lymphoma, autoimmune disorders |
| CD30 | Hodgkins’s lymphoma, Cutaneous T cell lymphoma |
| CD32 | Solid tumors |
| CD33 | Blood cancers, AML, MDS |
| CD34 | Blood cancers, AML, MDS |
| CD44v6 | Blood cancers, AML, MDS |
| CD70 | Blood cancers, lymphoma, myeloma, Waldenstrom’s macroglobulinemia, Kidney cancer |
| CD79b | Blood cancers, ALL, Lymphoma |
| CD123 | Blood cancers, AML, MDS |
| CD138 | Blood cancers, Myeloma, PEL, plasma cell leukemia, waldenstrom’s macroglobulinemia |
| CD179b | Blood cancers, ALL, Lymphoma |
| CD276/B7-H3 | Ewing’s sarcoma, neuroblastoma, rhabdomyosarcoma, ovarian, colorectal and lung cancers |
| CD324 | Solid tumors, esophageal, prostate, colorectal, breast, lung cancers |
| CDH6 | Solid tumors, renal, ovarian, thyroid cancers |
| CDH17 | Adenocarciniomas, gastrointestinal, lung, ovarian, endometrial cancers |
| CDH19 | Solid tumor, Melanoma |
| EGFR | Colon cancer, lung cancer |
| CLEC5A | Blood cancers, Leukemia, AML |
| GR/LHR | Prostate cancer, ovarian cancer or breast cancer |
| CLL1 | Blood cancer, Leukemia |
| CMVpp65 | CMV infection, CMV colitis, CMV pneumonitis |
| CS1 | Blood cancers, myeloma, PEL, plasma cell leukemia |
| CSF2RA | AML, CML, MDS |
| CD123 | Blood cancers, AML, MDS |
| DLL3 | Melanoma, lung cancer or ovarian cancer |
| EBNA3c/MHC I | Epstein Barr virus infection and related diseases including cancers |
| EBV-gp350 | Epstein Barr virus infection and related diseases |
| EGFR | Solid tumors, Colon cancer, lung cancer |
| EGFRvIII | Solid tumors, glioblastoma |
| EpCam1 | Gastrointestinal cancer |
| FLT3 | Blood cancers, AML, MDS, ALL |
| Folate Receptor alpha | Ovarian cancer, NSCLC, endometrial cancer, renal cancer, or other solid tumors |
| FSHR | Prostate cancer, ovarian cancer or breast cancer |
| GD2 | Neuroblastoma |
| GD3 | Melanoma |
| GFRa4 | Cancer, thyroid medullary cancer |
| Fucosyl-GM1 | Small cell lung cancer |
| GPRC5D | Myeloma, PEL, plasma cell leukemia, waldenstrom’s macroglobulinemia |
| gp100 | Melanoma |
| GPC3 | Solid tumors, Lung cancer |
| gpNMB | Melanoma, brain tumors, gastric cancers |
| GRP78 | Myeloma |
| Her2 | Solid tumors, breast cancer, stomach cancer |
| Her3 | Colorectal, breast cancer |
| HMW-MAA | Melanoma |
| HTLV1-TAX/MHC I | HTLV1 infection associated diseases, Adult T cell leukemia-lymphoma |
| IL11Ra | Blood cancers, AML, ALL, CML, MDS, sarcomas |
| IL6Ra | Solid tumors, Liver cancer |
| IL13 Ra2 | Glioblastomas |
| KSHV-K8.1 | Kaposi’s sarcoma, PEL, Multicentric Castleman’s disease |
| LAMP1 | Blood cancers, AML, ALL, MDS, CLL, CML |
| LewisY | Cancers |
| L1CAM | Solid tumors, ovarian, breast, endometrial cancers, melanoma |
| LHR | Prostate cancer, ovarian cancer or breast cancer |
| Lym1 | Blood cancer, Leukemia, Lymphoma |
| Lym2 | Blood cancer, Leukemia, Lymphoma |
| CD79b | Blood cancers, lymphoma |
| MART1/MHC I | Melanoma |
| Mesothelin | Mesothelioma, ovarian cancer, pancreatic cancer |
| Muc1/MHC I | Breast cancer, gastric cancer, colorectal cancer, lung cancer, solid tumors |
| Muc16 | Ovarian cancer |
| NKG2D | Leukemia, lymphoma or myeloma |
| NYBR1 | Breast cancer |
| PSCA | Prostate cancer |
| PR1/MHC I | Blood cancer, Leukemia |
| Prolactin Receptor | Breast cancer, chromophobe renal cell cancer |
| PSMA | Prostate cancer |
| PTK7 | Melanoma, lung cancer or ovarian cancer |
| ROR1 | Blood cancer, B cell malignancy, lymphoma, CLL |
| SLea | Pancreatic cancer, colon cancer |
| SSEA4 | Pancreatic cancer |
| Tyrosinase/MHC I | Melanoma |
| TCRB1 | T cell leukemias and lymphomas, autoimmune disorders |
| TCRB2 | T cell leukemias and lymphomas, autoimmune disorders |
| TCRgd | T cell leukemias and lymphomas, autoimmune disorders |
| hTERT | Solid tumors, blood cancers |
| TGFBR2 | Solid tumors, keloid |
| TIM1/HAVCR1 | Kidney cancer, liver cancer |
| TROP2 | Solid tumors, Breast cancer,prostate cancer |
| TSHR | Thyroid cancer, T cell leukemia, T cell Lymphoma |
| TSLPR | Blood cancers, Leukemias, AML, MDS |
| Tyrosinase/MHC I | Melanoma |
| VEGFR3 | Solid tumors |
| WT1/MHC I | Blood cancers, AML |
| Folate Receptorβ | AML, Myeloma |
| B7H4 | Breast cancer or ovarian cancer |
| CD23 | Blood cancers, Leukemias, CLL |
| GCC | Gastrointestinal cancer |
| CD200R | Blood cancers, AML, MDS |
| AFP/MHC I | Solid tumors, Liver cancer |
| CD99 | Liver cancer |
| GPRC5D | Myeloma, waldenstrom’s macroglobinemia |
| HPV16-E7/MHC I | HPV16 associated cancers, cervical cancer, head and neck cancers |
| Tissue Factor 1 | Solid tumors |
| Tn-Muc1 | Solid tumors and blood cancers |
| Igk-Light Chain | Myeloma, plasma cell leukemia |
| Ras G12V/ MHC I | Solid tumors and blood cancers |
| CLD18A2 (Claudin 18.2) | Gastric, pancreatic, esophageal, ovarian, or lung cancer |
| CD43 | Blood cancers, AML |
| NYESO-1/MHCI | Myeloma |
| MPL/TPO-R | Blood cancer, AML, MDS, CML, ALL, Myeloproliferative disorders, Polycythemia vera, Myelofibrosis, Essential Polycythemia |
| P-glycoprotein (MDR1) | Renal cancer, liver cancer, Myeloma |
| CD179a | Blood cancers, Acute Leukemia, CLL, ALL, Lymphoma |
| STEAP1 | Gastric or prostate cancer, or lymphoma |
| Liv1 (SLC39A6) | Breast or prostate cancer |
| Nectin4 (PVRL4) | Bladder, renal, cervical, lung, head and neck or breast cancer |
| Cripto (TDGF1) | Colorectal or endometrial or ovarian cancer |
| gpA33 | Colorectal or endometrial or ovarian cancer |
| FLT3 | Blood cancers, AML, ALL, MDS |
| BST1/CD157 | Blood cancers, AML, MDS |
| IL1RAP | Liver, colorectal, cervical, lung or ovarian cancer |
| Chloride channel | Glioma |
| IgE | Allergy |
| HLA-A2 | Graft vs host disease, tissue rejection |
| Amyloid | Amyloidoses, alzheimer’s disease |
| HIV1-env | HIVI/AIDS and related conditions |
| HIV1-gag | HIV1/AIDS and related conditions |
| Influenza A HA | Influenza A infection |

The disclosure provides methods to provide adoptive cell immunity to a subject in need thereof comprising administering a vector of the disclosure encoding a chimeric antigen receptor (CAR) to the subject such that the CAR is selectively expressed in a desired immune cell type or immune cell stem cell (e.g., hematopoietic stem cell). The method includes administering a viral construct comprising a viral capsid and envelope containing a polynucleotide derived from a viral genome. In one embodiment, the polynucleotide comprises RNA. In another embodiment, the polynucleotide is derived from a gamma retrovirus. In still another embodiment, the polynucleotide comprises long terminal repeats at the 5′ and 3′ end. In yet another embodiment, the polynucleotide comprises a coding sequence for a CAR. In yet another embodiment, the polynucleotide comprises one or more miRNA target sequence. In still another embodiment, the miRNA target sequence are targets for miRNA present in off-target cells. In still another embodiment, the polynucleotide comprises a sequence encoding a polypeptide that converts a prodrug to a toxic drug.

The disclosure provides a plasmid comprising a sequence that produces a polynucleotide that is encapsulated into a viral capsid. In one embodiment, the polynucleotide comprises RNA. In another embodiment, the polynucleotide is derived from a gamma retrovirus. In still another embodiment, the polynucleotide comprises long terminal repeats at the 5′ and 3′ end. In yet another embodiment, the polynucleotide comprises a coding sequence for a CAR. In yet another embodiment, the polynucleotide comprises one or more miRNA target sequence. In still another embodiment, the miRNA target sequence are targets for miRNA present in off-target cells, but not in target cells, nor in cells used to make infectious vectors. In still another embodiment, the polynucleotide comprises a sequence encoding a polypeptide that converts a prodrug to a toxic drug.

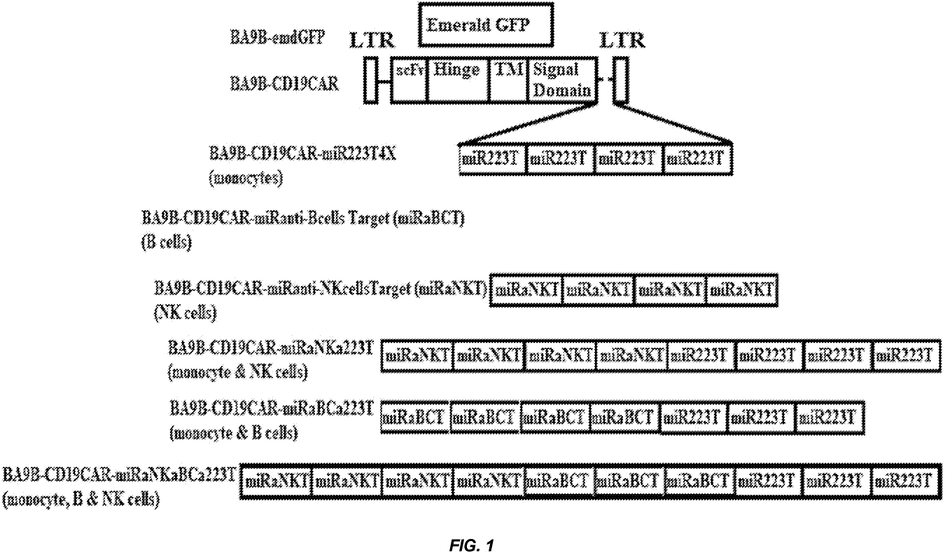

The disclosure provide a viral polynucleotide construct comprising from 5′ to 3′, an “R-U5” domain from a gammaretrovirus operably linked to a coding sequence for a binding domain operably linked to a hinge/linker coding sequence operably linked to a transmembrane domain coding sequence operably linked to a signaling domain coding sequence followed by one or more miRNA target sequences (miR-TS; miR target cassette) followed by a “U3-R″ domain from a gammaretrovirus. In some embodiments, the viral RNA can include a coding sequence for a kill switch operably linked to an IRES. In some embodiments, the IRES-Kill switch can be upstream or downstream (5′ or 3′) to the miRNA cassette. The polynucleotide sequence can be schematically presented as (see also FIG. 1): R-U5-Binding domain-hinge/linker-TM domain-signaling domain-miRNAtarget-U3-R

In one embodiment, the R-U5 domain can comprise a sequence that is at least 80-100% identical to the sequence:

GCGCCAGUCCUCCGAUUGACUGAGUCGCCCGGGUACCCGUGUAUCCAAUA
AACCCUCUUGCAGUUGCAUCCGACUUGUGGUCUCGCUGUUCCUUGGGAGG
GUCUCCUCUGAGUGAUUGACUACCCGUCAGCGGGGGUCUUUCAUU (SEQ
ID NO:25 from nucleotide 1 to 145)

In one embodiment, the R-U5-packaging domains can comprise a sequence that is at least 80-100% identical to the sequence:

GCGCCAGUCCUCCGAUUGACUGAGUCGCCCGGGUACCCGUGUAUCCAAUA
AACCCUCUUGCAGUUGCAUCCGACUUGUGGUCUCGCUGUUCCUUGGGAGG
GUCUCCUCUGAGUGAUUGACUACCCGUCAGCGGGGGUCUUUCAUUUGGGG
GCUCGUCCGGGAUCGGGAGACCCCUGCCCAGGGACCACCGACCCACCACC
GGGAGGUAAGCUGGCCAGCAACUUAUCUGUGUCUGUCCGAUUGUCUAGUG
UCUAUGACUGAUUUUAUGCGCCUGCGUCGGUACUAGUUAGCUAACUAGCU
CUGUAUCUGGCGGACCCGUGGUGGAACUGACGAGUUCGGAACACCCGGCC
GCAACCCUGGGAGACGUCCCAGGGACUUCGGGGGCCGUUUUUGUGGCCCG
ACCUGAGUCCAAAAAUCCCGAUCGUUUUGGACUCUUUGGUGCACCCCCCU
UAGAGGAGGGAUAUGUGGUUCUGGUAGGAGACGAGAACCUAAAACAGUUC
CCGCCUCCGUCUGAAUUUUUGCUUUCGGUUUGGGACCGAAGCCGCGCCGC
GCGUCUUGUCUGCUGCAGCAUCGUUCUGUGUUGUCUCUGUCUGACUGUGU
UUCUGUAUUUGUCUGAGAAUUAAGGCCAGACUGUUACCACUCCCUGAAGU
UUGACCUUAGGUCACUGGAAAGAUGUCGAGCGGAUCGCUCACAACCAGUC
GGUAGAUGUCAAGAAGAGACGUUGGGUUACCUUCUGCUCUGCAGAAUGGC
CAACCUUUAACGUCGGAUGGCCGCGAGACGGCACCUUUAACCGAGACCUC
AUCACCCAGGUUAAGAUCAAGGUCUUUUCACCUGGCCCGCAUGGACACCC
AGACCAGGUCCCCUACAUCGUGACCUGGGAAGCCUUGGCUUUUGACCCCC
CUCCCUGGGUCAAGCCCUUUGUACACCCUAAGCCUCCGCCUCCUCUUCCU
CCAUCCGCCCCGUCUCUCCCCCUUGAACCUCCUCGUUCGACCCCGCCUCG
AUCCUCCCUUUAUCCAGCCCUCACUCCUUCUCUAGGCGCCGGAAUUAAUU
CUCGA (SEQ ID NO:25 nucleotide 1 to1055)

In one embodiment, the U3-R domain can comprise a sequence that is at least 80-100% identical to the sequence:

UGAAAGACCCCACCUGUAGGUUUGGCAAGCUAGCUUAAGUAACGCCAUUU
UGCAAGGCAUGGAAAAAUACAUAACUGAGAAUAGAGAAGUUCAGAUCAAG
GUCAGGAACAGAUGGAACAGCUGAAUAUGGGCCAAACAGGAUAUCUGUGG
UAAGCAGUUCCUGCCCCGGCUCAGGGCCAAGAACAGAUGGAACAGCUGAA
UAUGGGCCAAACAGGAUAUCUGUGGUAAGCAGUUCCUGCCCCGGCUCAGG
GCCAAGAACAGAUGGUCCCCAGAUGCGGUCCAGCCCUCAGCAGUUUCUAG
AGAACCAUCAGAUGUUUCCAGGGUGCCCCAAGGACCUGAAAUGACCCUGU
GCCUUAUUUGAACUAACCAAUCAGUUCGCUUCUCGCUUCUGUUCGCGCGC
UUCUGCUCCCCGAGCUCAAUAAAAGAGCCCACAACCCCUCACUCGGCGCG
CCAGUCCUCCGAUUGACUGAGUCGCCCGGGUACCCGUGUAUCCAAUAAAC
CCUCUUGCAGUUGCA (SEQ ID NO:25 fromnucleotide 5537
to 6051)

It will be recognized by one of skill in the art that a DNA plasmid sequence of the R-U5 and U3-R sequences will have “U” replaced with “T”.

The binding domain of the “CAR” can be any sequence that encodes a polypeptide that binds to a desired target antigen. For example, the binding domain can be an antibody fragment such as an scFv directed to a desired target antigen (see, e.g., Table 1). Sequences encoding various binding domains to the targets set forth in Table 1 are known in the art and published in numerous applications. The CARs of the disclosure are modular in nature and thus different “binding domains” can be attached depending upon the desired target.

As described above, a ‘hinge’ or linker coding sequence can be operably linked to the binding domain of the CAR. In some instances the ‘hinge’ is optional and the binding domain can be directly linked to the transmembrane domain coding sequence. In another embodiment, the binding domain and transmembrane domain are separated by a minimal peptide coding sequence or spacer. Various hinge domains and spacers are known in the art and described herein.

The miR targeting sequence or cassette will typically comprise a target for an miRNA molecules that would inhibiting expression of a polynucleotide of the viral construct. For example, the miR target sequence will be typically bind an miRNA that is expressed in a tissue or cell where expression of, e.g., a CAR is undesirable or unwanted, but the miRNA is not expressed in target cells nor in vector producer cells where expression from the viral construct is desired. When such sequences or miRNA are not already known, they can be readily identified and characterized by making total RNA and performing deep bulk sequencing on such samples, from several examples of target tissues for which expression is not wanted (e.g., a tumor) and from several examples of cells where expression is desirable or needed (e.g., T cells) then using bioinformatic techniques known to those skilled in the art, candidate miRNAs and corresponding targets for further testing are identified. Additionally, using the disclosure one of skill in the art can readily identify a binding domain for treating a particular cancer or disease, as well as an miRNA targeting sequence that would prevent expression of the CAR in undesirable tissues and/or cells and suitable ‘hinge’, ‘transmembrane domain’ and intracellular domains.

In some embodiments, a vector construct of the disclosure will include a kill switch as a further safety mechanism, such that expression of the vector construct will result in expression of, e.g., a suicide gene (e.g., a polypeptide having thymidine kinase (TK) or cytosine deaminase (CD) activity). In instances where a subject, cells or tissues have developed unwanted vector expression, the subject, tissue or cell is contacted with a pro-drug (e.g., 5-flurocytosine) such that the cells expressing, e.g., a polypeptide having cytosine deaminase activity are contacted by the 5-FC wherein the 5-FC is converted to cytotoxic 5-FU at the site of the kill-switch’s expression thereby killing the vector-infected cell.

In one embodiment, the disclosure provides a retroviral vector comprising a gag polypeptide, a pol polypeptide and an env polypeptide and a retroviral polynucleotide contained within the capsid of the retroviral vector. The retroviral polynucleotide comprising from 5′ to 3′: R-U5-Binding domain-hinge/linker-TM domain-signaling domain-miRNAtarget-U3-R. In one embodiment, the retroviral polynucleotide comprises an R-U5 nucleic acid sequence having at least 80%, 85%, 87%, 90%, 92%, 95%, 98%, 99% or 100% identity to SEQ ID NO:25 from nucleotide 1 to about nucleotide 145 (e.g., about nucleotide 140, 141, 142, 143, 144, 145, 126, 147, 148, 149 or 150). In a further embodiment, the retroviral polynucleotide comprises a chimeric antigen receptor coding sequence 3′ to the R-U5 domain. In one embodiment, the CAR coding sequence comprises a coding sequence for an antigen binding domain (e.g., an scFv to CD19). In some embodiments, the binding domain coding sequence can be preceded by a signal sequence. In a further embodiment, the binding domain coding sequence is followed by an optional linker/spacer domain sequence. In still a further embodiment, the binding domain coding sequence an optional spacer/linker coding sequence is followed by a nucleic acid sequence encoding a transmembrane domain. In a further embodiment, the transmembrane coding sequence is followed by a nucleic acid sequence encoding a cytoplasmic signaling domain. In another embodiment, the retroviral polynucleotide can comprise an optional kill switch domain coding sequence. The optional kill switch coding domain comprises an IRES operably linked to a coding sequence for a polypeptide that converts a prodrug to a cytotoxic drug. In one embodiment, the polypeptide is thymidine kinase (TKO) or cytosine deaminase (CD). In a further embodiment, the retroviral polynucleotide comprises at least one, typically a plurality of the same of different miRNA targeting sequences. The miRNA targeting sequences are 3′ to the CAR domain. The retroviral polynucleotide further includes a U3-R domain at the 3′ end of the polynucleotide. In one embodiment, the U3-R domain comprises a sequence that is at least 80%, 85%, 87%, 90%, 92%, 95%, 98%, 99% or 100% identity to SEQ ID NO:25 from about nucleotide 5537 to about 6051. In some embodiments of any of the foregoing, the domains can be separated by small spacer sequences of about 2-20 nucleotides that can be intentional or artifacts of cloning.

The disclosure also provides a plasmid sequence that when expressed in a suitable host cell produces the retroviral vectors of the disclosure.

The disclosure provides retroviral vectors comprising a recombinant viral genome having a R-U5 domain, a packaging domain, a CAR domain and a miRNA detargeting domain (e.g., a miRNA targeting domain). The recombinant viral genome can further comprise a kill switch comprising a coding sequence for a suicide gene (e.g., a gene that produced a polypeptide that has TKO or CD activity). The CAR domain can comprise a 1^{st}, 2^{nd} or 3^{rd} generation CAR construct (as are known in the art) which when expressed in a desired immune cells is capable of binding to a target antigen. The retroviral vector of the disclosure comprises retroviral capsid comprising an envelope that can infect mammalian cells and deliver the recombinant viral genome into the mammalian cell. The retroviral vector can be used to transform cell in vivo thus eliminating the need for ex vivo isolation of cells as is performed in current adoptive cell therapy. The miRNA detargeting domain comprises targeting sequences that can be bound by miRNA produced in cells where expression of the viral genome is undesirable. The binding of the miRNA in these cells binds the miRNA targeting sequence and prevents expression of the viral genome in the cell.

The disclosure also provides methods of treating a subject with cancer. The method comprises inducing, in vivo, expression of a CAR, without ex vivo immune cell manipulation. The method can include identifying a target antigen specific to a disease or disorder to be treated. Constructing a chimeric antigen receptor having a binding domain that targets the antigen specific for the disease or disorder. Cloning the CAR coding sequence into a vector of the disclosure. Generating a viral construct containing a polynucleotide coding for the CAR construct and administering the viral construct such that the subject’s immune cells are transduced in vivo to express the CAR.

A vector of the disclosure can be generated as described herein, purified and prepared in a pharmaceutical formulation for administration to a subject.

In another embodiment, the disclosure provides a method of mobilizing stem cells (e.g., hematopoietic stem cells) in a subject that is planning to, undergoing or has undergone therapy with a vector of the disclosure.

The disclosure provides a number of ways to mobilize hematopoietic stem cells. Hematopoietic stem and progenitor cells (HSPCs) reside in distinct niches within the bone marrow environment, populated by several cell types such as osteoblasts, reticular/mesenchymal cells, endothelial cells, macrophages, and megakaryocytes. These niche cells serve a regulatory function, limiting the entry of HSPCs into the cell cycle, and ensuring lifelong repopulation of the hematopoietic system by a limited number of HSPCs that can maintain and regenerate the HSPC pool. HSPCs can be mobilized from these niches into the blood, and this mobilization can be induced by growth factors, drugs, antibodies, etc. Once mobilized, HSPCs travel through the bloodstream and can home back to sites of hematopoiesis. The disclosure provides several different ways that effectively do this to allow transduction of hematopoietic stem cells (HSC) in the blood before returning to the relatively inaccessible marrow niche.

The disclosure also provides methods of targeting retroviral vectors to HSC using envelopes on a retroviral capsid having moieties designed to bind to epitopes displayed on the surface of these cells.

Osteoblasts regulate the quiescence or proliferation of HSPCs through their expression of soluble and membrane-localized factors. For example, osteoblasts produce hematopoietic growth factors like granulocyte colony stimulating factor (G-CSF) and hepatocyte growth factor (HGF) upon contact with CD34+ HSPC or stimulation with either parathyroid hormone (PTH) or the locally produced PTH-related protein (PTHrP) through the PTH/PTHrP receptor (PPR). Moreover, bone marrow stromal cells cultured in the presence of PTH gained capacity to maintain long-term bone marrow-initiating cells (LTC-IC), and application of PTH increases HSPCs with bone marrow-repopulating activity. Thus, PTH can regulate HSPC proliferation by influencing relevant growth signals for HSPCs on osteoblastic stromal cells (Calvi et al., 2003).

Osteoblasts supporting HSPCs further have a distinct phenotype of N-cadherin+ CD45-, and are regulated by bone morphogenetic protein (BMP). These osteoblasts express chemokines such as C-X-C motif chemokine-12 (CXCL12; also known as Stromal-derived factor-1 (SDF1)), as well as stem cell factor (SCF), interleukin-6 (IL-6) and the Notch ligand, Jagged 1 (Jag1). Increasing Notch signaling in HSPCs, for example, through PTH/PTHrP activation of PRR of Jag1 in osteoblasts, increases numbers of HSPCs without affecting mature hematopoietic cells, whereas blocking Notch signaling by gamma-secretase inhibition of Notch activation decreases numbers of long-term repopulating HSPCs (Calvi et al., 2003; Stier et al., 2002).

Osteoblasts further express Angiopoietin-1, which binds to the Tie2 receptor on HSPCs to support HSPCs quiescence, adhesion of HSPCs to bone areas, and maintenance of HSPCs (Arai et al., 2004). Osteoblasts also express thrombopoietin (TPO), which activates the MPL receptor that is expressed on quiescent HSPCs in the bone marrow; TPO/MPL interaction upregulates betal-integrin and cyclin-dependent kinase inhibitors in HSPCs and thereby induce quiescence of HSPC, whereas inhibition of the TPO/MPL pathway with anti-MPL-neutralizing antibody reduces the number of quiescent HSPCs (Yoshihara et al., 2007; Qian et al., 2007).

Another factor in the regulation of primitive HSPC proliferation in the osteoblastic niche is Osteopontin, which restricts primitive cell expansion in the bone marrow niche. Osteopontin is produced by osteoblasts, and primitive HSPC demonstrate specific adhesion to Osteopontin in vitro via beta1 integrin (Nilsson et al., 2005). Deficiency or inhibition of Osteopontin results in significantly increased stromal Jag1 and Notch1 receptor expression on human CD34+ HSPCs, resulting in increased numbers of long-term repopulating HSPC (Stier et al., 2005; Iwata et al., 2004).

The bone marrow stroma further contains fibroblast-like cells which are part of the adherent fraction of bone marrow cells and which forms a hematopoiesis-supporting adherent layer when bone marrow is placed into long-term culture conditions. These fibroblastic mesenchymal cells can differentiate into various lineages such as osteoblasts, chondrocytes, or adipocytes, and have been variously termed as marrow stromal cells, mesenchymal stem cells (MSC), adventitial reticular cells (ARCs), and STRO-1 cells. These cells can be identified and isolated by the use of antibodies recognizing cell surface markers such as STRO-1, SH2, SH3, SH4, Nestin, platelet-derived growth factor receptor-a (PDGFRa), CD51, CD146 and are negative for CD45, CD31, and Ter119 (Simmons et al., 1994; Sacchetti et al., 2007; Mendez-Ferrer et al., 2010; Pnho et al., 2013). Arteriolar perivascular cells which express the mesenchymal cell marker NG-2 (Cspg4) also maintain HSPC quiescence (Kunisaki et al., 2013).

In particular, Nestin is a marker for a small subpopulation of non-hematopoietic MSCs, which are spatially associated with HSPCs and adrenergic nerve fibers (Mendez-Ferrer et al., 2010). Most HSPCs are in close contact with stromal cells expressing high amounts of the chemokine CXCL12, termed ‘CXCL12-abundant reticular’ cells, which either surround sinusoidal endothelial cells or are located near the endosteum (Sugiyama et al., 2006). CXCL12 expression is >50-fold higher in Nestin+ than in Nestin- stromal cells, and 10-fold higher than in primary osteoblasts. Depletion of Nestin+ MSCs results in a 50% reduction of immature HSPCs and a 90% reduction of HSPC homing to the bone marrow. Moreover, administration of PTH induced proliferation of Nestin+ MSCs, their differentiation into osteoblasts, and an increase of the HSPC pool (Mendez-Ferrer et al., 2010; Adams et al., 2007).

Furthermore, compared to Nestin- MSCs, Nestin+ MSCs highly express HSPC maintenance genes SCF/c-kit Ligand, IL-7, and vascular cell adhesion molecule-1 (VCAM-1) and, as a counter-regulator of HSPC maintenance, osteopontin. Also, CXCL12-abundant reticular cells are innervated by the sympathetic nervous system, as confirmed by high expression of connexins 43 and 45 in Nestin+ MSCs, indicating their electromechanical coupling with ?-adrenergic nerve terminals, and administration of a ?3 adrenergic receptor agonist enhances mobilization of HSPCs (Katayama et al., 2006).

Endothelial cells also contribute to HSPC niche signals. Imaging of bone marrow vascular structures showed that 85% of long-term repopulating HSPCs were within 10 µm of a sinusoidal blood vessel and in contact with leptin receptor+ and CXCL12-high stromal niche cells (Acar et al., 2015). Recent work also demonstrated that more than 94% of HSPCs in the bone marrow marked by expression of HoxB5 are found in an abluminal position of the vessel and are directly attached to VE-cadherin (Cdh5)-expressing endothelial cells (Chen et al., 2016).

Direct cellular contact of HSPCs with human sinusoidal endothelial cells increases repopulation potential and self-renewal of HSPCs through the Notch ligands Jagged 1 (Jag1), Jag2, Delta-like ligand 4 (D114), D111, and endothelium-targeted synthetic fusion proteins derived from D111 and other Notch ligands can enhance HSPC regeneration (Butler et al., 2010; Tian et al., 2013). Although Notch signaling is not essential for homeostasis of adult HSPCs, Notch-ligand adhesive interaction maintains HSC quiescence and niche retention, and it has recently been reported that Notch2 blockade (but not Notch1 blockade) sensitizes HSPCs to mobilization stimuli and leads to enhanced egress from marrow to the peripheral blood (Wang et al., 2017).

On the other hand, disruption of VE-cadherin-dependent and vascular-endothelial growth factor receptor 2 (VEGFR2)-dependent angiogenic signaling pathways, which are significant for endothelial cell survival, by monoclonal antibodies for 3 days also has been reported to disrupt Notch signaling in hematopoietic cells, resulting in decreased frequency of HSPCs, and inducing differentiation (Butler et al., 2010). During chemotherapy, sinusoidal vessels including arterioles and sinusoidal endothelial cells are dose-dependently damaged, and VEGFR2 is essential for their regeneration and HSPC reconstitution (Hooper et al., 2009).

Other factors regulating HSPC proliferation include Stem Cell Factor (SCF) in endothelial cells and leptin receptor-expressing perivascular cells (Ding et al., 2012). The heparin-binding growth factor pleiotrophin, which is expressed and secreted by bone marrow sinusoidal endothelial cells, has also been shown to regulate hematopoietic stem cell self-renewal and retention (Himburg et al., 2012). Moreover, deletion of the endothelial cell-specific adhesion molecule, E-selectin induces increased HSPC quiescence, suggesting that endothelial cells can also regulate HSPC proliferation (Winkler et al., 2012).

HSPCs are also found in contact with bone marrow macrophages, which support osteoblast survival and retention of HSPCs in their niche (Chow et al. 2011; Christopher et al. 2011). Depletion of CD169+ (Siglec1) macrophages leads to decreased retention of HSPCs in the mesenchymal (ARC) niche in the bone marrow, and consequently HSPCs are mobilized in the bloodstream (Chow et al. 2011). Furthermore, G-CSF induces depletion of endosteal macrophages, which leads to suppression of osteoblast function and HSPC mobilization (Winkler et al. 2010).

HSPCs are also found in direct contact with megakaryocytes, which also contribute to the HSPC niche. Megakaryocytes normally secrete cell cycle regulators such as thrombopoietin (TPO), transforming growth factor ?1 (TGF-?1), and chemokine C-X-C motif ligand-4 (CXCL4), which keep HSPCs in the G0 phase of the cell cycle (Nakamura-Ishizu et al. 2014; Bruns et al. 2014; Zhao et al. 2014). In fact, TGF-β1 is reported to be expressed more highly in megakaryocytes than any other stromal cell type in the bone marrow, including osteoblasts, endothelial cells, Nestin+ perivascular cells, and CXCL12-abundant reticular cells; however, under the chemotoxic stress of chemotherapeutic drugs such as 5-fluoruracil (5-FU), megakaryocytes secrete fibroblast growth factor 1 (FGF-1) and down-regulate TGF-β1, stimulating the expansion of HSPCs (Zhao et al. 2014).

The mobilization of HSPCs is a multifactorial process and is regulated at the level of the bone marrow microenvironment through modulation of the interaction between HSPC and bone marrow stroma. Adhesion molecules, paracrine cytokines, and chemokines have been implicated in this interaction. The main factors that anchor HSPCs in the niche, and/or induce their quiescence are vascular cell adhesion molecule (VCAM)-1, CD44, hematopoietic growth factors, e.g. stem cell factor (SCF) and FLT3 Ligand, chemokines including CXCL12, growth-regulated protein beta and IL-8, proteases, peptides, and other chemical transmitters such as nucleotides. Mobilization is initiated by disengagement from stromal adhesion, followed by directed migration toward bone marrow sinuses, and subsequent egress through the basement membrane and the endothelial layer.

HSPC exhibit a wide range of cell adhesion molecules (CAM), with ligands found on bone marrow stromal cells. The role and contribution of the many CAM-ligand pairs in the mobilization and homing process is largely unknown. However, the expression of several leukocyte adhesion molecules, including the integrins LFA-1 (Lymphocyte function-associated antigen 1) and VLA-4 (Very Late Antigen-4 or integrin α4β1), is reduced on circulating progenitors when compared with progenitor cells resident in the bone marrow. The respective ligands (Intercellular Adhesion Molecule-1 or ICAM-1, and Vascular Cell Adhesion Molecule-1 or VCAM-1) for these adhesion molecules are found on bone marrow endothelial and stromal cells. Primarily, the VCAM-1 protein is an endothelial ligand for VLA-4 (α4β1), and binds weakly to LPAM (Lymphocyte Peyer patch adhesion molecule or integrin α4β7) . More than 90 percent of all purified peripheral blood CD34+ cells express VLA-4 (α4β1) integrins, whereas only 10 to 15 percent express VLA-5 (integrin α5β1 or fibronectin receptor). The VLA-4 (α4β1) integrin alone influences adhesion whereas VLA-4 (α4β1) and VLA-5 (α5β1) both mediate chemotaxis of clonogenic CD34+ progenitor cells on recombinant fibronectin (Carstanjen et al. 2005).

The release of HSPCs from stromal cells within the bone marrow is effected by proteolytic degradation of VCAM-1 by elastase and cathepsin G, and of CXCL12 by neutrophil proteases (Levesque et al. 2002). Also, shedding of membrane-bound SCF by matrix metalloproteinase 9 (MMP-9) has been found to contribute to HSPC mobilization (Heissig et al. 2002). These discoveries pointed to a common ‘end pathway’ in HSPC mobilization, after stimulation with G-SCF, but also after stimulation with other stimuli such as chemokines or chemotherapy.

In early studies, antibodies to VLA-4 and VCAM were shown to mobilize progenitor cells and/or inhibit their homing to the bone marrow in non-human primates (Papayannopoulou et al. 1995; Papayannopoulou and Nakamoto 1993). Subsequently, the significance of not only VLA-4 / VCAM-1 interaction, but also VLA-5 (?5?1; fibronectin receptor) / fibronectin and CD44 / hyaluronan / osteopontin interactions between HSPCs and stromal cells for retention of HSPCs in the bone marrow niche has been indicated by studies using function-blocking anti-VLA-5 and anti-CD44 antibodies, all resulting in liberation of HSPCs and their mobilization into the blood (Vermeulen et al. 1998; van der Loo et al. 1998). Importantly, antibodies against VLA-4 (α4β1) and VLA-5 (α5β1) independently reduce the repopulation of bone marrow after transplantation of human peripheral blood CD34+ cells (Carstanjen et al. 2005).

Natalizumab is a humanized monoclonal anti-VLA-4 antibody which is FDA approved for the treatment of patients with multiple sclerosis and Crohn’s disease. Serial measurements in patients receiving their natalizumab infusion therapy for multiple sclerosis showed a significant increase in circulating CD34+ cells of approximately 3-fold (Zohren et al. 2008). In the context of hematopoietic stem cell transplantation and stem cell diseases, the use of natalizumab alone or in combination with either cytotoxic drugs or other antibodies might be a new modality for stem cell mobilization (Neumann, Zohren, and Haas 2009).

Low expression or reduced avidity of the adhesion molecules on HSPC may also facilitate their mobilization from the bone marrow. For example, it has been shown that specific cytokines such as interleukin (IL)-3, granulocyte-macrophage CSF (GM-CSF), and KIT ligand (KL) are capable of modifying the function of VLA-4 and VLA-5 expressed on CD34+ cells and thereby modulating adhesion to fibronectin (Levesque et al. 1995). Conversely, after Stem Cell Factor (SCF) stimulation, expression of VLA-4 and VLA-5 on CD34+ HSPC has been reported to increase by approximately 2-fold and 4- to 10-fold, respectively, resulting in increased adhesion to fibronectin after stimulation with SCF (Hart et al. 2004).

In addition, mutually exclusive distribution of EPHB4 receptors in bone marrow sinusoids and ephrin B2 ligands in hematopoietic cells indicates the role of interactions of these molecules in HSPC mobilization. Blockade of the EPHB4/ephrin B2 signaling pathway in mice was shown to reduce mobilization of HSPCs and other myeloid cells to the circulation (Kwak et al. 2016).

Pharmaceutical compositions of the disclosure may comprise a viral vector, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure can be formulated for intravenous administration. The composition may further comprise a secondary active agent (e.g., an anticancer, antiviral or antibiotic agent).

Pharmaceutical compositions of the disclosure may be administered in a manner appropriate to the disease to be treated (or prevented). The quantity and frequency of administration will be determined by such factors as the condition of the patient, and the type and severity of the patient’s disease. When “an immunologically effective amount,” “an anti-tumor effective amount,” “a tumor-inhibiting effective amount,” or “therapeutic amount” or “anti-infective” is indicated, the amount of the compositions of the disclosure to be administered can be determined by a physician with consideration of individual differences in age, weight, tumor size, extent of infection or metastasis, and condition of the patient (subject) as the case may be. It can generally be stated that a pharmaceutical composition is dosed at an amount sufficient to cause the transduction of immune effector cells (e.g., T cells, NK cells) sufficient to treat the disease or disorder. In one embodiment, a pharmaceutical composition of the disclosure comprises vector at 10³ to 10¹¹ transforming units/dose (e.g., 10³, 10⁴, 10⁵, 10⁶, 10⁷, 10⁸, 10⁹, 10¹⁰, 10¹¹ or any value between any of the foregoing two values). The dose may be administered one time to several times per day and may be administered for consecutive days, weeks or months as necessary to induce immune effector cells in vivo. The pharmaceutical composition comprising vectors of the disclosure can be administered by using infusion techniques that are commonly known in immunotherapy (see, e.g., Rosenberg et al., New Eng. J. of Med. 319:1676, 1988).

The disclosure is further described by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### EXAMPLES

### Example 1

### In Vitro Testing of Selective Transgene Expression From Recombinant Retroviral Vectors Containing miRNA Target Sequences

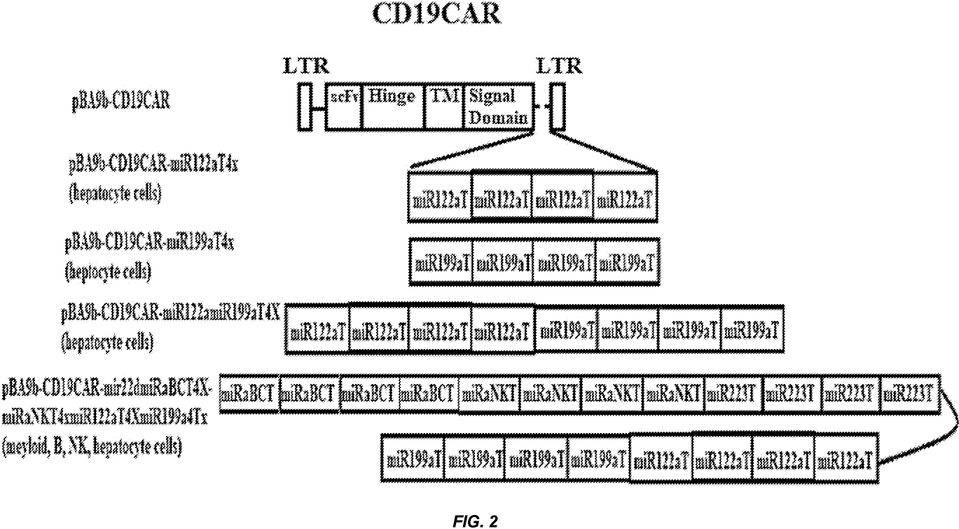

This experiment is conducted with an RNV that contains a transgene that includes a microRNA target sequence within the transcribed coding RNA for the transgene. Infectious vector is prepared by any of the methods described in Examples 2-6. As a convention the plasmid name has a small “p” preceding the full name and the corresponding infectious vector has the same name without the “p” or with a preceding or following “v”. Thus, pBA9-9b-hCD19 -miRT223T4X means the plasmid (e.g., construct 7) carrying the viral backbone from pBA-9b (SEQ ID NO:1 wherein “U” can be “T”) containing a coding sequence for an anti-human CD19CAR and 4 copies of a target sequence for miR223; BA-9b-hCD19-miRT223T4X or vBA-9b-hCD19-miRT223T4X or BA-9b-hCD19-miRT223T4X(V) represents the corresponding infectious vector. Cells that express the cognate microRNA will degrade RNA that include the miRNA target sequence thereby limiting expression of the transgene in that cell type. Transgenes may include 1 or more copies of each microRNA target sequence. In this example the transgene encodes for a CD19 chimeric antigen receptor without and with 4 copies of each miRNA target sequence at the 3′ end of the transcript. In one embodiment the microRNA target sequence codes for 4 repeats of the miR223 target (“miR223T(4X)”) to reduce transgene expression in transduced monocytes (FIG. 1; construct 7; SEQ ID NO:2). In one embodiment the microRNA target sequence codes for a B cell (lymphoma) specific miRaBCT(4X) to reduce transgene expression in transduced B cells (FIG. 1). In one embodiment the microRNA target sequence codes for miRaNKT(4X) to reduce transgene expression in transduced NK cells (FIG. 1). In one embodiment the microRNA target sequence codes for miRaBCT(4X) and miR223T(4X) to reduce transgene expression in transduced B cells and monocytes (FIG. 1; construct 5). In one embodiment the microRNA target sequence codes for miRaNKT(4X) and miR223T(4X) to reduce transgene expression in transduced B cells and monocytes (FIG. 1). In one embodiment the microRNA target sequence codes for miRaNKT(4X), miRaNKT(4X) and miR223T(4X) to reduce transgene expression in transduced B cells and monocytes (FIG. 1). In another embodiment, RNV may also contain miRNA target sequences in the transgene UTR that result in transcript degradation in hepatocytes. Addition of miRNA122aT(4X) and miR199aT(4X) sequences as single targets or combined to the UTR reduces transgene expression in the liver (FIG. 2; construct 36, 37, 38). These liver detargeting miRNAs may be combined with components from FIG. 1 including a combination where all 5 miRNA target sequences are encoded in the RNV transgene UTR (FIG. 2).

An RNA viral polynucleotide (vRNA genome) of the disclosure is provided in SEQ ID NO:1; Bold/underlined portion identifies multiple cloning site:

GCGCCAGUCCUCCGAUUGACUGAGUCGCCCGGGUACCCGUGUAUCCAAUA
AACCCUCUUGCAGUUGCAUCCGACUUGUGGUCUCGCUGUUCCUUGGGAGG
GUCUCCUCUGAGUGAUUGACUACCCGUCAGCGGGGGUCUUUCAUUUGGGG
GCUCGUCCGGGAUCGGGAGACCCCUGCCCAGGGACCACCGACCCACCACC
GGGAGGUAAGCUGGCCAGCAACUUAUCUGUGUCUGUCCGAUUGUCUAGUG
UCUAUGACUGAUUUUAUGCGCCUGCGUCGGUACUAGUUAGCUAACUAGCU
CUGUAUCUGGCGGACCCGUGGUGGAACUGACGAGUUCGGAACACCCGGCC
GCAACCCUGGGAGACGUCCCAGGGACUUCGGGGGCCGUUUUUGUGGCCCG
ACCUGAGUCCAAAAAUCCCGAUCGUUUUGGACUCUUUGGUGCACCCCCCU
UAGAGGAGGGAUAUGUGGUUCUGGUAGGAGACGAGAACCUAAAACAGUUC
CCGCCUCCGUCUGAAUUUUUGCUUUCGGUUUGGGACCGAAGCCGCGCCGC
GCGUCUUGUCUGCUGCAGCAUCGUUCUGUGUUGUCUCUGUCUGACUGUGU
UUCUGUAUUUGUCUGAGAAUUAAGGCCAGACUGUUACCACUCCCUGAAGU
UUGACCUUAGGUCACUGGAAAGAUGUCGAGCGGAUCGCUCACAACCAGUC
GGUAGAUGUCAAGAAGAGACGUUGGGUUACCUUCUGCUCUGCAGAAUGGC
CAACCUUUAACGUCGGAUGGCCGCGAGACGGCACCUUUAACCGAGACCUC
AUCACCCAGGUUAAGAUCAAGGUCUUUUCACCUGGCCCGCAUGGACACCC
AGACCAGGUCCCCUACAUCGUGACCUGGGAAGCCUUGGCUUUUGACCCCC
CUCCCUGGGUCAAGCCCUUUGUACACCCUAAGCCUCCGCCUCCUCUUCCU
CCAUCCGCCCCGUCUCUCCCCCUUGAACCUCCUCGUUCGACCCCGCCUCG
AUCCUCCCUUUAUCCAGCCCUCACUCCUUCUCUAGGCGCCGGAAUUAAUU CUCGAGGGGCCCAGAUCUGCGGCCGCUCGCGAGUCGAC AAGCUUGGAUCC
AUCGAUAAAAUAAAAGAUUUUAUUUAGUCUCCAGAAAAAGGGGGGAAUGA
AAGACCCCACCUGUAGGUUUGGCAAGCUAGCUUAAGUAACGCCAUUUUGC
AAGGCAUGGAAAAAUACAUAACUGAGAAUAGAGAAGUUCAGAUCAAGGUC
AGGAACAGAUGGAACAGCUGAAUAUGGGCCAAACAGGAUAUCUGUGGUAA
GCAGUUCCUGCCCCGGCUCAGGGCCAAGAACAGAUGGAACAGCUGAAUAU
GGGCCAAACAGGAUAUCUGUGGUAAGCAGUUCCUGCCCCGGCUCAGGGCC
AAGAACAGAUGGUCCCCAGAUGCGGUCCAGCCCUCAGCAGUUUCUAGAGA
ACCAUCAGAUGUUUCCAGGGUGCCCCAAGGACCUGAAAUGACCCUGUGCC
UUAUUUGAACUAACCAAUCAGUUCGCUUCUCGCUUCUGUUCGCGCGCUUC
UGCUCCCCGAGCUCAAUAAAAGAGCCCACAACCCCUCACUCGGCGCGCCA
GUCCUCCGAUUGACUGAGUCGCCCGGGUACCCGUGUAUCCAAUAAACCCU
CUUGCAGUUGCA

A CAR construct and (without or without additional domains, e.g., miRNA targeting domain, kill switch domain etc.) can be cloned into the multiple cloning site.

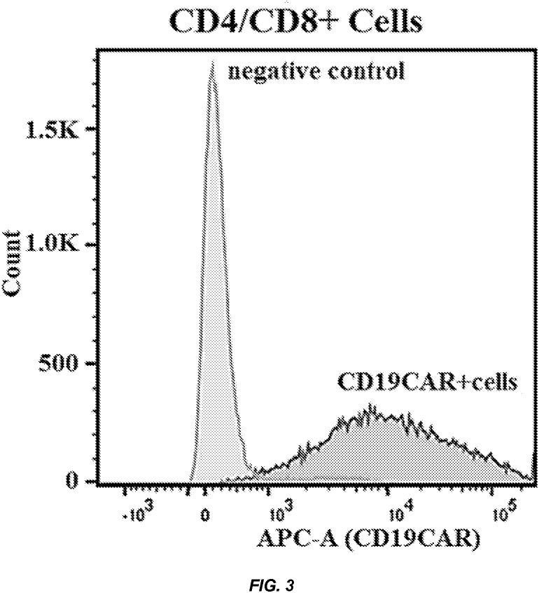

To confirm miRNA target sequence specificity and function in varying cell types, RNV containing emerald GFP is used in place of CD19CAR to look at transgene expression across PMBC cell types and varying cell lines. Vector BA9B-emdGFP-containing miR variants described in FIG. 1 was used to transduce cell lines: Jurkat (T cell); TALL-104 (T cell); Raji (B); NALM-6 (B); THP-1 (monocyte); U937 (monocyte) and NK-92 (NK). As positive control, HT1080 cells are transduced with a 3^{rd} generation SIN-lentiviral vector expressing a single or varying combinations of precursor miRNAs along with puromycin. Post-transduction HT1080s are selected with puromycin (6ug/mL) for two weeks, before RNV transduction that encodes the desired transgene with miRNA target sequences in the UTR. This ensures at least one cell line that is transduced will express the desired miRNA(s) to target the RNV transgene UTR for degradation. For example, in cells transduced with construct 5, only those cell lines that do not express the cognate miRNAs for the mRNA target sequences show eGFP expression as summarized in the Table 2. The same GFP expressing vector is used to transduce PBMCs with similar results showing that primarily it is only the T cells that express the GFP transgene at appreciable levels and frequency (FIG. 3).

TABLE 2

GFP expression after transduction with BA9B-CD19CAR-miRaBCT4X-miRaNKT4X-miR223T4X (predicted)

| | | | |
|---|---|---|---|
| Cell Line | Vector | %GFP+ cells | MFI of positive GFP cells |
| Jurkat | Construct 2: BA9B-emdGFP-miRaBCT4X-miRaNKT4X-miR223T4X | 70 | 78020 |
| TALL-104 | | 77 | 92248 |
| Raj i | | 3 | 2170 |
| NALM-6 | | 1 | 1132 |
| THP-1 | | 1 | 1005 |
| U937 | | 1 | 1792 |
| NK-92 | | 3 | 1444 |
| HT1080- miaBC; miRaNK; miR223; | SIN-Lenti with precursor miRNAs and puromycin | 7 | 2208 |
| miR122a; miR199a | | | |

In parallel, BA9B-CD19CAR-miRaBCT4X-miRaNKT4X-miR223T4X vector is used at 10 MOI to transduce PBMCs in a mix lymphocyte reaction in the presence of polybrene (4-8 ug/ml). One million PBMCs are seeded at 1e6/mL density in RPMI1640 medium supplemented with 10%FBS in a single well of a 24 well tissue culture plate. 24-48 hours post-transduction, cells are sampled and run on a cytometer to evaluate cell type specific expression of CD19CAR (FIG. 3). Only T cells which expressed CD4 or CD8 canonical markers show CD19CAR expression by flow cytometry while other cell types, including B cells, NK cells and monocytes, do not show CD19CAR expression. Also, reduced CD19+ B cells frequencies can be seen in cultures of PBMCs due to CD19CAR activity from T cells. Further, CD19CAR expressing T cells may be enriched during the performance of some in vitro assays described below.

For the CD19 CAR-specific activity of reprogrammed T cells, multiple short and long-term cellular assays are performed to measure CAR-mediated proliferation, cytokines production both intracellularly and in secretion and cytotoxicity against the Nalm6-CD19WT and Nalm6-CD19KO tumor cell lines. Tumor line Nalm6-CD19WT is purchased from ATCC and maintained in medium RPMI-1640 supplemented with 10% fetal bovine serum for the co-culture with reprogrammed T cells to test CD19 CAR activity. To measure CD19 CAR-non-specific activity of reprogrammed T cells, a CD19 antigen deficient variant of Nalm6 (Nalm6-CD19KO) is generated using CRISPR technology from the original Nalm6-CD19WT parental tumor line. Supernatants from the co-cultures of reprogrammed CD19 CAR-T cells with Nalm6 at different effectors to target (E:T) ratios of 16:1, 8:1, 4:1, 2:1, 1:1, 1:2, 1:4 and 1:8 are collected to measure secreted cytokines levels using Enzyme-linked Immunosorbent assay (ELISA) and flow cytometry using Biolegend’s Legendplex assay. Reprogrammed CAR-T cells secrete cytokines such as IL2, IFNγ and TNF only in the supernatants from culture with Nalm6-CD19WT not with Nalm6-CD19KO in a CD19 CAR-specific manner. Flow cytometry-based methods are also used to detect degranulation (CD107 mobilization) and intracellular cytokines on reprogrammed-CAR-expressing T cells after 4 hrs of short-term co-culture with NALM6-CD19WT and Nalm6-CD19KO cell lines separately. Reprogrammed CAR-T cells degranulate and retain cytokines such as IL2, IFNγ and TNF intracellularly in the co-cultures with Nalm6-CD19WT and not with Nalm6-CD19KO in a CD19 CAR-specific manner. To measure CAR-mediated proliferation of reprogrammed T cells using flow cytometry, total pool of cell trace violet (CTV) labelled PBMCs are treated with RNV-hCD19 then co-cultured with Naml6-CD19WT and Nalm6-CD19KO cell lines separately for 72 hrs. Only reprogrammed T cells proliferate preferentially in the cultures with Nalm6-CD19WT, not with Nalm6-CD19KO in a CAR-specific manner.

Reprogrammed T cells also show CD19 CAR-specific tumor cytotoxicity when co-cultured with Nalm6-CD19WT compared to co-culture with Nalm6-CD19KO as measured in short and long-term co-culture assays. Luminescence based method are used to measure short-term immediate CAR-specific cytotoxicity against Luciferase expressing-Naml6-CD19WT. Agilent’s xCELLigence assays are also used to measure short and long-term CAR-specific tumor cytotoxicity against Nalm6-CD19WT. xCELLigence assay performs continuous real-time cell analysis (RTCA) on any changes in numbers of Nalm6 tumor line using biosensors embedded in the culture plates. Addition of reprogrammed T cells at different E:T ratios demonstrate CD19 CAR-specific killing of Nalm6-CD19WT compared to Nalm6-CD19KO cells. Further, 72 hrs-long xCELLigence assay show CAR-specific killing and growth inhibition of Nalm6-CD19WT tumor cell line.

### Example 2

### HAL2 Packaging Cell Line Encoding Plasmid Constructions Expressing Engineered Versions of MLV Derived Gag/Pol and Env in a Parental HT-1080 Cell

The HAL2 packaging cell line was developed in similar fashion to the HA-LB packaging cell as described in Sheridan et al., MOLECULAR THERAPY Vol. 2, No. 3, September 2000. For this example, the HAII packaging cell line, also described in Sheridan et al. Mol. Ther. 2000, can be used. The HAL2 packaging cell plasmid constructs as well as the constructs for the HAII packaging cell line are described herein. The plasmids constructs were stably transfected into the parental HT-1080 cell line (ATCC-CCL-121). Alternatively, for those skilled in the art, the gag/pol and env MLV derived sequences can also be inserted and expressed using a lentiviral vector pseudotyped with VSG-g to stably transduce the MLV sequences in sequential transduction events to obtain a similar HAL2 packaging cell lines. After stably transfection or transduction of the gag/pol sequence, the gag/pol expressing HT-1080 intermediate is dilution cloned, with individual clones screened for high gag/pol p30 expression by Western blot analysis. The clones can also be screened for functional titer production by transducing the gag/pol clonal intermediate with a MLV vector that contains both 5′ and 3′ LTRs flanking a packaging signal, a selectable marker (e.g., neomycin resistance) or a marker gene (e.g., Emerald GFP) which also expresses a functional MLV env sequence, to confirm that the gag/pol packaging cell line intermediate is capable of producing high titer MLV viral particles. After confirmation of good titer vector production potential, the MLV env sequence is inserted into the selected gag/pol intermediate by stable transfection or stable transduction using a lenti-viral vector encoding the MLV env sequence. After the MLV sequences have been delivered, the packaging cell line expressing both gag/pol and env is dilution cloned and screened to identify the highest env expression as well as for confirmation of functional titer production using a similar MLV test vector capable of expressing a selectable marker or marker gene except without a viral env sequence to evaluate individual clone packaging cell line performance. The specific MLV gag/pol and env constructs used to create the HAL2 packaging cell line are further described below.

### MoMLV-Derived Gag/Pol Constructs

The HAL2 packaging cell line uses the original MoMLV-derived gag/pol plasmid pSCV10 (see, e.g., WIPO patent publications WO 91/06852 and WO 92/05266, the disclosures of each of which are incorporated herein by reference in their entirety) as used for the HA-LB MLV packaging cell line designed to reduce replication competent virus by homologous recombination events. The gag/pol construct in the HAII packaging cell line can also be used, which has reduced sequence homology to the retroviral vector and env expression constructs. In HAII, the gag/pol construct expression cassette pCI-WGPM contains degenerate code in approximately the first 400 nt of the coding region for gag, as well as deletions of all 5′ and 3′ untranslated sequences. In addition, the sequence coding for the last 28 amino acids of the pol gene is deleted, resulting in a truncated integrase gene. Plasmids pCI-GPM and pSCV10/5′,3′tr contain the same gag/pol cDNA as pCI-WGPM except that the 5′ area of gag contains the native sequence.

### MoMLV Derived Ampho Envelope Constructs

To reduce sequence overlap in the gag/pol and retroviral vector plasmids, the original 4070A-derived amphotropic expression plasmid pCMVenv^{am}Dra (Patent Application WO 91/06852) was used to generate two plasmids with either all 3′ untranslated sequences deleted after the env stop codon (pCMVenv^{am}DraLBGH) as used in the HA-LB packaging cell line, or all 3′ and 5′ untranslated sequences are deleted (pCMV-β/env^{am}) as used in the HAII packaging cell line.

### Creation of Vector Producing Cell Line (VPCL) From HAL2 to Produce MLV Viral Particles that Encode Emerald GFP as Well as miRNA Sequences for Down Regulation of Vector Expression in Off-Target or Non-Intended Specific Cell Types using the pBA-9B-emdGFPmir233-3p4TXv2 MLV Plasmid Vector

Plasmid construct pBA-9b-emdGFPmir233-3p4TXv2 (construct 7) has been modified for cell-specific detargeting. The basic pBA-9B-emdGFP sequence can be modified to also encode microRNA (miR) target sequences as an effective method of down regulating vector expression in cell type specific manner to increase the safety profile of the vector and prevent expression in non-intended cell types. FIG. 5 shows examples of miR target sequences used to impede expression in myeloid, B and NK cell types.

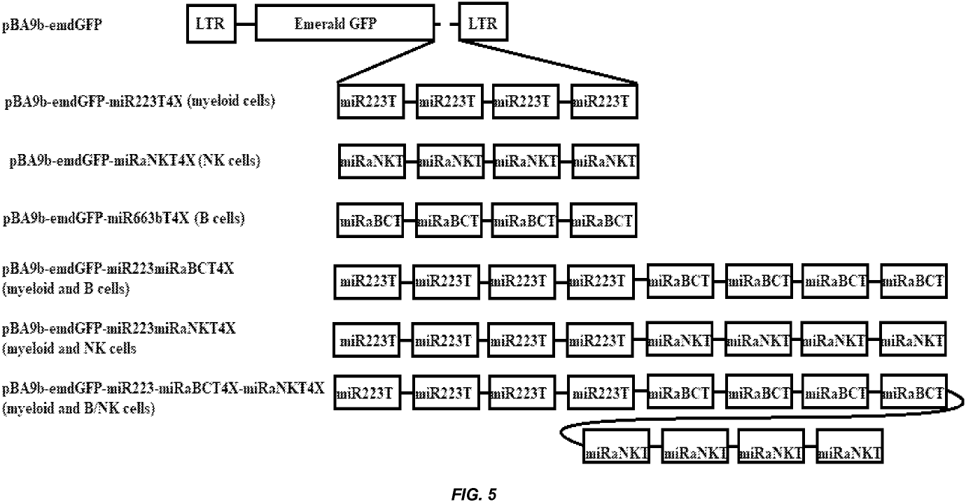

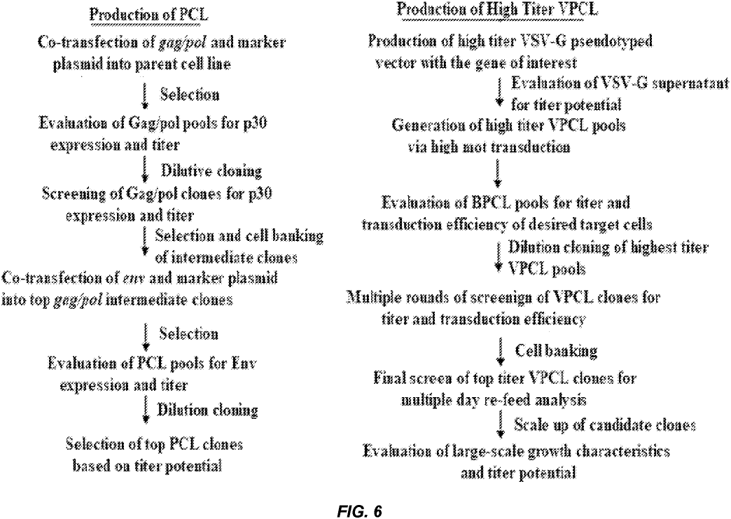

Retroviral non-clonal vector producing cell lines as well as subsequent production clones are established from the selected HAL2 clonal packaging cell line using a high multiplicity of transduction (“m.o.t.”) “high m.o.t.” approach with m.o.t.’s of >20 using a single or multiple back-to-back rounds of transductions using a VSV-G pseudotyped MLV vector particles. The multiplicity of transduction is defined as the number of infectious viral particles used per PCL cell for the production of VPCL non-clonal cell line. Typically, a PCL culture is seeded at 1 × 10⁵ cells/well in a 6-well plate one day prior to transduction. The appropriate volumes of vector supernatants are then added to PCLs (in the presence of 4, 5, 6, 7 or 8 µg/ml polybrene; corresponding to m.o.t.’s of 0.1, 0.5, 5, 25, and 125, respectively). After 20-24 h the vector supernatant is replaced with 2 ml of fresh media. To increase the m.o.t.’s, the transduction procedure can be repeated for a second day using the same volume of vector supernatant. Producer pools are grown to confluence and supernatants collected daily at 24, 48, and 72 h post-confluence to determine PCR transduction titers and show transfer of gene expression. Selected non-clonal pools are cloned using limited dilution seeding into 96 well plates that result in a single cell per well that are analyzed in several rounds of titer determination and transfer of expression assays as individual groups of clones expand. VPCL clones that have sustainable high titer production are cryopreserved to prepare frozen down a working stock that is tested for safety (sterility, mycoplasma, replication competent retrovirus as well as other viral adventitious agents as described in FDA points-to-consider and Guidance publications). Genomic viral sequencing of the viral particles that derive from individual clones is also performed to ensure accuracy of the genomic MLV sequences, as part of qualified cell bank stock characterization. Once qualified, a vial from the qualified cell bank stock can be further expanded and further tested under GMP to produce a GMP Master and Working cell bank stocks. The series of events for creating either Packaging Cell Line or Vector Producing Cell Line is summarized in FIG. 6.

### Example 3

### Viral Production for R&D Studies as Lab Scale Using Multiple Corning CellSTACKs® or Pilot Scale Perfusion Fed Cell Culture Systems for Adherent Cultures

The following example reviews cell culture viral production methods for growing adherent vector producer cell line (VPCL) cells to produce murine leukemia virus (MuLV) for small scale R&D Studies. Production MuLV virus based on the pBA-9b-emdGFPmir233-3p4TXv2 construct is used as an example, however this process can also be used for all MLV viral vectors described in this disclosure.

For this example, VPCL produced from the parental HT1080 cells (ATCC CCL-121) is described, however the method can also be used for VPCLs produced from HEK 293T, D17 or CF2 derived cells (CRL-1573, CCL-183, or CRL-1430, respectively), under the conditions of 37° C. under 5% CO₂ conditions. For long term storage, VPCLs are cryopreserved under liquid nitrogen conditions, stored in cryoprotectant plastic vials containing up to 1.0 × 10⁷ cells frozen in a cryoprotectant cell culture media solution containing 10% DMSO, and 50-90% fetal bovine serum in cell culture growth media solution. Upon thawing, cells are expanded by initially seeding into a T-75 flask with subsequent expansions into two T-175′s and then subsequently cultured into multiple T-175 flasks with the following growth medium:

| Complete DMEM Medium components: | | Ratio |
|---|---|---|
| 1. | DMEM High Glucose, w/o phenol red & w/o glutamine | 500 mL |
| 2. | FBS Defined gamma-irradiated | 25 mL |
| 3. | GlutaMax (Gibco) | 5 mL |
| 4. | Non-essential amino acids (100x NEAA Stock) | 5 mL |

After reaching confluence, cells are harvested with TrpZean® (Sigma) and neutralized with the same growth medium using standard cell culture methods. The cells are seeded into three 10-layer CellSTACKs® (Corning) at a seeding density of about 3.1 × 10⁴ viable cells/cm² in complete DMEM medium, to produce the virus. Each Cell Stack contained 1.1 L of the growth medium. The CellSTACKs® are incubated at 37° C. and 5% CO₂.

Approximately two days after seeding, the CellSTACKs® cultures will approach or reach confluence. After reaching confluence, the medium in each culture is replaced on a daily bases with fresh medium with spent cell culture media, containing produced virus, harvested after a fresh media exchange. Alternatively, fresh media feedings and vector harvesting can occur every 10-12 hours with same volume (1.1 L) of fresh growth medium. After a desired volume is collected, the harvest collections are then pooled for purification. Viral titers for the 3 harvests and the pool are listed in the following table.

| Harvest | Viral titer (# TU/mL) |
|---|---|
| Harvest #1 | 1.8 × 10^6 |
| Harvest #2 | 2.4 × 10^6 |
| Harvest #3 | 2.4 × 10^6 |
| Pool of 3 harvests | 2.1 × 10^6 |

### Large-Scale Vector Production of Adherent VPCLs Using a Corning CellCube® Perfusion System

If larger amounts of vector are required, large-scale retroviral vector production using adherent cell culture expansion trains can be performed using multiple single use systems such as fibrous disks, micro-carrier beads or fixed bed like systems like the iCellis® system (Pall Corporation, NY). To seed a CellCube® system, expansion of the VPCLs are performed in 225-cm² tissue culture flasks using DMEM formulated with 10% γ-irradiated defined fetal bovine serum. Cells are allowed to expand for about 3-4 days until sub-confluence. The cells are then progressively passaged while increasing the surface area to 4 × 10-layer Cell Factories (Nalge Nunc International, IL) to reach the required number of cells to inoculate the CellCube® system. Fresh media perfusion feeding is controlled based on glucose consumption with up to a maximum media exchange of four system volumes per day. Production volume depending on the number of Cell Cube modules used from 200 to 1000 liters over a period of up to about 13-16 days. Representative bioreactor samples are taken for metabolic profile and PCR transduction titer analysis.

### Example 4

### Adaptation of HT-1080 MuLV Viral Vector Producer Cell Line from Serum and Adherence Dependence to a Serum Free Suspension Culture

Briefly, a serum free adaptation process is performed after screening and identification of a suitable dilution clone of HT-1080 vector producing cell line. The adaptation process is initiated by seeding approximately 2×10⁷ cells into a 125 mL shaker flask containing 10 mL of 5% serum containing conditioned media and 10 mL of a selected serum free media of choice, resulting into a reduced serum concentration of 2.5%. In this example, the serum free media is FreeStyle 293 Expression Media distributed through Invitrogen Corp, Carlsbad, CA, however for those skilled in the art, a comparable or custom serum-free media can also be used. The culture is placed on a shaking platform located in a tissue culture incubator with both temperature and CO₂ gas control. The shaking platform is set to about 80 RPM and the incubator is set to a 37° C. and a preferred 5% CO₂ conditions. Every 3-7 days, the culture is re-fed by collecting cells that are in suspension and reseeded into a new shaker flask containing 10 mL of the same initial conditioned media and 10 mL of fresh serum free media maintaining a level of serum of approximately 2.5%. The culture is examined at each re-feeding event with viable cell counts performed as needed to check for cell propagation. When the cells show evidence of growth based on cell doubling or glucose consumption, a serum concentration of 1.67% is then targeted by adjusting the volume amount of condition media and fresh serum free media. The culture again is examined and refed every 3- 7 days. When the cells show evidence of growth, a serum concentration of 1.25% is targeted by again adjusting the volume of conditioned media and fresh serum free media. This process is continued targeting subsequent serum conditions of 1.0%, 0.9%, 0.83% serum conditions until the cells are in 100% serum free conditions. During this adaptation process the cell culture is expanded to approximately 200 mL volume in a 1,000 mL shaking flask targeting a minimal viable culture of approximately 0.5 to 1.0 ×10⁶ cells/mL. Once the cells reach 100% serum free conditions, the cells are continuously passaged under serum free conditions isolating single suspended cells by allowing heavier clumping cells to settle for short periods of time without agitation. Once the culture consists of approximately 95% population of the single cell suspension consistently, the culture can be frozen in cryopreservation media consisting of 10% DMSO and 90% serum free media using standard mammalian cell freezing conditions.

Materials Required:
FreeStyle 293 Expression Media, Invitrogen Corp., Carlsbad, CA
125-mL and 1000-mL vented shake -flasks
DMSO, USP (Cryoserv, Bionche Pharma USA, Lake Forest, IL

### Example 5

### Adaptation of Producer Cell Line to Suspension Culture and Producing Virus in a Rocking Bag Bioreactor System For Pilot/Pre-Clinical Scale Production

Using the previously described process for adapting a VPCL adherent clone to a serum free suspension VPCL, cell expansion is initiated using a series of shaker flasks containing the indicated amount of cell culture media per flask: 125-mL (20 mL culture); 250-mL (40 mL); 500-mL (100 mL); and 1-L (200 mL) all from Corning. Cell expansion is performed using the fully defined serum-free medium (FreeStyle 293 Expression Media, Gibco Cat# 12338), supplemented with 0.1% human serum albumin (HSA, from Octapharma USA, NJ). The cultures are incubated at 37° C. and 5% CO₂ with shaker speed of about 80 rpm. Five 1-L shake flask cultures are used to inoculate a WAVE bioreactor (WAVE 20/50 EHT, Cytiva Healthcare Life Sciences/GE Healthcare, MA) containing a 20-L Cellbag with 10 L working volume. The initial cell density in the bioreactor is about 4 × 10⁵ viable cells/mL (viability 91%) at 37° C. The initial operating conditions are: 5% CO₂, rocker speed 15 rpm, angle 6°, air flow rate 0.2 L/min. The pH control is set at about 7.2, and DO control at about 40%. Both pH and DO controls are implemented by a WAVE POD console system.

After the cell density reaches ~1 × 10⁶ viable cells/mL, a cell perfusion process is started using a hollow fiber (Part# CFP-6D-6A) cartridge (Cytiva Healthcare Life Sciences/GE Healthcare, MA). The feed (and permeate) rate is initially set at -0.25 volume/day, and progressively increased with cell density for up to an ~3.8 volumes/day. A total of 180 L of permeate containing the virus is harvested within a 15 day period. Approximately 300 L of harvested material can be collected using the 50 L WAVE bags using a working volume of 25 L. The viral titer in the 180 L - 300 L harvests can be 5-8 × 10⁶ TU/mL.

### Large-Scale Vector Production of Suspension Adapted VPCLs Using a Single Use Bioreactor (SUB) Perfusion System

As a SUB bioreactor scale-up example, a 100 L SUB (Thermo Fisher Scientific) system with a minimal 52 L working volume is used to produce approximately 900-1000 L of clarified vector. Cells are expanded as previously described for inoculation of a WAVE bioreactor rocking system. After SUB inoculation, similar to the WAVE, the cells are grown to higher cell densities by using a perfusion process with continuous fresh medium feeding and clarified permeate harvest collection (over period of 14 to 21 days) in a Single Use Mixer (SUM) chilled to 2-8° C.

Perfusion feeding of the culture starts approximately 3 days after the bioreactor is seeded. The cell culture medium is Gibco FreeStyle 293 Expression Medium (Thermo Fisher Scientific) supplemented prior to use with 0.1% human serum albumin (HSA) with the addition of a low level concentration of an antifoam agent based on polydimethylsiloxane such as Anti-Foam B (Sigma-Aldrich).

During the perfusion process in the SUB bioreactor system, suspension cells are retained in cell culture circulation, while the cell culture supernatant containing the vector product is harvested following tangential flow microfiltration through a hollow-fiber cartridge with a 0.45 µm nominal pore. The “permeate”, or clarified vector harvest, is collected into a single-use harvest bag within a jacketed single-use mixer (SUM), which is controlled to 2-8° C. Harvest collection starts between days 5-9, when cell density reaches 3-5 × 10⁶ viable cells/mL. The collection process lasts for 11 to 17 days. The production run is ended when approximately 900-1000 L of clarified vector harvest is obtained, which takes approximately 14 to 21 days from the time of seeding the bioreactor.

Throughout the producer cell line expansion and vector production process, the temperature of the bioreactor is controlled at 37° C. pH is controlled at 7.20±0.15 by the controller-automated addition of sodium bicarbonate, and dissolved oxygen is controlled at 40% saturation set point by sparging of O₂ into the bioreactor. The CO₂ gas flow rate is set at 5% of that of the air flow rate. Glucose level is on-line monitored as an indicator for feed rate increases. When the density of cells in the bioreactor reaches approximately 2.5 × 10⁶ viable cells/mL, or the level of glucose in bioreactor decreases to 0.5 g/L (from an initial 5 g/L), perfusion of fresh medium is initiated. As cell density increases over time, the feed rate increases. Feed rate increase is automated via an Applikon controller utilizing a user specific and developed automation code based on BioXpert W7 software (Applikon Biotechnology). Fresh medium is fed to the bioreactor to supplement glucose and nutrient depletions due to increased consumption of nutrients in the bioreactor as cell density increases. The rate of clarified vector harvest withdrawn from the tangential-flow hollow-fiber cartridge is increased as the medium feed rate increases. Both the feed and permeate rate increases are automated based on on-line glucose measurement from the bioreactor. The weight (volume) of the bioreactor is also controlled using the system by adjusting the permeate rate (volume withdrawn from the bioreactor), after its concurrent increase with the feed rate, by the same user specific automation code.

### Example 6

### Viral Purification and Concentration.

Purified viral preparations are not only used as pharmaceutical preparations for clinical use but also required for (1) coating of ELISA plates as a purified capture antigen to detect antiviral antibodies, and (2) used as a purified immunogenic antigen in animals to generate positive control anti-viral antibodies for ELISA assays or other immunogenic viral detection methods. Virus of the disclosure are manufactured under various modalities by either transient transfection either 293T, a 293 cell line expressing MLV gag-pol (e.g., see Burns et al. PNAS 90:8033-8037 1993) or HT1080 cells, or from a vector producing non-clonal cell line pool, or from a cloned vector producer cell line. Depending on cell line adaptation, the medium can be with serum or serum free, and the cells can be grown as adherent cells or in suspension, with cell culture supernatant collected in batch harvests or collected under perfusion mode and stored under refrigerated 2-8° C. conditions. The culture supernatant is harvested, and stored for up to 2 weeks at 4° C. This bulk harvest is filtered through a dead end 0.45 micron filter cartridge or through using tangential flow microfiltration using hollow fiber cartridges or plate and frame filtration systems used to remove large cellular debris. After filtration, the vector is treated with 2-5 Units/mL Benzonase® (EMD Millipore, Damstadt Germany) in the presence of 2 mM MgCl₂ incubated overnight in the range of 15-30 hours at a minimum temperature of 4° C. to digest cellular DNA (Shastry et al., Hum Gene Ther., 15:221, 2004) and then subjected to a concentration step.

The concentration step can be performed using tangential flow ultra-filtration or by anion ex-change chromatography. For ultrafiltration, concentration of viral vector material is achieved using a 500 MW cut-off membrane designed to retain the large viral particles within the recirculating loop. After sanitization and neutralization of the system, post treated vector material is concentrated in a re-circulation loop that starts in the concentrating vessel, through a peristaltic pump, through a 500 MW hollow-fiber cartridge, and then returned to the concentrating vessel. Once all air has been removed from the recirculation system, the permeate outlet is open to begin the concentration process. The flow of permeate is set to approximately ⅒ the flow rate of the recirculation rate. The recirculation rate is dependent on the size of the hollow fiber cartridge but typically it is set to approximately 75% of the maximum speed at which the pump speed and shear rate is not damaging to the virus. As permeate waste is collected, additional vector material is added to the loop until the material reaches a desired concentrated range normally a 10 to 50x fold increase in titer and a corresponding reduction in volume. Once the target concentration is achieved, formulation buffer is used to diafilter and buffer exchange the vector into the desired pH neutral, isotonic Tris-buffered sucrose solution. This material can then be filter sterilized and used as is or can be subject to further chromatography purification. Polishing chromatography can be used by either using (1) a multimodal resin such as Capto Core 400 (Cytiva/GE Healthcare) which can further remove small molecular weight charged proteins or (2) the use of standard size exclusion chromatography, such as S-500 resin (Cytiva/GE Healthcare), which serves as a buffer exchange as well as removes small molecular weight protein. After chromatography, the vector can be formulated with required excipients to provide stability, 0.2 µ filter sterilized, vialed and frozen at minus 65° C. or below.

As an alternative to ultrafiltration, vector concentration can also be achieved using AEX chromatography (see, e.g., U.S. Pat. No. 5,792,643; Rodriguez et al., J Gene Med., 9:233, 2007; Sheridan et al., Mol. Ther., 2:262-275, 2000). The benzonase treated viral preparation is loaded on an anion exchange column and the virus is eluted in a stepwise NaCl gradient. The fraction containing the virus can be identified by PCR assay, or by A215, A280 as well as A400. Positive fractions are collected and pooled. The pooled preparation is subsequently loaded on a size exclusion column (SEC) to remove salt as well as other remaining small molecular weight contaminants as well as condition the virus into SEC formulation buffer (20 mM Tris based isotonic solution consisting of 90 mM NaCl; 1% sucrose, 1% mannitol adjusted to pH 7.2 with HCl). The SEC is run under isocratic conditions with formulation buffer and the viral fraction from the SEC column is collected from the void volume. Positively identified fractions are pooled and supplemented to a final 1 mg/mL human serum albumin, filtered through a formulation pre-wetted sterile 0.2 µm filter, aliquoted and frozen at minus 65° C. or below. All components used for the process are USP compendial grade materials with the manufacturing process, which can be performed under GMP requirements, for clinical use. The viral preparation is released based on standard testing such as sterility, mycoplasma and endotoxins, with purity and consistency evaluated by SDS PAGE gel analysis. Titer is determined as Transducing Units (TU) by PCR quantitation of integrated viral DNA in target cells. The final product is targeted to have a titer range of 1×10⁸ to 1×10⁹ TU/ml.

### Example 7

### Construction and Characterization of Measles H-CD8scFv and Truncated Measles F Protein Molecules

In another embodiment, MLV-measles hybrid vectors are provided to target CD8 positive T-cells, Measles hemagglutin “H” constructs are modified to encode a chimeric sequence of a single chain antibody specific for CD8 expressing cells. In the design, the H protein cytoplasmic tail is used to anchor the chimeric protein to the virus with the receptor binding sequences, are either mutated or deleted to destroy H receptor binding. As demonstrated in the expression construct HstalkscFvhl, receptor binding is rather achieved using the fused CD8 targeting scFv sequences through a linker sequence. As demonstrated in the expression construct HstalkscFvlh improvements or alternative targeting is achieved by identifying the orientation of alternative heavy and light sequences of this and other receptor specific scFv binding sequences. In the design, the Edmonston B strain measles sequence is used but other strains of measles virus can be used to donate F and H proteins for equivalent modification. To achieve viral fusion upon CD8 cell binding, a separate chimeric MLV env construct, without the Pit2 binding recognition sequences, is fused to the measles “F” fusion protein as described for construct EdB fusion P DNA-4070A tail. This chimeric construct is bound to the viral particle using the cytoplasmic tail of the amphotropic envelope. In an alternative design, a tail-less F protein can also be used to achieve viral particle fusion.

### Example 8

### Construction and Testing of PCL/VPCL with Targeted CD 8 Envelope Using a MoMLV Hybrid Envelope with Measles “F” Fusion Protein and “H” Binding Proteins Modified for CD8 Positive T-Cell Targeting

To create a packaging cell line capable of producing MoMLV viral particles capable of targeting CD8 positive T-cells, the measles H-CD8scFv and truncated Measles F protein constructs described in Example 7 are used in place of either the pCMVenv^{am}DraLBGH or pCMV-β/env^{am} envelope constructs described in Example 2 to create a packaging cell line. Briefly, the plasmids constructs encoding H-CD8scFv (construct 52, pCMVenv^{MFhlCD8}DraLBGH) and the truncated Measles F protein expression vectors (construct 53, pCMVenv^{MtF}DraLBGH), are transfected sequentially into the characterized gag/pol packaging cell line intermediate by stable plasmid transfection. As previously indicated, those skilled in the art can also use engineered lentiviral vectors to stably transduce the gag/pol intermediate cell line with the targeting H-CD8scFV and truncated measles F (mF) sequences. In one embodiment, the measles F protein vector is initially delivered to create a gag/pol intermediate cell line that also expresses the measles F protein. This allows for an alternative gag/pol-mF intermediate cell line that only requires the addition of viral hybrid envelope targeting sequences to create a packaging cell line with alternative viral targeting potential. This new gag/pol-F protein intermediate is serially diluted to produce dilution clones, with clones screened for the highest co-expression of both the gag/pol and mF protein sequences by Western blot analysis. Once several clones have been identified for stable expression, the clones can be functionally tested by introducing into the cell line a similar MLV test vector capable of expressing a selectable marker as well as a viral targeting H-CD8scFV sequence to evaluate viral packaging cell line performance. In performing the titer analysis, naive test cells to test transfer of expression will need to be CD8 positive cells such as TALL-104 (ATCC CRL-11386); Molt4 (ATCC CRL-1582) or on adherent cells modified to stably express the CD8 receptor such as with PC-3 (ATCC CRL-1435) or HT0180 (ATCC CCL-121). Once the clonal gag/pol-mF cell line intermediate has been identified, the cells can then be stably transfected or transduced with the targeting H-CD8scFV expression vector. The targeting H-CD8scFV packaging cell line is subsequently serially diluted, with individual clones screened for all relevant viral protein sequences: gag/pol, mF, and H-CD8scFV. Clones with the highest expression are again tested for functional performance by introducing a MLV test vector expressing a fluorescent marker and/ or drug selectable resistance marker to evaluate stable titer production by transfer of expression of viral particles onto naive CD8 positive titering cells.

### Creation of Vector Producing Cell Line (VPCL) Using the CD8 Targeting MLV Packaging Cell Line

To produce a VPCL using the CD8 targeting MLV packaging cell line described above, the identical MLV vector pBA-9b-emdGFPmir233-3p4TXv2 as demonstrated in Example 2, or an alternative MLV vector construct is used to stably transfect the CD8 targeting PCL. Alternatively, the MLV vector construct is used to produce transiently produced VSV-g or ampho-derived vector particles to stably transduce the CD8 targeting MLV packaging cell line. As previously described above, retroviral non-clonal vector producing cell lines, as well as subsequent production clones, are established according to the outline referenced in FIG. 6. However, for this example, the CD8 targeting PCL is used in lieu of the HAL2 clonal packaging cell line using the same high multiplicity of transduction “high m.o.t.” approach with m.o.t.’s of >20 used in a single or multiple back-to-back rounds of transductions using VSV-G pseudotyped MLV produced from a gag/pol intermediate cell line or using amphotropic vector generated particles by transiently transfecting into HAL2 cells with the MLV vector. Typically, a PCL culture is seeded at 1 × 10⁵ cells/well in a 6-well plate one day prior to transient transduction. The appropriate volumes of vector supernatants are then added to PCLs (in the presence of 4-8 µg/ml polybrene) corresponding to m.o.t.’s of 0.1, 0.5, 5, 25, and 125. After 20-24 h the vector supernatant is replaced with 2 ml of fresh media. To increase the m.o.t.’s, the transduction procedure can be repeated for a second day using the same volume of vector supernatant. Producer pools are grown to confluence and supernatants collected daily using a daily media refeed schedule at 24, 48, and 72 h post-confluence to determine PCR transduction titers and/or assess transfer of gene expression as described above using a CD8 positive cell line as the titering cell line. Selected non-clonal pools are cloned using limited dilution seeding into 96 well plates that result in a single cell per well that are analyzed using several rounds of titer determination and transfer of expression assays as individual clones expand. VPCL clones that have sustainable high titer production are cryopreserved to prepare frozen down working stocks that is tested for safety (sterility, mycoplasma, replication competent retrovirus as well as other viral adventitious agents as described in points to consider FDA publications). Genomic viral sequencing of the viral particles should also be performed that derive from individual clones to ensure accuracy of the genomic MLV sequences, as part of a qualified cell bank characterization. Once qualified, a vial from the qualified cell bank stock can be further expanded and further tested under GMP to produce a GMP Master and Working cell bank stocks for eventual clinical production.

### Example 9

### Construction and Testing of PCL/VPCL with Targeted CD 4 Envelope Using a MoMLV Hybrid Envelop with Measles “F” Fusion Protein and “H” Binding Proteins for CD4 Positive T-Cell Targeting

To create a packaging cell line capable of producing MoMLV viral particles capable of targeting CD4 positive T-cells, the measles H-CD4scFv and truncated Measles F protein constructs described in Example 7 are used in place of either the pCMVenv^{am}DraLBGH or pCMV-β/env^{am} envelope constructs described in Example 2 to create the packaging cell line. Briefly, the plasmids constructs H-CD4scFv and the truncated Measles F protein expression vectors are transfected sequentially into the characterized gag/pol packaging cell line intermediate by stable plasmid transfection. As previously indicated, those skilled in the art can also use engineered lentiviral vectors to stably transduce the gag/pol intermediate cell line with the targeting H-CD4scFV and truncated measles F (mF) sequences. In one embodiment, the measles F protein vector is initially delivered to create an alternative gag/pol intermediate that also expresses the measles F protein. This allows for a gag/pol-mF intermediate cell line that only requires the addition of viral targeting sequences to create a packaging cell line with alternative viral targeting potential. This gag/pol-F protein cell line intermediate is serially diluted to produce dilution clones, with clones screened for the highest co-expression of both the gag/pol and mF protein sequences by Western blot analysis. Once several clones have been identified for stable expression, the clones can be functionally tested by introducing into the cell line a similar MLV test vector capable of expressing a selectable marker as well as a viral targeting H-CD4scFV sequence to evaluate viral packaging cell line performance. In performing the titer analysis, naive test cells to test transfer of expression will need to be CD4 positive cells such as CCRF-CEM (ATCC CCL-119); A301 or on adherent cells modified to stably express the CD4 receptor such as with PC-3 (ATCC CRL-1435) or HT0180 (ATCC CCL-121). Once the clonal gag/pol-mF intermediate has been identified, the cells can then be transfected or transduced with the targeting H-CD4scFV expression vector. The targeting H-CD4scFV packaging cell line is subsequently serially diluted, with individual clones screened for all relevant viral protein sequences: gag/pol, mF, and H-CD4scFV. Clones with the highest expression of all proteins are again tested for functional performance by introducing an MLV test vector expressing a fluorescent marker and/or drug selectable sequence to evaluate stable titer production by transfer of expression of viral particles on naïve CD4 positive test cells.

### Creation of Vector Producing Cell Line (VPCL) Using the CD4 Targeting MLV Packaging Cell Line

To produce a VPCL using the CD4 targeting MLV packaging cell line described above, the identical MLV vector pBA-9b-emdGFPmir233-3p4TXv2 as demonstrated in Example 2 or an alternative MLV vector construct is used to directly stably transfect the CD4 targeting PCL. Alternatively, and in another embodiment, the MLV vector construct is used to produce transiently produced VSV-g or ampho derived vector particles to stably transduce the CD4 targeting MLV packaging cell line. As previously described above, retroviral non-clonal vector producing cell lines, as well as subsequent production clones, are established according to the outline referenced in FIG. 6. However, for this example, the CD8 targeting PCL is used in lieu of the HAL2 clonal packaging cell line using the same high multiplicity of transduction “high m.o.t.” approach with m.o.t.’s of >20 used in a single or multiple back-to-back rounds of transductions using VSV-G pseudotyped MLV produced from a gag/pol intermediate cell line or using amphotropic vector generated particles by transiently transfecting into HAL2 cells with the MLV vector. Typically, a PCL culture is seeded at 1 × 10⁵ cells/well in a 6-well plate one day prior to transient transduction. The appropriate volumes of vector supernatants are then added to PCLs (in the presence of 4-8 µg/ml polybrene) corresponding to m.o.t.’s of 0.1, 0.5, 5, 25, and 125. After 20-24 h the vector supernatant is replaced with 2 ml of fresh media. To increase the m.o.t.’s, the transduction procedure can be repeated for a second day using the same volume of vector supernatant. Producer pools are grown to confluence and supernatants are collected daily using a daily fresh media refeed schedule at 24, 48, and 72 h post-confluence to determine PCR transduction titers and/or assess transfer of gene expression as described above using a CD4 positive cell line. Selected non-clonal pools are cloned using limited dilution seeding into 96 well plates that result in a single cell per well that are analyzed using several rounds of titer determination and transfer of expression assays as individual clones expand. VPCL clones that have sustainable high titer production are cryopreserved to prepare frozen down working stocks that is tested for safety (sterility, mycoplasma, replication competent retrovirus as well as other viral adventitious agents as described in points to consider FDA publications). Genomic viral sequencing of the viral particles that derive from individual clones is also performed to ensure accuracy of the genomic MLV sequences, as part of qualified cell bank stock characterization. Once qualified, a vial from the qualified cell bank stock can be further expanded and further tested under GMP to produce a GMP Master and Working cell bank stocks for eventual clinical production.

### Example 10

### In Vivo Testing of Detargeting of B Lymphoma Cells with miRNA to Prevent Vector Expression (GFP) in Tumor Cells

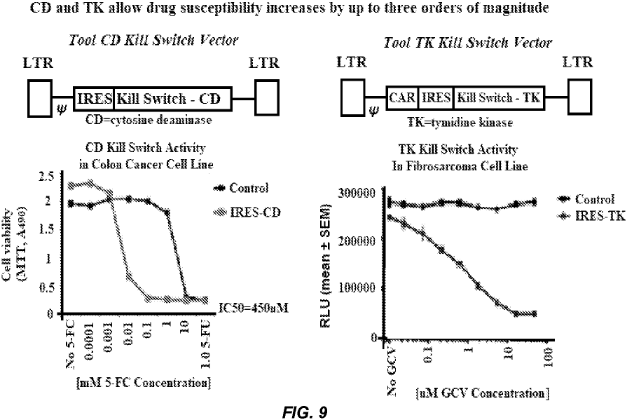

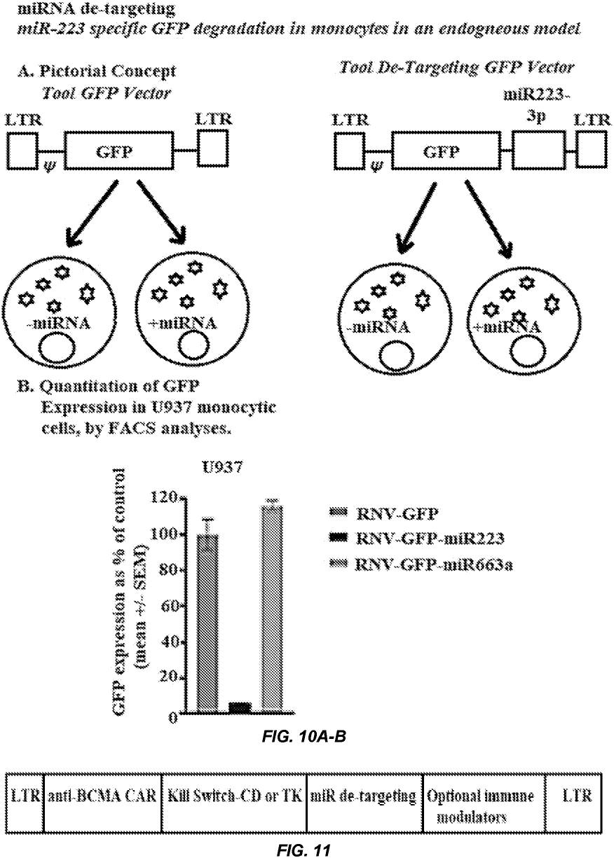

Differentiated cell types express unique sets of miRNAs, small noncoding RNA that act as posttranscriptional regulators of gene expression by degrading their target mRNAs. Addition of a specific miRNA sequences to the transcriptional cargo of a non-replicating retrovirus leads to cell type-specific degradation of the retrovirus such that the retrovirus can be effectively turned off in cells in which retroviral transduction and expression is undesirable. To demonstrate the efficacy of this approach, HT1080 human fibrosarcoma cells were engineered to overexpress the miRNA R223-3p, which it does not normally express, using a lentviral vector. These cells were then challenged with GFP vectors made from plasmids pBA-9b-GFP (c45) and pBA-9B-GFPmiR 223-3p4TX (c7) with results. There was an almost complete shutdown of GFP expression from the GFP-miRT223-3p vector in HT1080cells expressing miR223-3p but equivalent good levels of GFP expression using unmodified GFP in cells with or without miR223-3p expression. To show this effect in a less engineered situation, the monocyte cell line U937 was shown to express GFP from unmodified vector or from a vector with an irrelevant miRNA target (663A-T) and not from vector with miR223-3p (FIG. 10). These experiments show the capability to shut down gene expression using miRT modified vectors in a specific cell type expressing the matching microRNA. This capability was also demonstrated for vectors encoding an IRES CD Module, showing that the configuration for the kill-switch gene (IRES-Kill-Switch, this case cytosine deaminase) is equally amenable to this type of expression control. Together, these data confirm that miRNA sequences expressed by a transduced cell can be leveraged to extinguish expression of the RNV payload.

### Example 11

### In Vivo Therapeutic Effect of RNV Encoding an Anti-Mouse CD19 CAR by Direct Administration in a Syngeneic Mouse Model of Lymphoma

The plasmid pBA-9b-mCD19(1D3)-IRESyCD (c40) encodes an anti-mouse CD19(ID3) CAR vector described in WO2020/142780 with an IRES-CD cassette, and is used to make infectious vector as described in Examples 2-6.

The A20 lymphoma line (luciferized) in Balb/c mice (Kueberuwa et al., J. Vis. Exp., (140), e58492, 2018) was used a model to evaluate the efficacy of in vivo infection of the mouse antiCD19 CAR RNV construct, (mCD19-RNVCAR, mCD19 (1D3)-IRES yCD(v)), a mouse anti-CD19 scFv 1D3, followed by the murine CDS transmembrane domain, followed by murine 4-lBB intracellular domain, followed by murine CD3i (construct 4) intracellular domain. Briefly, one day prior to implantation of A20 lymphoma cells, 6 to 8-week old BALB/c mice were administered 150 mg/kg cyclophosphamide into the peritoneum (IP). Cyclophosphamide pretreatment allows A20 robust tumor engraftment. A20 B-cell lymphoma cells were injected IV (1E5 cells in 200 µL) on day 0. The vector, mCD19 (1D3)-IRES yCD(v), was injected at a dose of 1E8 TU per day for four consecutive days, starting at day 3 after A20 implantation.

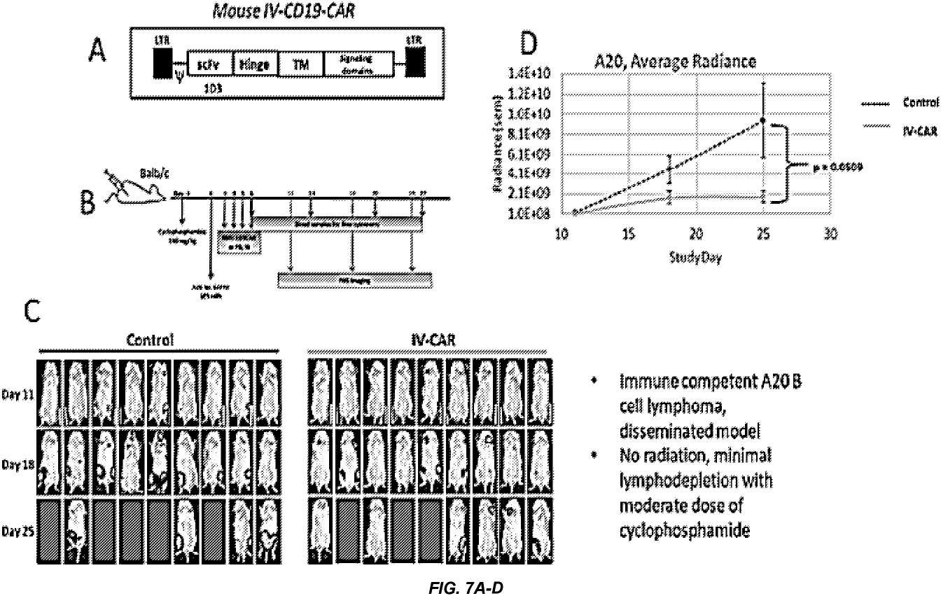

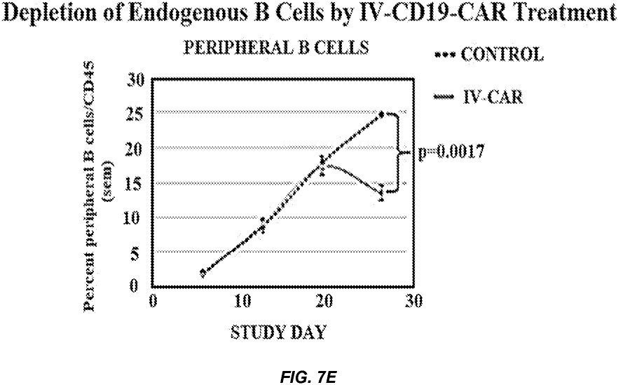

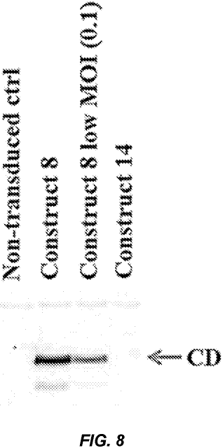

mCD19 (1D3)-IRES yCD(v) treatment led to lower A20 tumor burden compared to vehicle treated controls by day 25 as assessed by luminescent signal from A20 lymphoma cells (see FIG. 7). The significant decrease in numeric radiance is also visually apparent (see FIG. 7C, and there was an apparent survival advantage for the treatment group of 6/10 with two animals with no detectable tumor survivors compared to 4/10, no animals with no detectable tumor for the control group). The decrease in A20 tumor burden shows that IV administration of mCD19-RNVCAR vector inhibits A20 tumor growth. For tumor growth inhibition to occur after IV administration of mCD19 (1D3)-IRES yCD(v), the vector must enter the circulating T cells, the mCD19-CAR must get expressed on the surface of those T cells, those T cells must then home to sites of the disseminated tumor, and finally, the mCD19-CAR on the surface of the T cells must engage CD19 on the A20 tumor cells and activate killing of the A20 tumor cell.

### Example 12

### De-Activation of CAR-T Cells by Use of Prodrug Activating Genes

### A. In Vitro Killing of Infected Cells With the CD Gene Plus 5-FC and the TK Gene Plus Ganciclovir - See FIG. 9 Plus the Legend

### B. In Vitro Deactivation of CAR-T Cells Using the Yeast Cytosine Deaminase (CD) Kill Switch, Which Converts the Prodrug Flucytosine to 5-FU, Mitigates Acute Cytokine Release Syndrome (CRS) and Relieves Long-Term B Cell Deletion in Vitro

The in vitro and in vivo assays to measure Cytosine Deaminase activity and ability to kill cells is described in U.S. Pat. Publ. No: US20140178340A1 and US9732326B2. In this embodiment, the CD is used as a kill switch to ablate RNV-transduced CD19CAR positive cells thereby ending therapeutic treatment of the CD19 CAR activity. The acute, CRS, and long-term, B cell aplasia, can both be mitigated by deploying the yeast CD kill switch built into a CD19 CAR vector e.g. constructs 8 and 11 Expression and activity of polypeptides having cytosine deaminase engineered to be expressed in CD19CAR RNV can be confirmed through in vitro assays described in U.S. Pat. No. 9,732,326B2 and 5-FC kill curves compared for cells transduced with yeast CD+ construct 8) and CD-vectors (construct14). These data show an almost 100 fold increased sensitivity to 5FC for CD+ cells compared to CD- cells.

In another embodiment, the polypeptide having cytosine deaminase is replaced with optimized thymidine kinase (TKO) as the kill switch. The sequence of TKO, in vitro and in vivo assays to measure thymidine kinase activity and ability to kill cells is described in U.S. Pat. Publ. No. US20150273029A1. Similar to cytosine deaminase, when added to RNV containing additional therapeutic genes, use of TKO allows activation of common anti-herpetic drugs such as ganciclovir, acyclovir, valacyclovir (Valtrex™) or other analogues by phosphorylation in situ leading to cell killing of RNV-transduced and neighboring cells. Acute CRS, and long-term B cell aplasia, can therefore both be mitigated by deploying the TKO kill switch built into a CD19 CAR vector when combined with anti-herpetic drugs.

The acute side effect of CD19CAR treatment in humans is associated with higher levels of certain cytokines produced by CAR-T function, cytokine release syndrome (CRS). This side effect can lead to extended hospitalization and can be life threatening. The ability to decrease the cytokine response mediated by CAR-T cell function by reduction in the number of CAR-T cells without eliminating the response entirely can increase the safety and reduce side effects associated with traditional CAR-T. To evaluate the ability of the kill switch to reduce CRS, CD34+ transplanted mice are treated IV with CD19CAR(v) or CD19CAR-yCD(v) and two days later administered various concentrations of 5-flucytosine between 50 and 500 mg/kg. Without flucytosine, approximately 30% of mice develop CRS-like symptoms and show elevated blood cytokine concentration of IL-6, IFN-g, GM-CSF, and TNFa. Those mice showing elevated cytokine levels also show strong CD19CAR persistence in the spleen and bone marrow by PCR and flow cytometry. At a high dose of flucytosine (500 mg/kg), none of the mice treated with CD19CAR-yCD(v) show elevated peripheral blood cytokines or CD19CAR persistence in spleen and bone marrow, while the percent of mice experiencing CRS-like symptoms remains unchanged in mice treated with CD19CAR(v). This demonstrates that cytokine elevation and CRS-like symptoms are associated with the presence of the CD19CAR. However, at low doses of flucytosine (50 - 150 mg/kg), none of the mice treated with CD19CAR-yCD(v) show elevated cytokines or CRS-like symptoms while the CRS affect in CD19CAR(v) remains at 30%. In animals treated with CD19CAR-yCD(v), there is still detectible CD19CAR and persistent B cell aplasia suggesting that low dose of the prodrug can decrease cytokine levels associated with CAR function without eliminating the CAR and CAR function. The ability to tune or modulate the cytokine response with low dose prodrug opens the possibility of improving safety without disabling function of the CD19CAR-yCD(v)-transduced T cells.

### C. In Vivo Deactivation of CAR-T Tells Using the TKO Kill Switch, Mitigates Long-Term B Cell Aplasia Effects and Acute Cytokine Release Syndrome (CRS)

In another embodiment, yeast cytosine deaminase (yCD) is replaced with TKO in the CD19CAR-expressing RNV. Long-term, B cell aplasia effects, and acute, CRS, effects associated with CAR-T activity can both be mitigated by deploying the TKO kill switch built into a CD19 CAR vector. Similar in vivo studies are run as described as above with similar results: activation of the TKO kill switch with a low concentration of prodrug (e.g. 5 - 20 mg/kg ganciclovir IP, twice per day) reduces peripheral cytokines associated with CRS while allowing for the persistence of CD19CAR-transduced T cells to kill tumor cells; and, activation of the TKO kill switch with a high dose of prodrug (e.g. 50 mg/kg ganciclovir IP, twice per day) effectively eliminates the CAR transduced cells and the activity of the CAR-transduced cells, namely B cell depletion.

### Example 13A

### In Vitro Testing of IL-12p70 Expressed in a Non-Replicating Retroviral Vector Also Expressing a Chimeric Antigen Receptor Targeted Against CD19

The addition of human IL-12p70 as a single chain construct (construct 16; SEQ ID NO:3) to a human CAR-expressing construct supports sustained and potent T cell activity of the CAR and promotes anti-cancer immune activity of non-CAR transduced immune cells. Primary peripheral T cells or T cell lines (e.g. TALL-104; Jurkat) are transduced with RNV encoding human CD19-targeted CAR or encoding CD19-targeted CAR that co-expresses IL-12p70 transgene (pBA9b-CD19CAR, construct 6 or 8, and pBAb-CD19CAR-IL-12p70, construct 16) at varying MOIs (e.g., 0.1, 1, and 10). Western blot on samples of the transduced cells is run and confirms a dose-dependent expression of CD19 CAR and for construct 16, increasing IL-12p70 protein levels corresponding to the increasing MOIs used. The RNV-transduced cells are labeled with a proliferation monitoring dye (e.g., cell trace violet). The cells are then mixed with CD19-expressing cells (e.g., NALM-6 +/- CD19 expression; primary B cells, or modified cells that recombinantly express CD19) in in vitro culture experiments. A time course of 2, 6, 12, 24, and 48 hours of media and cells were taken from the mixed cell reactions. For example, using Nalm6-CD19WT and Nalm6-CD19KO tumor cell lines, co-cultures supernatants are collected to measure secreted cytokines levels using Enzyme-Linked Immunosorbent assay (ELISA) and flow cytometry-based Biolegend’s Legendplex™ assay. Reprogrammed CAR-T cells secrete cytokines such as IL2, IFNg and TNF only in the supernatants from culture with Nalm6-CD19WT not with Nalm6-CD19KO in a CAR-specific manner. An ELISA confirms increased production of interferon gamma across MOIs when IL-12p70 is co-expressed by the CD19CAR RNV. The greatest differences in interferon gamma production were seen at the lower MOIs.

Flow cytometry of the samples were run to measure T cell proliferation as well as cell killing. There was a dose-dependent observation of increased cell proliferation with increase MOI across all three constructs. In addition, transduced cells when IL-12p70 is co-expressed by the CD19CAR RNV showed an increase in the number of cells division that occurred in the same time frame compared to CD19CAR only expression . Similar effects were observed for cell killing and increased cell killing corresponded to T cell divisions using methodology similar to GFPmiRT.

### Example 13B

In another embodiment, IL12p70 is replaced with IL-2 (construct 14; SEQ ID NO:5) in the CD19CAR expressing RNV. Similar in vitro studies were run as described above with similar results. Western blot and ELISA confirms that IL-2 is expressed along with CD19 CAR in transduced cells. When IL-2 is co-expressed along with CD19 CAR in RNV-transduced T cells, those T cells show increased activity (proliferation), cytokine secretion upon activation by CD19 (interferon gamma), and increased killing of CD19-expressing cells by the transduced T cells in the assays.

### Example 13C

In another embodiment, IL12p70 is replaced with IL-242A (IL-2supmut, construct 13; SEQ ID NO:6) CD19CAR expressing RNV. Similar in vitro studies were run as described above with similar results. Western blot and ELISA confirms that IL-2supmut is expressed along with CD19 CAR in transduced cells. When IL-2supmut is co-expressed along with CD19 CAR in RNV-transduced T cells, those T cells show increased activity (proliferation), cytokine secretion upon activation by CD19 (interferon gamma), and increased killing of CD19-epxressing cells by the transduced T cells. In addition, in vitro studies are done comparing T cell lines 1:1 mixed with Treg cells (primary or MT-2 cell line) transduced with constructs 6, 8, 13 or 14. While cells transduced with constructs 6, 8, and 14 showed activation of Tregs and expression of IL-10, those transduced with IL-2supmut showed levels comparable to baseline confirming that use of IL-2supmut increases activity of CD19CAR+ TH₁ CD4+ and CD8+ T cells while excluding enhanced activity of Treg populations in the same culture.

### Example 13D

In another embodiment, IL12p70 is replaced with IL-7R-CPT (construct 15; SEQ ID NO:4) in the CD19CAR expressing RNV. Interleukin-7 receptor subunit alpha (IL7Ra) is also known as CD antigen CD127, which belongs to the type I cytokine receptor family and type 4 subfamily. IL7Ra /CD127 is expressed on various cell types, including naive and memory T cells and many others. The IL7Ra forms a heterodimer with Iinterleukin-2 receptor subunit gamma (IL2RG) to transmit IL7 signal. However, IL7Ra can acquire cysteine and/or noncysteine mutations to form a homodimer, leading to ligand (IL7) independent signaling events. This ligand independent IL7Ra homodimer (IL7-Ra-CPT) supports continued proliferation and long-term persistence of T cells. Similar in vitro studies were run as described above with similar results. Western blot and ELISA confirm that IL-7Ra-CPT is expressed along with CD19 CAR in transduced cells. When IL-7R-CPT is co-expressed along with CD19 CAR in RNV-transduced T cells, those T cells show increased activity (proliferation), cytokine secretion upon activation by CD19 (interferon gamma), and increased killing of CD19-expressing cells by the transduced T cells similar to the assays run above.

### Example 13E

In another embodiment, IL12p70 is replaced with IL-12 (construct 16) or IL-12 derivatives not related to IL-12p70 in the CD19CAR expressing RNV. Similar in vitro studies were run as described above with similar results. Western blot and ELISA confirms that IL-12 is expressed along with CD19 CAR in transduced cells. When IL-12 is co-expressed along with CD19 CAR in RNV-transduced T cells, those T cells show increased activity (proliferation), cytokine secretion upon activation by CD19 (interferon gamma), and increased killing of CD19-expressing cells by the transduced T cells similar to the assays above.

### Example 13F

In another embodiment, IL12p70 is replaced with cJun (construct 12; SEQ ID NO:7) in the CD19CAR expressing RNV. Expression of c-Jun supports cellular proliferation while overexpression accelerates it. T cells’ dysfunction or exhaustion have been associated with decreased c-Jun levels and overexpression of it restored T effector functions while supporting continued cell cycle progression and long-term persistence. Western blot confirms that cJun is expressed along with CD19 CAR in transduced cells. When cJun is co-expressed along with CD19 CAR in RNV-transduced T cells, those T cells show increased activity (proliferation) and survival, cytokine secretion upon activation by CD19 binding (interferon gamma), and increased killing of CD19-expressing cells by the transduced T cells similar to the assays run above. Increase survival is determined by extending the time course out to 10 days after co-culturing with CD19 positive cells and comparing to results without cJun expression using construct 6.

### Example 13G

In another embodiment, IL12p70 is replaced with IL-15 (construct 9 or 10) in the CD19CAR expressing RNV. IL-15 is a cytokine that stimulates CD8 T cell and natural killer (NK) cell activation, proliferation, and cytolytic activity. Survival signals that maintain memory T cells in the absence of antigen are provided by IL-15 and may be helpful in generating durable CAR T responses. Similar in vitro studies were run as described above with similar results. When IL-15 is co-expressed along with CD19 CAR in RNV-transduced T cells, those T cells show increased activity (proliferation), cytokine secretion upon activation by CD19 (IL2, IFNg and TNF), and increased killing of CD19-expressing cells by the transduced T cells. Additional in vitro studies demonstrate IL-15 expression contribute towards long lasting memory T cell pool in culture.

### Example 13H

### In Vitro Testing of Reverse Orientation Transcribed IL-15 Expressed in a Non-Replicating Retroviral Vector Also Expressing a Chimeric Antigen Receptor Targeted Against CD19

The addition of IL-15 gene engineered to be transcribed in the opposite orientation relative to the CD19 CAR gene in the RNV backbone (construct 9; SEQ ID NO:15) allows for increased expression if IL-15 relative to an IL-15 transgene transcribed in the same orientation as CD19 CAR (construct 10; SEQ ID NO:8). CAR-expressing construct supports sustained and potent T cell activity of the CAR and promotes anti-cancer immune activity of non-CAR transduced immune cells. Primary peripheral T cells or T cell lines (e.g. TALL-104; Jurkat) are transduced with RNV encoding CD19-targeted CAR (pBA9b-CD19CAR) or encoding CD19-targeted CAR that co-expresses IL-15 transgene in either orientation (pBAb-CD19CAR-IL-15, construct 9 or 10) at varying MOIs (e.g., 0.1, 1, and 10). Western blot on samples of the transduced cells are run and confirms a dose-dependent expression of CD19 CAR and for construct 9, increasing IL-15 protein levels corresponding to the increasing MOIs used as well as increased expression when a reverse orientation relative to CD19CAR transgene is used. The RNV-transduced cells are labeled with a proliferation monitoring dye (e.g., cell trace violet). The cells are then mixed with CD19-expressing cells (e.g., NALM-6; primary B cells, or modified cells that recombinantly express CD19) in in vitro culture experiments. A time course of 2, 6, 12, 24, and 48 hours of media and cells were taken from the mixed cell reactions An ELISA confirms increased production of interferon gamma across MOIs when IL-12p70 is co-expressed by the CD19CAR RNV similar to the assays described above.

### Example 13I

In other embodiments, other immune modulators can be engineered to be transcribed in the reverse orientation for increased transgene expression. IL2, IL2supmut, IL12p70 cytokines, IL-7Ra-CPT and cJun as described in Example 13a-f can be used in place of IL-15.

### Example 13J

In other embodiments, pBA-9B RNV backbone can be replaced with a self-inactivating (SIN) LTR (constructs 27; SEQ ID NO:18). With destruction of RNV LTR promoter activity, replacement promoters are used. Intron less EF1a or CMV promoters may be used to express transgenes from the pSIN-RNV. In this example, the human or mouse CD19CAR are expressed from the EF1a promoter encoded within the RNV while still encoding an IRES or 2A sequence to express a kill switch (e.g., yeast cytosine deaminase, thymidine kinase or mutants thereof) (constructs 27). Further, additional transgenes may be expressed from CMV promoter downstream of the kill switch. In constructs 27 and 29; IL-15 cytokine is expressed from a CMV promoter. The CMV promoter and IL-15 gene may be placed in two orientations relative to the transcription direction of CD19CAR (constructs 9 and 10). Similar studies as described above are used to characterize the expression and activity of all transgenes in these constructs.

### Example 13K

In other embodiments; the pBA-9B RNV backbone may be replaced with a self-inactivating (SIN) lentiviral backbone (construct 28; SEQ ID NO:19). All transgene designed including CAR, IRES-kill switch, immune modulators, miRNA target sequences, shRNA, and promoters may be used as described herein. In addition to the RNV sequences (excluding RNV LTR and packaging sequences), woodchuck post-transcriptional regulatory element is used to enhance expression of transgenes (Zufferey et al., “Woodchuck Hepatitis Virus Posttranscriptional Regulatory Element Enhances Expression of Transgenes Delivered by Retroviral Vectors,” Journal of Virology, 73(4):2886-2892, 1999). The same in vitro and in vivo assays described herein to characterize transgene expression and activity is used for this non-replicating SIN lentiviral vector(s).

### Example 13L

### siRNA Constructs

Non-replicating retroviral vectors of the disclosure can be used to modulate the activity of immune cells with expression of engineered siRNA, shRNA or miRNA that switches off or lowers expression of key genes that govern the activity of cells including immune cells. Such targets include genes like PD-1 a central checkpoint in regulating T cell activity, and a key protein in preventing or reducing anti-cancer activity. The in vitro and in vivo assays to measure shRNA-PD-1 expression and transcript knockdown ability is described in U.S. Pats. US20150273029A1. To demonstrate efficient knockdown of PD-1 in primary T cells infected with construct 19 vector at an MOI 0f 10, total RNA is extracted from T cells harvested at d3 post infection. Gene expression of PD-1 is measured by qRT-PCR using RNA polIII promoter transcripts as an internal control for normalization. The relative expression level of PD-1 to naive primary T cells is calculated using ΔΔC(t) method. The data show that at day 3 post-infection, more than 70% of PD-1 is downregulated. The infected cells are cultured in the presence of IL-2 up to 10 days and observe sustained knockdown of PD-1. In parallel, similarly transduced primary T cells are characterized for CD19CAR activity using methods and experiments described above. When PD-1 shRNA is expressed in combination with CD19CAR, 72 hrs-long xCELLigence assay show a doubling of CAR-specific killing and growth inhibition of Nalm6-CD19WT tumor cell line relative to CD19CAR alone.

### Example 14

### In Vivo Testing of IL-12p70 Expressed in a Non-Replicating Retroviral Vector Also Expressing a Chimeric Antigen Receptor Targeted Against CD19

IL-12 is composed of two subunits, IL-12A (p35) and IL-12B (p40), to make a heterodimeric active cytokine IL-12p70. IL-12p70 is naturally produced by antigen presenting cells, including dendritic cells and macrophage, in response to antigenic stimulation. IL-12p70 is a proinflammatory cytokine, enhancing IFNg production and cytotoxic effector function of NK and T cells, promoting Th1 phenotype in CD4 T cells and ADCC activity, and acting as a chemoattractant for dendritic cells and macrophage. These IL-12 activities together can stimulate host anti-tumor activity. The addition of IL-12p70 as a single chain construct (construct 16) to a CAR-expressing construct supports sustained and potent T cell activity of the CAR and promotes anti-cancer immune activity of non-CAR transduced immune cells.

The functional effect of IL-12p70 expressed from the same viral vector expressing a CD19-targeted CAR is tested in vivo by intravenous injection of the recombinant retroviral vectors (pBA9b-CD19CAR, construct 6 or 8, and pBAb-CD19CAR-IL-12p70, construct 16 (human CAR, IL12) [construct 41 is mouse CD19CAR & IL12] in 8-wk-old NSG mice implanted intravenously with luciferized Nalm6 acute lymphoblastic leukemia cells and human PBMC. A dose of 1E6 to 5E8 TU of each vector stock or vehicle control is administered by intravenous injection to each treatment group composed of 10 animals per group for efficacy assessment (tumor burden and survival) and 5 animals per group for immune assessment (flow cytometry). Tumor burden is measured over the duration of the study by periodic imaging of luciferase signal. Survival of control and treatment animals is assessed. The results show treatment with viral vector expressing the CD19CAR (construct 6) reduces tumor burden as measured by luciferase signal and prolongs survival in comparison to control treated animals. CD19CAR-IL-12p70 (construct 16) reduces tumor burden as measured by luciferase signal, prolongs survival in comparison to control and CD19CAR (construct 6) treated animals, and leads to complete absence of detectable tumor in some cases. Flow cytometric analysis is carried out on Nalm6-affected lymph nodes shortly after the initiation of treatment. Results show enhanced T cell activation and degranulation in animals treated with CD19CAR-IL-12p70 (construct 16) compared to CD19CAR (construct 6).

The functional effect of IL-12p70 expressed from the same viral vector expressing a CD19-targeted CAR is tested in vivo by intravenous injection of the recombinant retroviral vectors (pBA9b-mCD19CAR, construct 40 (SEQ ID NO:16) and pBAb-mCD19CAR-IL-12p70, construct 41; SEQ ID NO:13) in immune competent, 8-wk-old Balb/c mice implanted intravenously with luciferized A20 B cell lymphoma cells. A dose of 1E6 to 5E8 TU of each vector stock or vehicle control is administered by intravenous injection to each treatment group composed of 10 animals per group for efficacy assessment (tumor burden and survival) and 5 animals per group for immune assessment (flow cytometry). Tumor burden is measured over the duration of the study by periodic imaging of luciferase signal. Survival of control and treatment animals is assessed. The result show treatment with viral vector expressing the mCD19CAR (construct 40) reduces tumor burden as measured by luciferase signal and prolongs survival in comparison to control treated animals. mCD19CAR-IL-12p70 (construct 41; SEQ ID NO:13) reduces tumor burden as measured by luciferase signal, prolongs survival in comparison to control treated animals, and leads to complete absence of detectable tumor in some cases. Flow cytometric analysis is carried out on A20-affected lymph nodes shortly after the initiation of treatment. Results show enhanced T cell activation and degranulation in animals treated with mCD19CAR-IL-12p70 (construct 41) compared to mCD19CAR (construct 40). In addition, mCD19CAR-IL-12p70-treated animals with no detectable tumor are able to resist rechallenge with A20 cells without any additional treatment. The immune memory associated with resistance to rechallenge is in part associated with the persistence of the mCD19CAR-IL-12p70-transduced T cells, but also with immune learning associated with enhanced inflammation and activation of antigen presentation mediated by IL-12p70 expression. The contribution of immune learning is confirmed by the killing of CAR-expressing cells by activation of the CD kill switch, present in both pBA9b-mCD19CAR and pBAb-CD19CAR-IL-12p70 viral vectors, with flucytosine prior to rechallenge. Following flucytosine IP administration, and confirmation of complete absence of cells expressing CD19CAR-IL-12p70, surviving A20 cured animals were resistant to A20 rechallenge.

### Example 15

### In Vivo Testing of IL-2F42A Expressed in a Non-Replicating Retroviral Vector Also Expressing a Chimeric Antigen Receptor Targeted Against CD19

In another embodiment, IL12p70 is replaced with IL-2 (construct 42; SEQ ID NO:14) in the human and mouse CD19CAR-expressing RNV constructs 14 and 42 respectively). IL-2 is highly inflammatory and acts in the periphery to promote the differentiation of naive T cells into effector and memory T cells. IL-2 is naturally produced by activated CD4+ and CD8+ T cells. IL-2 has been shown to have antitumor activity in several settings, but systemic administration of IL-2 is associated with severe side effects. IL-2 wild-type also acts on Treg cells to suppress immune response. The addition of IL-2F42A, which does not act on Treg cells, to a CAR-expressing construct supports sustained and potent T cell activity of the CAR and promotes anti-cancer immune activity of non-CAR transduced immune cells. In addition, the natural homing capability of CAR expressing T cells to the tumor site of high antigen density for the specific CAR limits IL-2 expression to primarily within the tumor.

Similar in vivo studies were run as described in above with similar results. Results show enhanced T cell activation and degranulation, and increased T cell numbers in Nalm6 tumor-bearing, PBMC-engrafted NSG mice treated with CD19CAR-IL-2F42A(v) (construct 42) compared to CD19CAR. In immune competent animals bearing A20 tumors, results show enhanced T cell activation and degranulation, and increased T cells in mice treated with mCD19CAR-IL-2F42A compared to mCD19CAR. Treatment of A20 tumor-bearing immune competent mice with mCD19CAR-IL-2F42A(v), but not mCD19CAR(v) alone or control treated animals, also leads to complete cures along with immune learning as described herein.

### Example 16

### In Vivo Testing of IL-15-IL-15Ra Expressed in a Non-Replicating Retroviral Vector Also Expressing a Chimeric Antigen Receptor Targeted Against CD19

In another embodiment, IL12p70 is replaced with IL-15-IL-15Ra (constructs 49 & 54 in the human and mouse CD19CAR-expressing RNV. IL-15 is expressed primarily by dendritic cells, monocytes, and macrophage and supports the activation, proliferation, and survival of T and NK cells. IL-15 exists in a membrane bound form complexed with the IL 15Ra receptor. This potent signaling complex can be recapitulated in a soluble form by single chain expression of IL-15 linked to the sushi domain of IL-15Ra (IL-15-IL15Ra). The addition of secreted IL-15-IL-15Ra as a single chain construct to a CAR-expressing construct supports sustained and potent T cell activity of the CAR, differentiation of CAR and non-CAR T cell to long-lived memory T cells, and anti-cancer immune activity of non-CAR transduced immune cells.

Similar in vivo studies were run as described above with similar results. Results show enhanced T cell activation and degranulation in Nalm6 tumor-bearing, PBMC-engrafted NSG mice treated with CD19CAR-IL-15-IL-15Ra (v) (construct 49) compared to CD19CAR. In immune competent animals bearing A20 tumors, results show enhanced T cell activation and degranulation in mice treated with mCD19CAR-IL-15-IL-15Ra (v) (construct 49 compared to mCD19CAR Treatment of A20 tumor-bearing immune competent mice with mCD19CAR-IL-2F42A(v), but not mCD19CAR(v) alone or control treated animals, also leads to complete cures along with immune learning similar to above.

### Example 17

### In Vivo Testing of Interleukin-7 Receptor-a Mutants Expressed in a Non-Replicating Retroviral Vector Also Expressing a Chimeric Antigen Receptor Targeted Against CD19

In another embodiment, IL12p70 is replaced with IL-7Ra-CPT (constructs 15 & 55 – human and mouse CD19CARs) in the CD19CAR-expressing RNV. Interleukin-7 receptor subunit alpha (IL7Ra) is also known as CD antigen CD127, which belongs to the type I cytokine receptor family and type 4 subfamily. IL7Ra/CD127 is expressed on various cell types, including naive and memory T cells and many others. The IL7Ra forms a heterodimer with Iinterleukin-2 receptor subunit gamma (IL2RG) to transmit IL7 signal. However, IL7Ra can acquire cysteine and/or noncysteine mutations to form a homodimer, leading to ligand (IL7) independent signaling events. This ligand independent IL7Ra homodimer supports continued proliferation and long-term persistence of T cells. The addition of mutant IL7Ra construct (construct 15) to a CAR-expressing construct supports sustained proliferation, persistence and potency while promoting anti-cancer immune activity of reprogrammed T cells in vivo.

Similar in vivo studies were run as described above with similar results. Results show enhanced T cell activation and degranulation, and increased T cell numbers in Nalm6 tumor-bearing, PBMC-engrafted NSG mice treated with CD19CAR-IL-7Ra-CPT (construct 15) compared to CD19CAR. In immune competent animals bearing A20 tumors, results show enhanced T cell activation and degranulation, and increased T cells in mice treated with mCD19CAR-IL-7Ra-CPT (construct 55 compared to mCD19CAR Treatment of A20 tumor-bearing immune competent mice with mCD19CAR-IL-7Ra-CPT(v), but not mCD19CAR(v) alone or control treated animals, also leads to complete cures along with immune learning similar to above.

### Example 18

### In Vivo Testing of c-Jun Expressed in a Non-Replicating Retroviral Vector Also Expressing a Chimeric Antigen Receptor Targeted Against CD19

In another embodiment, IL12p70 is replaced with c-Jun (Construct 12; SEQ ID NO:7) in the human and mouse CD19CAR-expressing RNV. c-Jun is a protein encoded by the JUN gene. c-Jun, in combination with c-Fos, forms the AP-1 early response transcription factor. c-jun transcription is autoregulated by its own product thus it prolongs its signals from extracellular stimuli. Expression of c-Jun supports cellular proliferation while overexpression accelerates it. T cells’ dysfunction or exhaustion have been associated with decreased c-Jun levels and overexpression of it restored T effector functions while supporting continued cell cycle progression and long-term persistence. The addition of c-Jun construct (construct xx) to a CAR-expressing construct supports effector functions, sustained proliferation, persistence and potency while promoting anti-cancer immune activity of reprogrammed T cells in vivo sustained proliferation, persistence and potency while promoting anti-cancer immune activity of reprogrammed T cells in vivo.

Similar in vivo studies were run as described above with similar results. Results show enhanced T cell activation and degranulation, and increased T cell numbers in Nalm6 tumor-bearing, PBMC-engrafted NSG mice treated with CD19CAR-c-Jun compared to CD19CAR. In immune competent animals bearing A20 tumors, results show enhanced T cell activation and degranulation, and increased T cells in mice treated with mCD19CAR-c-Jun compared to mCD19CAR. Treatment of A20 tumor-bearing immune competent mice with mCD19CAR-c-Jun(v), but not mCD19CAR(v) alone or control treated animals, also leads to complete cures along with immune learning similar to above.

### Example 19

### In Vivo Testing of a Short Hairpin RNA Against Checkpoint Inhibitor PD-1 (shPD-1) Expressed in a Non-Replicating Retroviral Vector Also Expressing a Chimeric Antigen Receptor Targeted Against CD19

In another embodiment, IL12p70 is replaced with a short hairpin RNA against checkpoint inhibitor PD-1 (construct 19; SEQ ID NO:9) in the CD19CAR-expressing RNV. PD-1 is expressed by T cells after activation and is an indicator of T cell exhaustion. PD-1 suppresses T cell inflammatory activity and promotes T cell apoptosis. T cells’ dysfunction or exhaustion can be reversed by blocking PD-1 activity. PD-1 checkpoint blockade with therapeutic antibodies has been shown clinically efficacy in certain cancer indications by supporting T cell killing of cancer cells. The addition of shPD-1 (construct 19) to a CAR-expressing construct supports sustained activity of the CAR-expressing T cell, including survival, proliferation, and cancer cell killing.

Similar in vivo studies were run as described above with similar results. Results show enhanced T cell activation and degranulation, and increased T cell numbers in Nalm6 tumor-bearing, PBMC-engrafted NSG mice treated with CD19CAR-shPD-1 (construct 19) compared to CD19CAR. In immune competent animals bearing A20 tumors, results show enhanced T cell activation and degranulation, and increased T cells in mice treated with mCD19CAR-shPD-1 (construct m-shPD-1) compared to mCD19CAR (construct 40; SEQ ID NO:16). Treatment of A20 tumor-bearing immune competent mice with mCD19CAR-shPD-1(v), but not mCD19CAR(v) alone or control treated animals, also leads to complete cures along with immune learning similar to above.

### Example 20

BCMA is a mature plasma cell marker that is expressed minimally elsewhere, and has been used successfully as a CAR recognized target in the treatment of multiple myeloma (Kochenderfer JN. et al. N Engl J Med., 380(18):1726-1732, May 2, 2019). Anti-BCMA CARS are used to show the efficacy of the in vivo CAR Delivery system for antigens other than CD19.

### Construction and in Vitro Testing of a-BCMA CAR Vectors with a Suicide Gene and De-Targeting miRNA Targets for B Cells and Monocytes

The structure of the vector is as for anti-CD19 CARs with the substitution of known anti-BCMA CAR sequences such as the humanized Vh only FHVH33-CD8BBZ (Lam et al., Nature Communic., 2020) to make pBA-9b-BCMACAR1.FHVH33-CD8BBZ.miRT663A-223.

A corresponding codon optimized DNA nucleic acid sequence is:

ATGGCCCTGCCCGTGACCGCCCTGCTGCTGCCCCTGGCCCTGCTGCTGCA
CGCCGCCAGGCCCGAGGTGCAGCTGCTGGAGAGCGGCGGCGGCCTGGTGC
AGCCCGGCGGCAGCCTGAGGCTGAGCTGCGCCGCCAGCGGCTTCACCTTC
AGCAGCTACGCCATGAGCTGGGTGAGGCAGGCCCCCGGCAAGGGCCTGGA
GTGGGTGAGCAGCATCAGCGGCAGCGGCGACTACATCTACTACGCCGACA
GCGTGAAGGGCAGGTTCACCATCAGCAGGGACATCAGCAAGAACACCCTG
TACCTGCAGATGAACAGCCTGAGGGCCGAGGACACCGCCGTGTACTACTG
CGCCAAGGAGGGCACCGGCGCCAACAGCAGCCTGGCCGACTACAGGGGCC
AGGGCACCCTGGTGACCGTGAGCAGCTTCGTGCCCGTGTTCCTGCCCGCC
AAGCCCACCACCACCCCCGCCCCCAGGCCCCCCACCCCCGCCCCCACCAT
CGCCAGCCAGCCCCTGAGCCTGAGGCCCGAGGCCTGCAGGCCCGCCGCCG
GCGGCGCCGTGCACACCAGGGGCCTGGACTTCGCCTGCGACATCTACATC
TGGGCCCCCCTGGCCGGCACCTGCGGCGTGCTGCTGCTGAGCCTGGTGAT
CACCCTGTACTGCAACCACAGGAACAAGAGGGGCAGGAAGAAGCTGCTGT
ACATCTTCAAGCAGCCCTTCATGAGGCCCGTGCAGACCACCCAGGAGGAG
GACGGCTGCAGCTGCAGGTTCCCCGAGGAGGAGGAGGGCGGCTGCGAGCT
GAGGGTGAAGTTCAGCAGGAGCGCCGACGCCCCCGCCTACCAGCAGGGCC
AGAACCAGCTGTACAACGAGCTGAACCTGGGCAGGAGGGAGGAGTACGAC
GTGCTGGACAAGAGGAGGGGCAGGGACCCCGAGATGGGCGGCAAGCCCAG
GAGGAAGAACCCCCAGGAGGGCCTGTACAACGAGCTGCAGAAGGACAAGA
TGGCCGAGGCCTACAGCGAGATCGGCATGAAGGGCGAGAGGAGGAGGGGC
AAGGGCCACGACGGCCTGTACCAGGGCCTGAGCACCGCCACCAAGGACAC
CTACGACGCCCTGCACATGCAGGCCCTGCCCCCCAGG; or

(SID50) a codon optimized DNA nucleic acid sequence corresponding to a CAR peptide in WO 2015/158671 to make pBA-9b-BCMACAR2.SID50.miRT663A-223

ATGGCCCTGCCCGTGACCGCCCTGCTGCTGCCCCTGGCCCTGCTGCTGCA
CGCCGCCAGACCCCAGGTGCAGCTGGTGCAGAGCGGCGCCGAGGTGAAGA
AGCCCGGCGCCAGCGTGAAGGTGAGCTGCAAGGCCAGCGGCTACAGCTTC
CCCGACTACTACATCAACTGGGTGAGACAGGCCCCCGGCCAGGGCCTGGA
GTGGATGGGCTGGATCTACTTCGCCAGCGGCAACAGCGAGTACAACCAGA
AGTTCACCGGCAGAGTGACCATGACCAGAGACACCAGCATCAACACCGCC
TACATGGAGCTGAGCAGCCTGACCAGCGAGGACACCGCCGTGTACTTCTG
CGCCAGCCTGTACGACTACGACTGGTACTTCGACGTGTGGGGCCAGGGCA
CCATGGTGACCGTGAGCAGCGGCGGCGGCGGCAGCGGCGGCGGCGGCAGC
GGCGGCGGCGGCAGCGACATCGTGATGACCCAGACCCCCCTGAGCCTGAG
CGTGACCCCCGGCCAGCCCGCCAGCATCAGCTGCTGGAGCAGCCAGAGCC
TGGTGCACAGCAACGGCAACACCTACCTGCACTGGTACCTGCAGAAGCCC
GGCCAGAGCCCCCAGCTGCTGATCTACAAGGTGAGCAACAGATTCAGCGG
CGTGCCCGACAGATTCAGCGGCAGCGGCAGCGGCACCGACTTCACCCTGA
AGATCAGCAGAGTGGAGGCCGAGGACGTGGGCATCTACTACTGCAGCCAG
AGCAGCATCTACCCCTGGACCTTCGGCCAGGGCACCAAGCTGGAGATCAA
GACCACCACCCCCGCCCCCAGACCCCCCACCCCCGCCCCCACCATCGCCA
GCCAGCCCCTGAGCCTGAGACCCGAGGCCTGCAGACCCGCCGCCGGCGGC
GCCGTGCACACCAGAGGCCTGGACTTCGCCTGCGACATCTACATCTGGGC
CCCCCTGGCCGGCACCTGCGGCGTGCTGCTGCTGAGCCTGGTGATCACCC
TGTACTGCAAGAGAGGCAGAAAGAAGCTGCTGTACATCTTCAAGCAGCCC
TTCATGAGACCCGTGCAGACCACCCAGGAGGAGGACGGCTGCAGCTGCAG
ATTCCCCGAGGAGGAGGAGGGCGGCTGCGAGCTGAGAGTGAAGTTCAGCA
GAAGCGCCGACGCCCCCGCCTACCAGCAGGGCCAGAACCAGCTGTACAAC
GAGCTGAACCTGGGCAGAAGAGAGGAGTACGACGTGCTGGACAAGAGAAG
AGGCAGAGACCCCGAGATGGGCGGCAAGCCCAGAAGAAAGAACCCCCAGG
AGGGCCTGTACAACGAGCTGCAGAAGGACAAGATGGCCGAGGCCTACAGC
GAGATCGGCATGAAGGGCGAGAGAAGAAGAGGCAAGGGCCACGACGGCCT
GTACCAGGGCCTGAGCACCGCCACCAAGGACACCTACGACGCCCTGCACA
TGCAGGCCCTGCCCCCCAGA; or

(SID56) a codon optimized DNA nucleic acid sequence corresponding to a CAR peptide in WO 2015/158671, to make pBA-9b-BCMACAR3.SID 56.miRT663A-223

ATGGCCCTGCCCGTGACCGCCCTGCTGCTGCCCCTGGCCCTGCTGCTGCA
CGCCGCCAGACCCCAGGTGCAGCTGGTGCAGAGCGGCGCCGAGGTGAAGA
AGCCCGGCGCCAGCGTGAAGGTGAGCTGCAAGGCCAGCGGCTACAGCTTC
CCCGACTACTACATCAACTGGGTGAGACAGGCCCCCGGCCAGGGCCTGGA
GTGGATGGGCTGGATCTACTTCGCCAGCGGCAACAGCGAGTACAACCAGA
AGTTCACCGGCAGAGTGACCATGACCAGAGACACCAGCATCAACACCGCC
TACATGGAGCTGAGCAGCCTGACCAGCGAGGACACCGCCGTGTACTTCTG
CGCCAGCCTGTACGACTACGACTGGTACTTCGACGTGTGGGGCCAGGGCA
CCATGGTGACCGTGAGCAGCGGCGGCGGCGGCAGCGGCGGCGGCGGCAGC
GGCGGCGGCGGCAGCGACATCGTGATGACCCAGACCCCCCTGAGCCTGAG
CGTGACCCCCGGCCAGCCCGCCAGCATCAGCTGCTGGAGCAGCCAGAGCC
TGGTGCACAGCAACGGCAACACCTACCTGCACTGGTACCTGCAGAAGCCC
GGCCAGAGCCCCCAGCTGCTGATCTACAAGGTGAGCAACAGATTCAGCGG
CGTGCCCGACAGATTCAGCGGCAGCGGCAGCGGCACCGACTTCACCCTGA
AGATCAGCAGAGTGGAGGCCGAGGACGTGGGCATCTACTACTGCAGCCAG
AGCAGCATCTACCCCTGGACCTTCGGCCAGGGCACCAAGCTGGAGATCAA
GACCACCACCCCCGCCCCCAGACCCCCCACCCCCGCCCCCACCATCGCCA
GCCAGCCCCTGAGCCTGAGACCCGAGGCCTGCAGACCCGCCGCCGGCGGC
GCCGTGCACACCAGAGGCCTGGACTTCGCCTGCGACATCTACATCTGGGC
CCCCCTGGCCGGCACCTGCGGCGTGCTGCTGCTGAGCCTGGTGATCACCC
TGTACTGCAAGAGAGGCAGAAAGAAGCTGCTGTACATCTTCAAGCAGCCC
TTCATGAGACCCGTGCAGACCACCCAGGAGGAGGACGGCTGCAGCTGCAG
ATTCCCCGAGGAGGAGGAGGGCGGCTGCGAGCTGAGAGTGAAGTTCAGCA
GAAGCGCCGACGCCCCCGCCTACCAGCAGGGCCAGAACCAGCTGTACAAC
GAGCTGAACCTGGGCAGAAGAGAGGAGTACGACGTGCTGGACAAGAGAAG
AGGCAGAGACCCCGAGATGGGCGGCAAGCCCAGAAGAAAGAACCCCCAGG
AGGGCCTGTACAACGAGCTGCAGAAGGACAAGATGGCCGAGGCCTACAGC
GAGATCGGCATGAAGGGCGAGAGAAGAAGAGGCAAGGGCCACGACGGCCT
GTACCAGGGCCTGAGCACCGCCACCAAGGACACCTACGACGCCCTGCACA
TGCAGGCCCTGCCCCCCAGA.

The resulting plasmids are used to generate the corresponding infectious RNV preparations. Infectious vector is used to test activity of CARs in vitro and in vivo as described below.

### In Vitro Testing

First CAR T cells are created in vitro by transduction of PBMC with appropriate T cell stimulation as described herein. The transduced cells are characterized and result in 20-80% T cell transduction. These cells are then used for in vitro testing

### Degranulation Assay (CD107a Mobilization)

T-cells are incubated in 96-well plates (40,000 transduced cells/well), together with an equal amount of cells expressing or not expressing the BCMA protein. Co-cultures are maintained in a final volume of 100 µl of X-Vivo™-15 medium (Lonza) for 6 hours at 37° C. with 5% CO₂. CD107a staining is performed during cell stimulation, by the addition of a fluorescent anti-CD107a antibody (APC conjugated, from Miltenyi Biotec) at the beginning of the co-culture, together with 1 µg/ml of anti-CD49d (BD Pharmingen), 1 ug/ml of anti-CD28 (Miltenyi Biotec), and 1x Monensin solution (eBioscience). After the 6 h incubation period, cells are stained with a fixable viability dye (eFluor 780, from eBioscience) and fluorochrome-conjugated anti-CD8 (PE conjugated Miltenyi Biotec) and analyzed by flow cytometry. The degranulation activity is determined as the % of CD8+/CD107a+ cells, and by determining the mean fluorescence intensity signal (MFI) for CD107a staining among CD8+ cells. Degranulation assays are carried out at east 24 h after PBMC/T cell transduction.

Anti-BCMA CARs T cells are active against BCMA expressing cancer cells expressing BCMA (RPMI8226 and NCI-H929), while no activity is detected in CARs T cells wherein said CAR is against an irrelevant target (e.g., mouse CD19) or in T cells transduced with GFP. No/background activity is seen with either type of transduced T cells when the target is BCMA negative (K562 cells).

### Example 21

### In Vivo Testing of BCMA CAR

Female NOD-Cg-Prkdcscid IL2rgtm1Wjl/SzJ (NSG; Jackson Laboratories) mice receive subcutaneous (s.c.) injections of 0.2 mL of a 5E7 cells/mL suspension containing E7 BCMA+ RPMI-8226 MM tumor cells to establish s.c. xenografts. At approximately 10-15 days post tumor implantation, mice with xenografts receive a single intravenous (i.v.) injection of 0.2 mL of a cell suspension containing E5 to E8 of human T cells. At day 18 approximately, mice are randomized to groups of 10 mice, when mean tumor volumes of 96 +/-16 mm³ and after confirming engraftment of human T cells. Groups receive iv administration of CAR encoding vectors or control vectors at doses of E3, E4, E5, E6, E7, E8 or E9 TU’s of vector. An optional additional positive control group receives 1 mg/kg of bortezomib (Velcade) i.v. twice weekly, for 4 weeks. Mice are monitored until approximately day 100 for tumor growth. Tumor growth inhibition, compared to controls, is observed in some groups of mice and not in others depending on the dose and vector preparation used, and the most effective dose and vector are identified.

### Example 22

### In Vivo Testing of CD8-Targeted/Pseudotyped Particles Containing Non-Replicating Retroviral Vector Expressing a Chimeric Antigen Receptor Targeted Against BCMA and Expressing IL-12p70

Non-replicating retroviral particles encoding for aBCMA-CAR and single chain IL12p70 are pseudotyped for in vivo transduction of CD8+ T cells through incorporation of an anti-CD8a scFv into the measles envelop protein (construct 52; SEQ ID NO:20) and the Truncated measles F protein that causes fusion once the virus has “docked” via the “H” protein hybrid. This allows for exquisite transduction of mainly CD8+ T cells. This avoids potential transduction of other proliferating cell types, including tumor cells, non-CD8+ immune cells, and proliferating liver and endothelial cells. Efficient targeting of CD8+ cells also increases the relative number of transduced CD8+ cells since untargeted cells no longer act a sink for vector deposition.

The functional effect of CD8-targeted/pseudotyped particles containing non-replicating retroviral vector expressing a chimeric antigen receptor targeted against BCMA and expressing IL-12p70 is tested in vivo by intravenous injection of the recombinant retroviral vectors BCMA-CAR-IL-12p70, made from the amphotropic producer line and the producer line with the hybrid envelopes, in 8-wk-old NSG mice implanted subcutaneous with a BCMA positive tumor cell line and intravenously with human PBMC. A dose of 1E6 to 5E8 TU of each vector stock or vehicle control is administered by intravenous injection to each treatment group composed of 10 animals per group for efficacy assessment (tumor volume). Tumor volume is measured over the duration of the study. The results show treatment with viral vector expressing the BCMA-CAR-IL-12p70 reduces tumor volume in comparison to control treated animals. CD8-PSMA-CAR-IL-12p70 reduces tumor volume in comparison to control and non-targeted PSMA-CAR-IL-12p70 treated animals, and leads to complete absence of measurable tumor in some cases. Flow cytometric analysis of peripheral blood 2 days after intravenous injection of the recombinant retroviral vectors confirms that -CAR-IL-12p70 transduction is limited to CD8+ cells while non-targeted BCMA-CAR-IL-12p70 transduction is detectible across a broad range of immune cell populations, including CD8+, CD4+, B cells and monocytes. In addition, the percent of CD8+ cells transduced is higher with targeted vector- compared to non-targeted vector.

### Example 23

miRNA expression levels were quantified from primary biopsies from non-Hodgkin’s lymphoma (represented by DLBCL) and multiple myeloma patients in addition to CD4+ and CD8+ peripheral blood mononuclear cells from healthy donors. Samples were extracted for total RNA and then processed for miRNA isolation using Illumina Small RNA-Seq library construction. Libraries were sequenced via Illumina NextSeq sequencing at approximately 10,000,000 reads per sample, using single end reads, minimum read length 1×75 bp. Sequencing results were processed and analyzed in R to determine miRNA expression across samples. Target miRNAs and their corresponding Target Sequence were identified by two methods differential and ranked analysis of the resulting miRNA expression profiles from sequencing.

### Non-Hodgkin’s Lymphoma (NHL) miRNA Candidate Analysis

Tables 2 and 3 show the results of two different methods (differential or ranked) to calculate and identify the miRNA expressed in DLBCL that are not expressed well in T cells. Top candidates are highlighted in both tables. Table 5 shows common targets identified by both methods.

TABLE 3

Differential Expression Method (The R isoMirs package was used to perform differential expression analysis on count data)
Differential Expression- DLBCL versus T Cells

| gene | baseMean | log2FoldChange | stat | pvalue |
|---|---|---|---|---|
| hsa-miR-223-3p | 17208.1 | -5.18 | -7.01 | 1.16615E-12 |
| hsa-miR-143-3p | 13613.4 | -8.50 | -11.39 | 2.2634E-30 |
| hsa-miR-200c-3p | 4081.4 | -4.43 | -6.60 | 2.0456E-11 |
| hsa-miR-182-5p | 2815.2 | -6.92 | -7.91 | 1.33546E-15 |
| hsa-miR-125b-5p | 1520.4 | -4.76 | -6.41 | 7.34098E-11 |
| hsa-miR-100-5p | 1454.3 | -5.34 | -5.07 | 1.98865E-07 |
| hsa-miR-183-5p | 1096.4 | -7.11 | -7.00 | 1.2708E-12 |
| hsa-miR-10b-5p | 810.3 | -7.35 | -7.58 | 1.76381E-14 |
| hsa-miR-141-3p | 680.4 | -4.74 | -5.13 | 1.4357E-07 |
| hsa-miR-30a-5p | 358.3 | -5.87 | -10.13 | 1.96896E-24 |
| hsa-miR-144-5p | 321.5 | -6.41 | -4.23 | 1.15698E-05 |

TABLE 4

: Rank Percentile Comparison Method (Based on the RPKM matrix tables, all miRNA expression grouped by cell types was ranked ordered); Median RPKM expression values for each miRNA are shown in the table below for the two groups together with the log2FC of these values
Top 90% DLBCL Expressors vs lowest percentage of T-Cells

| gene | DLBCL | T Cells | Log2FoldChange |
|---|---|---|---|
| hsa-miR-143-3p | 5037.3 | 5.5 | 9.8 |
| hsa-miR-223-3p | 3659.4 | 169.8 | 4.4 |
| hsa-miR-126-3p | 836.6 | 40.6 | 4.4 |
| hsa-mir-21 | 616.7 | 108.6 | 2.5 |
| hsa-miR-486-5p | 533.6 | 119.4 | 2.2 |
| hsa-miR-182-5p | 418.6 | 10.9 | 5.3 |
| hsa-mir-223 | 315.6 | 7.1 | 5.5 |

TABLE 5

Common miRNA targets from differential expression and Ranked comparison methods

| Differential Expression- DLBCL versus T Cells | | | | | Median RPKM expression values | |
|---|---|---|---|---|---|---|
| gene | baseMean | log2FoldChange | stat | pvalue | DLBCL | T Cells |
| hsa-miR-223-3p | 17208.1 | -5.18 | -7.01 | 1.16615E-12 | 3659.397 | 169.820 |
| hsa-miR-143-3p | 13613.4 | -8.50 | -11.39 | 2.2634E-30 | 5037.3335 | 5.498 |
| hsa-miR-182-5p | 2815.2 | -6.92 | -7.91 | 1.33546E-15 | 418.592 | 10.911 |
| hsa-miR-10b-5p | 810.3 | -7.35 | -7.58 | 1.76381E-14 | 20.803 | 2.121 |
| hsa-miR-141-3p | 680.4 | -4.74 | -5.13 | 1.4357E-07 | 145.7505 | 2.419 |

### Multiple Myeloma (MM) miRNA Candidate Analysis

Tables 6 and 7 show the results of two different methods (differential or ranked) to calculate and identify the top miRNA expressed in MM that are not expressed well in T cells. Top candidates are highlighted in both tables. Table 6 shows common targets identified by both methods.

TABLE 6

Differential Expression Method (The R isoMirs package was used to perform differential expression analysis on count data)
Differential Expression- MM versus T Cells

| gene | baseMean | log2FoldChange | stat | pvalue |
|---|---|---|---|---|
| hsa-miR-223-3p | 20333.5 | -5.2 | -11.2 | 3.4E-29 |
| hsa-miR-486-5p | 16879.1 | -5.1 | -10.6 | 1.7E-26 |
| hsa-miR-143-3p | 11126.4 | -8.0 | -14.6 | 9.1E-49 |
| hsa-miR-144-5p | 1401.9 | -8.4 | -7.5 | 2.4E-14 |
| hsa-miR-182-5p | 1022.1 | -5.2 | -7.3 | 1E-13 |
| hsa-miR-183-5p | 664.8 | -6.2 | -7.2 | 2.4E-13 |
| hsa-miR-30a-5p | 333.6 | -5.5 | -9.6 | 3.6E-22 |

TABLE 7

Rank Percentile Comparison Method (Based on the RPKM matrix tables, all miRNA expression grouped by cell types were rank ordered); Median RPKM expression values for each miRNA are shown in the table below for the two groups together with the log2FC of these values
Top 90% MM Expressors vs lowest percentage of T-Cells

| gene | MM | T Cells | Log2FoldChange |
|---|---|---|---|
| hsa-miR-223-3p | 6569.8 | 169.8 | 5.3 |
| hsa-miR-148a-3p | 3779.2 | 539.3 | 2.8 |
| hsa-miR-143-3p | 2917.7 | 5.5 | 9.1 |
| hsa-miR-486-5p | 2022.7 | 119.4 | 4.1 |
| hsa-mir-223 | 590.6 | 7.1 | 6.4 |
| hsa-miR-451a | 564.7 | 11.3 | 5.6 |
| hsa-mir-21 | 507.5 | 108.6 | 2.2 |
| hsa-miR-126-3p | 481.2 | 40.6 | 3.6 |

TABLE 8

Common miRNA targets from differential expression and Ranked comparison methods

| Differential Expression- MM versus T Cells | | | | | Median RPKM expression values | |
|---|---|---|---|---|---|---|
| gene | baseMean | log2FoldChange | stat | pvalue | MM | T Cells |
| hsa-miR-223-3p | 20333.5 | -5.2 | -11.2 | 3.4E-29 | 6569.8 | 169.8 |
| hsa-miR-486-5p | 16879.1 | -5.1 | -10.6 | 1.7E-26 | 2022.7 | 119.4 |
| hsa-miR-143-3p | 11126.4 | -8.0 | -14.6 | 9.1E-49 | 2917.7 | 5.5 |

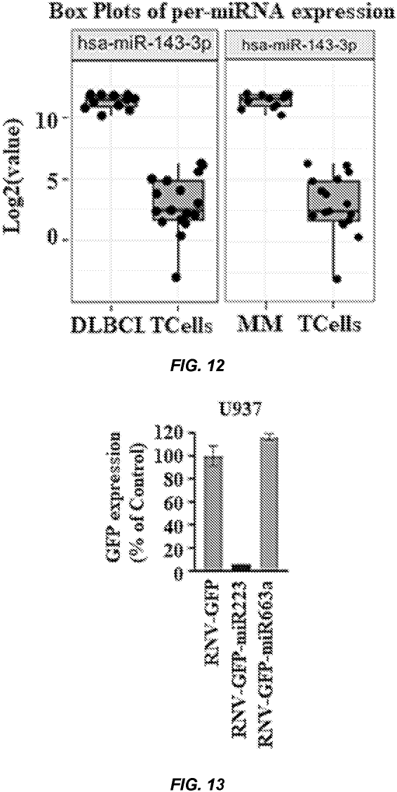

FIG. 12 shows an example box plot of ideal miRNA characteristics whose corresponding target sequences can be used in gene therapy constructs to reduce off target expression of transgenes. There is overlap between strong miRNA candidate in NHL and MM with miRNAs hsa-miR-223-3p and hsa-miR-143-3p identified to reduce off target expression in those cancers. Candidate miRNAs and miRNA target sequences encoded in RNVs were confirmed to have biological activity in vitro. For example, FIG. 13 shows the effect of including a miRNA target sequence in an RNV vector. The target sequence for the miR223-3p was inserted into a GFP vector to give the sequence pBA-9B-GFPmiR223-3pB- 4TX (construct 7; SEQ ID NO:2) and used to make infectious vector. miR223-3p is a microRNAs which is produced at significant concentrations only in monocytic or myeloid cells. FIG. 13 shows that in the U937 monocytic cell line, a 100 fold reduction in GFP expression in the GFPmiR223 infected cell line, compared to the two other vectors. All three vectors produced equivalent amounts of GFP in HT1080 fibrosarcoma cells or other non-monocytic cells.

### Example 24

### Creation of the Amphotropic Packaging and Producer Cell Lines for Large-Scale Vector Production of Retroviral Vectors Encoding Human Adenosine Deaminase (ADA) or Vectors and Clinical Application with Consideration to Improved Safety and High Titer

### Plasmid Constructions

### Retroviral Vector Constructs

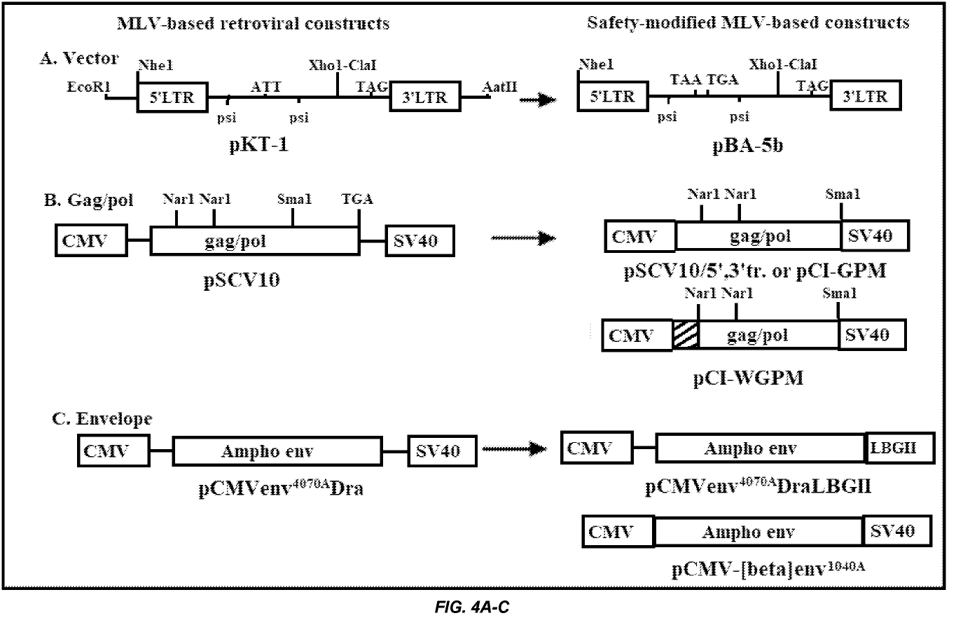

The original N2-derived retroviral vector pKT-1 (Patent Applications WO 91/06852 and WO 92/05266), and all its safety modifications, are summarized in FIGS. 4A-C. Retroviral vector pCBβ-gal is derived from pKT-1 and codes for the β galactosidase and neo^{r} genes. The reduced homology vector, pBA-5b, is a result of several safety modifications incorporated into pKT-1. Vector pKT-1, which already contained the modification ATT in place of the normal ATG start site of gag, is modified to contain two stop codons in the extended packaging signal (Ψ+); the ATT modified start site was changed to the stop codon TAA, and an additional TGA stop codon was inserted 21 nt downstream. All extraneous MLV-derived retroviral sequences upstream of the 5′ LTR, downstream of the 3′ LTR, and between the polypurine tract and the stop codon of env, are eliminated, creating the vector pBA-9b.

### MoMLV-Derived Gag/Pol Constructs

Safety-modifications on the original MoMLV-derived gag/pol plasmid pSCV10 (patent applications WO 91/06852, WO 92/05266) were carried out to reduce sequence homology to the retroviral vector and env expression constructs (FIG. 4B). The expression cassette pCI-WGPM contains degenerate code in approximately the first 400 nt of the coding region for gag, as well as deletions of all 5′ and 3′ untranslated sequences. In addition, the sequence coding for the last 28 amino acids of the pol gene is deleted, resulting in a truncated integrase gene. Plasmids pCI-GPM and pSCV10/5′,3′tr. contain the same gag/pol cDNA as pCI-WGPM except that the 5′ area of gag contains the native sequence.

### Envelope Constructs

To reduce sequence overlap in the gag/pol and retroviral vector plasmids, the original 4070A-derived amphotropic expression plasmid pCMVenv^{am}Dra (Patent Application WO 91/06852) was used to generate two plasmids (FIG. 4C) with either all 3′ untranslated sequences deleted after the env stop codon (pCMVenv^{am}DraLBGH), or all 3′ and 5′ untranslated sequences deleted (pCMV-β/env^{am}) . The xenotropic retroviral envelope expression cassette pCMV^{xeno} was derived from NZB9-1 and the amphotropic envelope expression cassette pMLPenv^{am} was derived from 4070A.

### Parental Cells

Human kidney 293 cells (ATCC CRL 1573), human fibrosarcoma HT-1080 cells (ATCC CCL 121), canine sarcoma D-17 cells (ATCC CRL 8468) and retroviral packaging and producer cell lines derived from these parent cells are maintained in DMEM (Irvine Scientific, CA) supplemented with 10% γ-irradiated defined fetal bovine serum (FBS, Hyclone Laboratories Inc., UT), 20 mM Hepes (Irvine Scientific, CA), 1X non-essential amino acids and 1 mM sodium pyruvate. Parent cell lines used to generate clinical vector producing cell lines are banked and tested in accordance with FDA guidelines for origin (i.e., isoenzyme analysis and karyotyping), absence of expressed retroviral sequences and adventitious agents including mycoplasma, bacteria, fungus and viruses.

### Production of VSV-G Pseudotyped Supernatant

Large-scale production of concentrated VSV-G (vesicular stomatitis virus glycoprotein) pseudotyped vector supernatant (G-supernatant) is performed as outlined by Yee et al. with some minor modifications. Briefly, HA-LB packaging cells (Table 9) are plated into T225 flasks at 1 × 10⁷ cells/flask. After 12 to 20 hours the cells are CaPO₄-transfected with the VSV-G coding plasmid pMLP-G and the respective retroviral vector using the ProFection kit (Promega Corp., WI). Following incubation with the DNA precipitate for 6-8 h, the DNA suspension is removed and fresh media added. After 12 to 20 hours, the supernatant are collected and fresh media applied. Four to five repeat collections are made and the G-supernatant pooled, filtered (0.45 pm) and concentrated by centrifugation at 9000 g and 8° C. for 8-18 hours. Pellets are resuspended in a small volume of fresh media, aliquoted, frozen under liquid nitrogen, and stored at -70° C. This concentrated viral supernatant is then evaluated for titer by transfer of expression (TOE, see below) and PCR titer analysis before carrying out high m.o.t. generation of producer pools and clones.

### Example 25

### Generation and Analysis of MLV-Based Packaging and Producer Cell Lines

### Packaging Cell Lines

Generation of the PCLs DA, 2A, HX, and 2X is described in detail (Pat. Nos. WO 91/06852 and WO92/05266) and the procedure further refined for the generation of the PCLs 2A-LB, HA-LB, HAII, DAII, and DAwob. In general, retroviral gag/pol and env expression plasmids are sequentially introduced into cells by CaPO₄-mediated co-transfection with a phleomycin or methotrexate marker plasmid followed by the appropriate selection for 2 weeks. Selected gag/pol intermediate pools are analyzed for p30 expression and subsequently dilution cloned into 96-well plates according to standard protocols. Gag/pol intermediate clones are analyzed for p30 expression in a Western blot (polyclonal goat anti-p30 antibodies, kindly provided by J. Elder) as well as for titer potential by transduction with a retroviral vector encoding amphotropic env plus a selectable marker and titering the vector produced. Clones with the highest titer potential are co-transfected with a retroviral env expression plasmid and a marker, transfected cells selected, dilution cloned and PCL clones are analyzed for gp70 expression in a Western blot (polyclonal goat anti-gp70 antibodies; Quality Biotech, MD) and for titer potential. The titer potential is tested by several rounds of transduction using several retroviral vector constructs into PCLs at a high ratio of vector to PCL in order to test the limits of the packaging capacity.

TABLE 9

Summary of Characteristic Features of MLV-Based Packaging Cell Lines

| | | | | | | | |
|---|---|---|---|---|---|---|---|
| PCL clone | Gag/pol construct | Envelope construct | Parent Line | Configuration (see FIG. 2 ) | Maximum pool titer (CFU/ml) | Maximum clone titer (CFU/ml) | Research or clinical trials |
| DA^{b} | pSCV10 | pCMVerv DRe | 0–17 | A | 5 × 10⁶ | 2 × 10⁷ | Clinical^{c} |
| 2A^{b} | pSCV10 | pMLPenv | 298 | A | 8 × 10⁶ | 1 × 10⁷ | Res |
| HX^{b} | pSCV10 | pCMVxeno | HT-1080 | A | 4 × 10⁶ | 4 × 10⁸ | Res |
| 2X^{b} | pSCV10 | pCMVxeno | 293 | A | 7 × 10⁶ | 2 × 10⁸ | Res |
| 2A-LB | pSCV10 | pCMVenv DraLBGH | 293 | B | 8 × 10⁶ | 5 × 10⁷ | Res |
| HA-LB | pSCV10 | pCMVenv DraLBGH | HT-1080 | B | 2 × 10⁷ | 2 × 10⁸ | Res |
| HAII | pSCV10/6′.3′tr. | pCMV.penv | HT-1080 | C | 8 × 10⁶ | 1 × 10⁷ | Clinical^{d} |
| DAII | pCl-GPM | pCMV.penv | D-17 | C | 8 × 10⁶ | n.d. | Res |
| DAwob | pCl-WGPM | pCMV.penv | D-17 | D | 5 × 10⁸ | n.d. | Res |

### Example 26

### Engineered Amphotropic Env for Cell-Specific Targeting

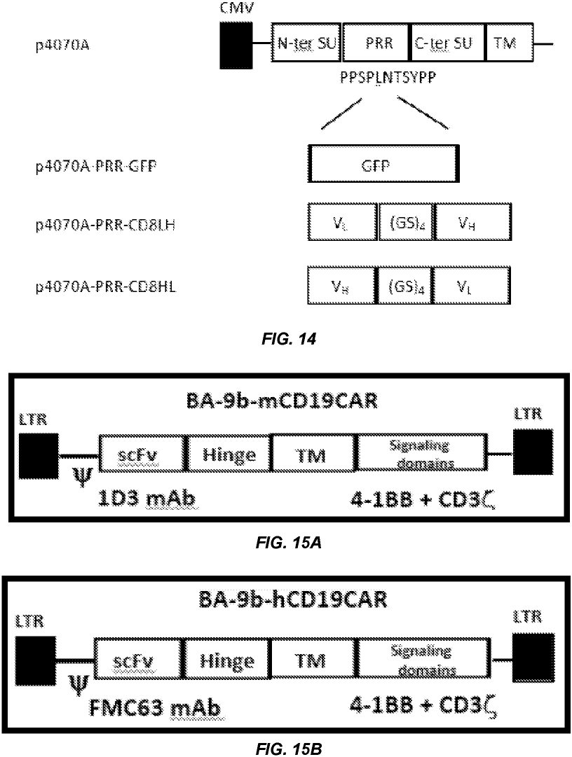

The amphotropic env can be engineered by modification of the proline rich region of 4070A env sequence. In FIG. 14, either GFP (as reporting marker to track along env expression) or scFV anti CD8 sequences (presented in both orientations) used to target viral transduction preferentially to CD8⁺ cells) are shown cloned into the L (Leucine)codon sequences of the proline rich region of the 4070A envelope sequence.

### Example 27

### CD34 Targeting

A. To achieve targeting to CD34+ cells, the Measles H protein can be used in conjunction with various targeting protein scaffold moieties to achieve targeting using single change fragment variable (scFv) monoclonal antibodies, designed ankyrin repeat proteins (DARPins) and bispecific antibodies that recognize 2 separate receptor epitopes. To minimize pre-existing immunity of individuals against the measles virus, the typical location of the H protein to engineer the targeting moiety is the H-Noose-Epitope of the Edmonston Measles strain. To blind H protein from recognizing its natural receptor, point mutations are engineered to destroy the recognition sequence. For targeting to CD34+ hematopoietic stem cells (HSCs), the CD34 targeting Anti-HPCA-1 monoclonal antibody is the preferred scFV moiety to use and engineer into the measles H protein sequence because receptor-mediated endocytosis is triggered on CD34+ hematopoietic cells after stimulation with the anti-HPCA-1 antibody. The use of the chimeric measles H protein with the anti-HPCA-1 scFV together with the use of the measles fusion (F) protein, will allow targeting and fusion MuLV pseudotyped viral vectors to CD34+ cells.

B. The Sindbis virus envelope proteins can also be used to pseudotype and target lentiviral and MuLV vectors to CD34+ cells by conferring CD34+ specificity at the level of cell entry. The ZZ domain of protein A has been incorporated into the Sindbis envelope, and vector transduction is achieved through antibody binding to specific antigens on the surface of the targeted cells. Several mutations, including deletion of amino acid 61-64 in the E3 protein and mutation of amino acids, K159A, E160A, and SLKQ68-71AAAA, in the E2 protein, have been generated in the Sindbis envelope glycoprotein to decrease the natural tropism of Sindbis and, consequently, its background infectivity, while maintaining high vector titers. Mutations of amino acids 226 and 227 in the E1 protein to S and G allow E1 to mediate fusion in the absence of cholesterol in the target membrane, thereby increasing both the tropism and infectivity of Sindbis pseudotyped vectors designated as envelope 2.2. The 2.2 vector has successfully targeted human leukocyte antigen (HLA) class I, CD4, CD19, CD20, CD45, CD146, P-glycoprotein of melanoma cells, and prostate stem cell antigen with CD34, CD133 and C-kit used for targeting human hematopoietic progenitor cells. Again the preferred antibody for targeting CD34+ cells is the anti-HPCA1, clone My10. However to increasing targeting, the use of bi-specific antibody that allows the use of both anti-HPCA1 along with anti C-kit+ moiety will enhance targeting.

C. Nipah chimeric envelope membrane bound G protein and F protein variants have been used for cell specific targeting with several advantages such as (1) shown not to have pre-existing immunity in the population, (2) have higher viral pseudotyped titers and (3) have a higher surface density.

### Example 28

### Producer Cell Lines Encoding Mouse or Human CD19 CAR Retroviral Vectors

The safety-modified pBA-9b retroviral vector is also similarly engineered to encode either mouse or human anti CD19CAR vector constructs in its basic construct form consisting of a scFV mAB-Hinge-TM domain region linked to a 4-1BB costimulatory signal domain with the anti-CD3z chimeric antigen receptor (CAR) genetic sequence (FIGS. 15A-B).

Retroviral non-clonal producer pools as well as clones are established (as referenced in FIG. 6) from clonal PCLs using a high multiplicity of transduction “high m.o.t.” approach with m.o.t.’s of >20 using a single or multiple back-to-back rounds of transductions. The multiplicity of transduction is defined as the number of infectious viral particles used per PCL cell for the production of VPCL non-clonal pools. Typically, a PCL culture is seeded at 1 × 10⁵ cells/well in a 6-well plate one day prior to transduction. The appropriate volumes of vector supernatants are then added to PCLs (in the presence of 8 µg/ml polybrene) corresponding to m.o.t.’s of 0.1, 0.5, 5, 25, and 125. After 20-24 h the vector supernatant is replaced with 2 ml of fresh media. To increase the m.o.t.’s, the transduction procedure can be repeated for a second day using the same volume of vector supernatant. Producer pools are grown to confluence and supernatants collected daily at 24, 48, and 72 h post-confluence to determine PCR titers and confer transfer of expression. Selected non-clonal pools are cloned using limited dilution seeding into 96 well plates that result in a single cell per well that are analyzed in several rounds of titer determination and transfer of expression assays as individual groups of clones expand.

### Example 29

### Modification to the pBA-9b-CD19CAR Sequence for Cell-Specific Detargeting

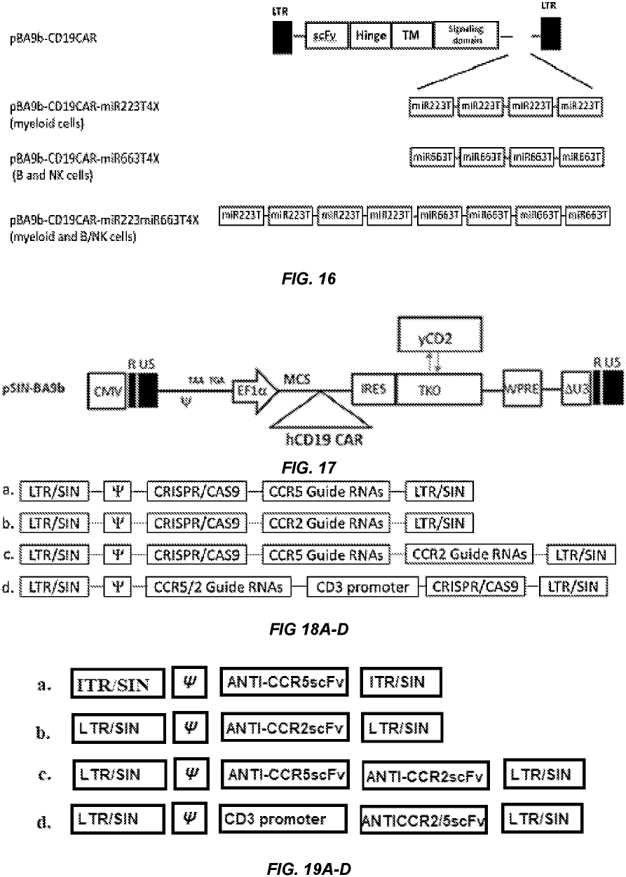

The basic pBA-9B-CD19 CAR sequence can be modified to also encode microRNA (miR) target sequences as an effective method of down regulating vector expression in cell type specific manner to increase the safety profile of the vector and prevent expression in non-intended cell types. FIG. 16 shows example miR target sequences used to impede expression in myeloid, B and NK cell types.

### Example 30

### Modification to pBA-9b to Create the Self-Inactivating (SIN) Vector pSIN-BA9b

To increase the safety of retroviral vectors, a SIN version of pBA-9b can engineered to eliminate activation of oncogenes due to LTR insertional mutagenesis. FIG. 17 shows an example design of pSIN-BA-9B with a hCD19 CAR gene cloned into the multiple cloning site.

### Example 31

### Determination of Key Properties of a Retroviral Vector Preparation

### A. Determination of Titer by Copy Number Quantitation by PCR

PCR titer analysis of vector samples is carried out. MLV-specific primers (5′-GCG-CCT-GCG-TCGGTA-CTA-G-3′ (SEQ ID NO:26), 5′-GAC-TCA-GGT-CGG-GCC-ACA-A-3′ (SEQ ID NO:27)) and probe (5′-AGT-TCG-GAA-CAC-CCG-GCC-GC-3′ (SEQ ID NO:28)) are used to amplify a 80-bp product. The amplification reaction is carried out in 50 µl with 200-400 µM dNTPs, 900 nM primers, and 100 nM probe oligonucleotide. The resulting fluorescence is detected and titer based on provector copy number expressed as transduction units/ml (TU/ml). Transduction units are defined as the provector copy number per genome equivalent relative to a known copy number standard, and represent a true reflection of vector integration units.

### B. Transfer of Expression Assay and Titer Determinations

This general titering assay utilizes HT-1080 target cells seeded one day prior to transduction at 3 × 10⁵ cells per well in a six-well plate (Corning Costar, NY). Polybrene (8 µg/ml) is added 2 h before transduction with serial dilutions of vector supernatants. After 20-24 h, the supernatant is replaced with 1-2 ml of fresh media. Cells are allowed to grow for an additional 24-48 h before supernatants or genomic DNA are assayed for transfer of expression of the gene product (TOE titer) or the number of provector copies present (PCR titer).

### C. β-Galactosidase Transfer Of Expression Titer Determination

The β-gal TOE titers are determined by two independent methods. The first method is a biochemical staining procedure using X-gal staining following a standard protocol. The second procedure is a chemiluminescent detection method utilizing the Galacto-light Plus Kit (Tropix, Inc., MA).

### D. Detection of Replication-Competent Retrovirus (RCR)

Two procedures are used to determine the presence or absence of RCR in the VPCL or the vector product, respectively. i) The first procedure tested post-production VPCLs. These cells are seeded into culture with an equal number of the replication permissive cell line M. dunni. VPCLs are seeded into flasks at a small scale (1 × 10⁷ cells) or roller bottles at a large scale (1 × 10⁸ cells). Cells are co-cultured for several passages and finally harvested. Cell free culture supernatant is tested using a marker rescue or PG4S + L-assay. An RCR producing cell line generated by infection of M. dunni cells with a hybrid murine leukemia virus served as a positive control for the cocultivation procedure. Naive M. dunni cells served as the negative control. ii) The second procedure tests the vector preparations directly. Unprocessed production harvest or purified bulk product is applied to M. dunni cells using 100 ml inoculation volume per roller bottle. After a brief inoculation period, 150 ml of additional media is added to the culture and cells are passaged four to five times before a portion of the culture supernatant is harvested, filtered, and assayed for RCR by a marker rescue or PG4S+ L- test. As per recent FDA guidance documents ([www.]FDA.gov), 300 ml of crude vector from clinical production lots are assayed for RCR. The M. dunni amplification (large scale) and PG4S + L-detection method used for product release has been validated for single unit RCR detection.

### Example 32

### Viral Production Using Multiple Cell Factories

The following example reviews cell culture methods needed to for growing adherent VPCL cells producing a murine leukemia virus (MuLV) for viral production. Production of a MuLV virus is used as an example, however this process can also be used for all virus described in this disclosure.

For this example, VPCL produced from the parental HT1080 cells (ATCC CCL-121) is described however the method can also be used for VPCLs produced from HEK 293T cells (CRL-1573), D-17 (ATCC CCL-183) and Cf2Th (ATCC CRL-1430) under the preferred conditions of 37° C. under 5% CO₂ conditions. For long term storage, VPCLs are cryopreserved under liquid nitrogen conditions, stored in cryoprotectant plastic vials containing 1 × 10⁷ cells frozen in a cryoprotectant cell culture media solution containing 10% DMSO, 50-90% fetal bovine serum in cell culture growth media solution. Upon thawing, cells are expanded by initially seeding into a T-75 flask with subsequent expansions into two T-175′s and then subsequently cultured to ten T-175 flasks with the following growth medium:

| Complete DMEM Medium components: | | Ratio |
|---|---|---|
| 1. | DMEM High Glucose, w/o phenol red & w/o glutamine | 500 mL |
| 2. | FBS Defined gamma-irradiated | 25 mL |
| 3. | GlutaMax (Gibco) | 5 mL |
| 4. | Non-essential amino acids (100x NEAA Stock) | 5 mL |

After reaching confluence, cells are harvested with TrpZean® (Sigma) and neutralized with the same growth medium using standard cell culture methods. The cells are seeded into three 10-layer Cell Stacks (Corning) at a preferred seeding density of 3.1 × 10^4 viable cells/cm² in the same medium previously described above, to produce the virus. Each Cell Stack contained 1.1 L of the growth medium. The Cell Stacks are incubated at 37° C. and 5% CO₂.

Two days after seeding, the Cell Stack cultures will approach or reach confluence. The medium in each culture is replaced with fresh medium. Two days later, the medium, containing produced virus, is harvested (Harvest #1), and the cultures re-fed with the same volume (1.1 L) of fresh medium. Ten hours later, a second harvest (Harvest #2) is conducted and the cell cultures re-fed with same volume (1.1 L) of fresh growth medium. Sixteen hours following 2^{nd} harvest, a 3^{rd} harvest (Harvest #3) is performed. The 3 harvests are then pooled for purification. Viral titers for the 3 harvests and the pool are listed in the following table.

| Harvest | Viral titer (# TU/mL) |
|---|---|
| Harvest #1 | 1.8 × 10^6 |
| Harvest #2 | 2.4 × 10^6 |
| Harvest #3 | 2.4 × 10^6 |
| Pool of 3 harvests | 2.1 × 10^6 |

### Example 33

### Viral Production by Another Cell Line With the Same Technique

The same culturing technique described in Example 32 can be used to produce any of the disclosed viruses, in the human cell line HEK 293 cells (ATTC# CRL-1573) and canine cell lines Cf2TH (ATCC# CRL-1430) or D-17 (ATCC # CCL-183). Three harvests, as described in Example 32, can be collected and pooled. The viral titer in the harvest pool using Cf2TH cells can be 4.9 × 10^6 TU/mL.

### Example 34

### Pharmacological Inhibition of HSPC-Niche Interactions Leading to Mobilization

Migration across the bone marrow endothelium, i.e. transendothelial migration, is a key step in the regulation of progenitor cell mobilization. A central mechanism that regulates HSPC-directed migration from blood to bone marrow and retention in the bone marrow niches involves activation of the CXCR4 receptor on HSPCs by chemokine CXCL12 (also known as Stromal cell-Derived Factor-1 (SDF-1) (Nagasawa et al. 1996; Oberlin et al. 1996) expressed on different subsets of stromal cells including reticular Nestin+ mesenchymal stem and progenitor cells (MSPCs) (Mendez-Ferrer et al. 2010), human reticular CD146+ MSPCs (Sacchetti et al. 2007) and perivascular reticular leptin receptor+ cells (Ding et al. 2012) .

CXCL12 secreted by these cells and adsorbed to the extracellular matrix inducing a CXCL12 gradient and induction of HSPC adhesion to the bone marrow niches (Sugiyama et al. 2006). Interference with CXCL12/CXCR4 interaction, such as by conditional deletion of CXCR4 or CXCL12 in mice, resulted in a reduced retention of HSPCs in the bone marrow and in dramatically increased migration of HSPC numbers into the peripheral blood and into spleen (Tzeng et al. 2011). CXCL12 (SDF-1) itself activates cell-surface integrins VLA-4, VLA-5, and LFA-1 (Peled et al. 2000). Activation of CD34(+) cells with CXCL12 (SDF-1) led to firm adhesion and transendothelial migration, which was dependent on LFA-⅟ICAM-1 and VLA-4/VCAM-1 interactions, and furthermore, CXCL12 (SDF-1)-induced polarization and extravasation of CD34(+) / CXCR4(+) HSPC through the extracellular matrix underlining the endothelium was dependent on both VLA-4 and VLA-5 (Peled et al. 2000). It has further been reported that CXCL12 (SDF-1) also activates the adhesion molecule CD44 and thereby rapidly and potently stimulates HSPC adhesion to immobilized hyaluronic acid and homing of HSPC to bone marrow, which could be blocked by anti-CD44 monoclonal antibodies or by soluble hyaluronic acid, and significantly impaired after intravenous injection of hyaluronidase (Avigdor et al. 2004). Thus, inhibition of CXCL12 (SDF-1)/CXCR4 interaction has downstream effects on multiple HSPC-stromal adhesion interactions.

AMD3100 (Plerixafor) is a synthetic organic molecule of the bicyclam class, originally developed as an anti-HIV drug (De Clercq 2019). By antagonizing the CXCR4 receptor, thus interfering with the CXCR4 / CXCL12 (SDF-1) interaction which tethers stem cells to the bone marrow stroma, AMD3100 was found to rapidly mobilize HSPCs in various animal models (Broxmeyer et al. 2005). In 2008, AMD3100/Plerixafor (trade name Mozobil) was approved in the United States for use in combination with G-CSF to mobilize HSPCs to the peripheral blood for collection and subsequent autologous transplantation in patients with Non-Hodgkin’s lymphoma or multiple myeloma, and is used as a supportive measure in cases where G-CSF-mediated HSPC mobilization fails to induce sufficient numbers of HSPCs (De Clercq 2019). The rapid mobilization by AMD3100 does not only regulate CXCL12 levels, but also induces activation of proteases such as matrix metalloprotease-9 (MMP-9) and urokinase-type plasminogen activator (uPA) (Dar et al. 2011). Activated bone marrow stromal and endothelial cells also play a role in secreting CXCL12 (SDF-1) into the circulation upon stimulation with AMD3100 (Dar et al. 2011).

Moreover, AMD3100 has served as the model for investigation of other drug compounds that similarly target the CXCR4 receptor and could be potentially useful as stem cell-mobilizing agents, such as KRH-1636 and CX0714 (De Clercq 2019). T-140 (4F-benzoyl-TN14003) is another CXCR4 inhibitor that mobilizes HSPCs and also erythroblasts in murine models and displays synergy with G-CSF (Abraham et al. 2007).

Firategrast, an α4β1 and α4β7 integrin inhibitor, has been shown to disrupt HSPC retention in the postnatal hematopoietic niche and to synergistically interact with the CXCR4 inhibitor AMD3100 (Kim et al. 2016).

Vedolizumab, a humanized monoclonal antibody against the α4β7 integrin, is available and has been tested in clinical trials for Crohn’s disease and ulcerative colitis, and more recently for prevention of graft-vs.-host disease after allogeneic HSPC transplantation (Chen et al. 2019). Reduction in plasma CXCL12 levels, accompanied by reduced HSPC egress, has been observed in mice treated with vedolizumab.

BOP (N-(benzenesulfonyl)-L-prolyl-L-O-(1-pyrrolidinylcarbonyl)tyrosine) is a chemical compound which also causes targeted interference with integrins α9β1/α4β1. It has been reported that a single dose of this small molecule antagonist rapidly mobilizes long-term multi-lineage reconstituting HSPC, and also enhanced AMD3100-induced HSPC mobilization (Cao et al. 2016).

Rac inhibitors: The Rac small GTPases expressed in hematopoietic progenitors and HSPCs have been found to be intricately involved in HSPC mobilization, and application of a specific inhibitor of Rac activity (but not Cdc42 or RhoA activity) may be used for rapid HSPC mobilization (Cancelas et al. 2005). Recently, it was shown that Rac1 activation leads to reversible conformational change in human CXCR4 that potentiates CXCL12 / CXCR4 signaling, implying reciprocal cross-talk between these signaling pathways (Zoughlami et al. 2012).

### Mobilization by Hematopoietic Growth Factor

Hematopoietic growth factors, especially granulocyte colony stimulating factor (G-CSF, filgrastim, lenograstim) and granulocyte-macrophage colony stimulating factor (GM-CSF, molgramostim, sargramostim) have been shown to be effective at mobilizing HSPC into the peripheral blood, to levels up to 60-fold over baseline using mobilization with hematopoietic growth factors alone (Peters et al. 1993; Gazitt 2002).

This approach is advantageous when mobilization can be planned prospectively, and can be performed without the associated morbidity of chemotherapy. In addition, it is possible for patients to receive administration of hematopoietic growth factors for mobilization at home, and then undergo further gene transfer procedures as outpatients.

G-CSF: Endothelial cells have been shown to be the main constitutive source of infection-induced expression of G-CSF in bone marrow (Boettcher et al. 2014). It is therefore likely that these signals are related to or part of mobilization of HSPCs. Of note, neutrophils permeabilize the sinusoid endothelial barrier in the bone marrow, as shown by transmission electron microscopy studies (Lee et al. 2009). However, disruption of the bone marrow endothelial barrier is prominent during repetitive G-CSF stimulations, indicating that the endothelial cells do function as a target of G-CSF during HSPC mobilization (Szumilas et al. 2005).

In osteoblasts, SCF/c-kit ligand, IL-7, and VCAM-1 and, as a counter-regulator of HSPC maintenance, osteopontin are all selectively down-regulated during mobilization by G-CSF treatment or after β3 adrenoreceptor activation (Mendez-Ferrer, Battista, and Frenette 2010). Since G-SCF stimulates β-adenergic sympathetic nervous activity, this provides evidence for a signaling chain inducing HSPC mobilization.

Furthermore, G-CSF treatment was shown to induce a robust expansion of neutrophils within the bone marrow, and they are the first cells to egress from the bone marrow during mobilization after G-CSF administration (Day and Link 2012). All three classical types of HSPC mobilization by external stimuli, i.e. G-CSF, chemotherapy and chemokines, are disrupted if the G-CSF receptor is absent in neutrophilic granulocytes, but not if it is absent in HSPC despite normal hematopoiesis (Liu, Poursine-Laurent, and Link 2000). In particular, G-CSF receptor has been shown to be required for cyclophosphamide- or IL-8-induced, but not FLT3L-induced mobilization (Liu, Poursine-Laurent, and Link 2000). Serine proteases and metalloproteinases released by neutrophilic granulocytes have been shown to cleave VCAM-1, c-kit, SCF, and CXCL12 (SDF-1) from stromal niche cells or HSPCs and include neutrophil elastase, cathepsin G and MMP-9 (Levesque et al. 2002; Heissig et al. 2002). These proteases are released by neutrophils as a last step during the mobilization process after G-CSF, and during mobilization with chemokines or chemotherapy, leading to rapid egress of HSPCs and progenitors into the circulation. Granulocyte colony-stimulating factor receptor (G-CSFR) has been also been reported to signal in monocytes to mobilize HSPCs into the blood stream, by suppressing the supportive role of osteoblasts and disrupting the CXCL12 / CXCR4 axis (Christopher et al. 2011).

It should be noted that diabetic mice show increased HSPC retention in the bone marrow and poor HSPC mobilization upon G-CSF (Ferraro et al. 2011). Down-modulation of CXCL12 (SDF-1) with G-SCF is absent in these animals. The defect could be salvaged by application of AMD-3100 and shows that mobilization of HSPCs may be influenced by diseases outside the hematopoietic system. Absence of c-Met signaling in HSPCs results in strong impairment of the HSPC egress from the bone marrow to blood, whereby blocking of CXCR4 prevented G-CSF-induced c-Met activation and HSPC mobilization (Tesio et al. 2011; Petit et al. 2002). Epidermal growth factor has also been shown to inhibit G-CSF-induced HSPC mobilization (Ryan et al. 2010).

Myelopoietin: Myelopoietin (MPO), a multifunctional agonist of interleukin-3 (IL-3) and G-CSF receptors, has also been reported to be an effective and efficient mobilizer of hematopoietic colony-forming cells (CFC) and CD34+ HSPC relative to control cytokines in normal nonhuman primates (MacVittie et al. 1999).

VEGF: Growth factor-stimulated progenitor cells produce large quantities of cytokines (e.g. vascular endothelial growth factor, VEGF) which may act on endothelial cells to modify their growth, motility, permeability, and fenestration. Increased vascular leakage as well as 2- to 3-fold increases in HSC numbers in the blood occurred within 15 min of an intravenous administration of rhVEGF164 or histamine (Smith-Berdan et al. 2015). VEGF may also be specifically involved in the mobilization and homing of hematopoietic progenitor cells as indicated by murine models in which hematopoiesis did not develop in the absence of VEGF or VEGF-receptors (Shalaby et al. 1995). Notably, however, administration of VEGF followed by treatment of mice with AMD3100 resulted in mobilization of both endothelial progenitor cells and stromal progenitor cells, but suppressed HSPC mobilization (Pitchford et al. 2009) .

SCF: Cytokine receptors such as c-kit, the receptor for the cytokine kit-ligand (stem cell factor, SCF) are also downregulated on circulating HSPC. Because membrane-bound cytokines such as kit-ligand (SCF) are expressed on bone marrow stromal and endothelial cells, c-kit may also act as an adhesion molecule and play a role in progenitor cell mobilization and homing. Downregulation of CXCL12 (SDF-1) in the bone marrow was also observed upon administration of SCF and FLT3-L (Christopher et al. 2009). As with adventitial reticular cells, osteoblastic cells secrete β-AR agonists to down-regulate expression of CXCL12, VCAM-1, and SCF (Katayama et al. 2006; Mendez-Ferrer, Battista, and Frenette 2010).

Complement factors: Monocytic cells are involved through the complement cascade, which is activated by radio- and chemotherapy, thereby liberating the complement factors C3 (C3a, desArgC3a) and C5 (C5a and desArgC5a) cleavage fragments, which act as anaphylatoxins (Ratajczak et al. 2013). Complement factor 3 (C3) knockout mice, under steady-state conditions, are hematologically normal but display a significant delay in hematopoietic recovery subsequent to irradiation or transplantation of wild-type HSPCs, and C3 complement factors increase responsiveness of HSPCs to CXCR4 / CXCL12 axis (Ratajczak et al. 2013), also suggesting an involvement of the classical complement activation pathway in HSPC mobilization. Moreover, complement factor 5(C5)-deficient mice showed impaired mobilization of HSPCs (Ratajczak et al. 2013).

Sphingolipids and Nucleotides: In addition to CXCL12 (SDF-1) and its receptor CXCR4 a number of other chemoattractants, inducing migration of HSPCs are known. These include the sphingolipids sphingosine-1-phosphate (S1P) (Golan et al. 2012; Ratajczak et al. 2014) that couple to G protein-coupled sphingosine 1-phosphate receptor 1 (S1P1), and ceramide-1-phosphate (C1P) (Ratajczak et al. 2014). Moreover, extracellular nucleotides, such as adenosine triphosphate (ATP) or uridine triphosphate (UTP) (Rossi et al. 2007) and the divalent cation Ca2+ and its receptor (CaR) (Adams et al. 2006), and H+ (Krewson et al. 2020) regulate adhesion molecules on the surface of the HSPCs as well as on certain cells of the stem cell niches, such as endothelial cells. Whereas the cells of origin of these molecules are in many cases not clear as yet, their action includes the mediation of engraftment (after transplantation), adhesion (under steady state), and mobilization (after its induction).

S1P: S1P and the receptor S1P1 are suggested to regulate HSPC steady-state egress and mobilization from the bone marrow. S1P is produced by mature red blood cells and activated platelets, leading to micromolar S1P concentrations in blood, mostly bound to albumin and high-density lipoproteins (HDL) (Pappu et al. 2007; Liu et al. 2011). Since only low concentrations of S1P are detectable in solid tissues, a constant S1P concentration gradient between the bone marrow and blood important for the constant steady-state release of HSPCs has been suggested. This idea is supported by the finding that inhibition of S1P1 receptor with the specific inhibitor FTY720 decreases steady-state mobilization of HSPCs into the bloodstream (Golan et al. 2012; Liu et al. 2011). In line with this, over-expression of S1P1 receptor in HSPCs increased S1P-mediated migration, and a reduction of CXCR4 surface expression with a significant inhibition of in vitro CXCL12 (SDF-1) induced migration and reduced HSPC homing potential to the bone marrow (Ryser et al. 2008). Also, transiently increased S1P plasma concentrations are observed during mobilization with G-CSF or AMD3100 (Golan et al. 2012), probably due to increased hemolysis by activating the complement cascade and the membrane attack complex (Ratajczak et al. 2013; Ratajczak et al. 2014), suggesting that S1P is involved in release of HSPCs during mobilization (Golan et al. 2012). Increased bone marrow concentrations of S1P also induce CXCL12 (SDF-1) secretion from bone marrow Nestin+ MSCs, and a reduced release of HSPCs into the blood (Golan et al. 2012).

A second S1P receptor, S1PR3, expressed on HSPCs is suggested to be involved in the retention of HSPCs in the bone marrow niche. Inhibition or knockout of S1PR3 results in mobilization of HSPCs into blood circulation, whereby S1PR3 antagonism suppresses bone marrow and plasma CXCL12 (SDF-1) concentrations (Ogle et al. 2017). On the other hand, S1PR3 antagonism increases AMD3100 induced mobilization, indicating a synergy between S1PR3 and CXCR4 pathways.

C1P: After lethal irradiation, levels of S1P and C1P have been seen to increase in the bone marrow microenvironment; moreover, peripheral circulating HSPCs are exposed to relatively high levels of S1P and C1P present in the circulation. Both these mechanisms may desensitize responses to potential homing gradients of these bioactive lipids (Ratajczak et al. 2014).

ATP and UTP: 5-nucleotide triphosphates, particularly ATP and UTP, engage P2 nucleotide receptor-mediated regulation of proliferation, differentiation, cell death, and chemotaxis on hematopoietic cells including HSPCs (Lemoli et al. 2004). UTP is thought to represent an endogenous danger signal, which is rapidly released into the extracellular environment due to tissue injury and cell death. UTP and other nucleotides induce migration of leukocytes into the injured tissue, stimulate tissue recovery by induction of cell proliferation, and promote resolution of the immune response by activating anti-inflammatory pathways (Di Virgilio, Boeynaems, and Robson 2009). Preincubation of HSPCs with UTP significantly improved CXCR4-induced migration of HSPCs in vitro, whereas UTP itself only induces marginal chemotactic migration of HSPCs (Rossi et al. 2007). In addition, pre-treatment with UTP alone or in combination with CXCL12/SDF-1 significantly increases cell adhesion to fibronectin and UTP pre-treatment improves homing of human HSPCs to the bone marrow (Rossi et al. 2007). Involvement of UTP signaling in PBSC mobilization remains to be elucidated. Inhibition of CXCL12- and UTP-dependent chemotaxis by pertussis toxin suggests that Rho guanosine 5′-triphosphatase (GTPase) Rac2 and its effectors Rho GTPase-activated kinases 1 and 2 (ROCK½) are involved in UTP-regulated/CXCL12-dependent HSPC migration (Rossi et al. 2007).

Uridine Diphosphate-Glucose: Uridine diphosphate-glucose (UDP-glc) is released into extracellular fluids in response to stress. UDP-glc has been shown to mobilize long-term repopulating HSPCs, and co-administration of UDP-glc and G-CSF led to greater HSPC mobilization than G-CSF alone (Kook et al. 2013). In competitive repopulation experiments, HSPCs mobilized with UDP-glc plus G-CSF repopulated better than HSPCs mobilized with G-CSF alone. In comparison with G-CSF, UDP-glc-mobilized HSPCs exhibited a more lymphoid-biased differentiation capacity, indicating that UDP-glc mobilizes a functionally distinct subset of HSPCs (Kook et al. 2013). In contrast, inhibition experiments showed that reactive oxygen species (ROS) are mediators of UDP-glc-mediated HSPC mobilization. Application of the ROS inhibitor N-acetyl-L-cysteine (NAC) was able to significantly abrogate UDP-glc-induced HSPC mobilization. Kook et al. suggest that ROS induce enhancement in receptor activator of nuclear factor kappa-B ligand (RANKL) expression and a RANKL-induced osteoclast differentiation, leading to HSPC mobilization (Kook et al. 2013).

Ca2+ and CaR: HSPCs express the seven-transmembrane-spanning CaR which is strongly involved in retention and liberation of HSPCs in/from the stem cell niche. Newborn mice deficient in CaR revealed a reduced cellularity in the bone marrow and a lack of primitive HSPCs, whereas increased numbers of HSPCs were found in the circulation and spleen. The fetal liver of CaR-/- mice had normal numbers of HSPCs with normal proliferation, differentiation, and migrational capacity. But these HSPCs revealed an adhesion defect to collagen I, leading to a defective lodgment to the endosteal niche (Adams et al. 2006). On the other hand, pharmacological activation of CaR with Cinacalcet, a positive allosteric modulator of CaR, resulted in increased adhesion of HSPCs to collagen I and fibronectin, an increased in vitro migration towards CXCL12 (SDF-1) and an increased in vivo homing and lodgment to the endosteal niche (Lam, Cunningham, and Adams 2011). Ca2+ treatment increased transcription and expression of CXCR4 of bone marrow cells and SDF-1-mediated CXCR4 internalization, while Ca2+ influx inhibitors or blocking of CaR with antibodies inhibited Ca2+-induced CXCR4 expression, indicating that Ca2+-induced changes on HSPCs are partially modulated by an increased expression of CXCR4 (Wu et al. 2009).

Molecules Promoting Incorporation of CXCR4 into Membrane Lipid Rafts: Cationic antimicrobial peptides (CAMPs), Cathelicidin LL-37, β2-defensin, and C3a were found to highly improve responsiveness of HSPCs to picomolar levels of CXCL12 (SDF-1) (1-2 ng/ml), which reflect the physiological CXCL12 (SDF-1) concentrations in tissues, supporting the biological significance of this class of molecules in HSPCs (Wu et al. 2012). These molecules improve incorporation of the CXCR4 receptor into membrane lipid rafts (Ratajczak et al. 2013). Within membrane lipid rafts, several cellular signaling molecules are assembled, such as small guanine nucleotide triphosphatases (GTPases) Rac-1 and Rac-2, known to be important for the lodgment of HSPCs into niches or, when blocked, for mobilization (Cancelas et al. 2005). Co-localization of CXCR4 and Rac-1 in lipid rafts may improve GTP binding and activation of Rac-1.

Cathelicidin LL-37 is an antimicrobial peptide expressed by bone marrow stromal cells and is upregulated after bone marrow irradiation. It was shown that LL-37 enhances chemotactic migration and adhesiveness of HSPCs and that short-time pre-incubation of HSPCs with LL-37 prior to transplantation accelerates recovery of platelet and neutrophil counts in mice (Wu et al. 2012). Its effect on mobilization remains to be determined.

Prostaglandin E2: Murine and human HSPCs express prostaglandin E2 (PGE2) receptors. In murine transplantation experiments, short-term ex vivo exposure to PGE2 enhances homing and proliferation of HSPCs and increases the number of primitive, long-term repopulating cells, indicating that PGE2 supports self-renewal of HSPCs (North et al. 2007; Hoggatt et al. 2009). In contrast, differentiation of immature myeloid progenitor cells leading to colony-forming units-granulocyte macrophage (CFU-GM) and macrophage (CFU-M) is suppressed by PGE2 (Pelus et al. 1979), suggesting a differential regulation of the hematopoiesis by PGE2. Blocking of PGE2 production by nonsteroidal anti-inflammatory drugs (NSAID) such as indomethacin, aspirin, ibuprofen, and meloxicam doubled G-CSF-induced HSPC mobilization. Transplantation of G-CSF + NSAID-mobilized grafts was associated with a faster regeneration of posttransplant hematopoiesis (Hoggatt et al. 2013). The significance of PEG2 in the bone marrow niche is supported by the findings of i) an increased PGE2 production by irradiated bone marrow stromal cells, and ii) a C1P- and S1P-induced, increased expression of cyclooxygenase 2 in stromal cells, whereby C1P and S1P is released from lethally irradiated, damaged bone marrow cells, suggesting that conditioning for HSPC transplantation by lethal irradiation induces PGE2 production in bone marrow (Ratajczak et al. 2014).

PGE2 treatment increases cellular CXCR4 mRNA concentration and the expression of CXCR4 on the HSPC surface, leading to an enhanced migration to SDF-⅟CXCL12 in vitro and homing to bone marrow in vivo. Furthermore, PGE2 enhances HSPC survival due to an increased expression of survivin and reduction in intracellular active caspase-3 (Hoggatt et al. 2009). Another mechanism, which is important for PGE2-mediated modulation of HSPC retention and egress of HSPCs into the blood, is the regulation of osteopontin expression. It is suggested that blockade of PGE2 synthesis by NSAID lead to a reduction in osteopontin expression, which is responsible for the retention of HSPCs in the stem cell niche (Hoggatt et al. 2013). Also, osteopontin is a negative regulator of the size of the stem cell pool, and down-regulation or absence of osteopontin is associated with an increased number of primitive HSPCs (Stier et al. 2005). Thus, osteopontin expressed by stromal cells may be associated with a superior repopulating ability and long-term engraftment of G-CSF plus NSAID mobilized stem cell graft compared to grafts mobilized with G-CSF alone (Hoggatt et al. 2013).

EPI-X4, an Endogenous Human CXCR4 Antagonist: EPI-X4 is a 16 amino acid peptide isolated from human blood filtrate and plasma and generated from albumin under acidic conditions by aspartic proteases cathepsin D and E, which are released from immune cells during inflammatory processes (Zirafi et al. 2015). Neutrophils were shown to produce EPI-X4 from albumin (Zirafi et al. 2015). Since neutrophilic granulocytes in the bone marrow are strongly activated during HSPC mobilization with G-CSF, which leads to a highly proteolytic environment (Levesque et al. 2002), and since albumin is distributed throughout the extracellular space of the bone marrow, regulation of the CXCR4 / CXCL12 (SDF-1) axis by EPI-X4 production in the bone marrow is a possibility, which is in line with the idea that single intraperitoneal injection of EPI-X4 into mice resulted in marked mobilization of HSPCs, which engrafted lethally irradiated hosts (Zirafi et al. 2015).

GROβ, a truncated form of CXCR2 agonist: A rapid stem cell mobilization regimen utilizing an N-terminal 4-amino acid truncated form of the human CXCR2 chemokine agonist GROβ, in combination with the CXCR4 antagonist AMD3100, has recently been reported (Hoggatt et al. 2018). A single injection of both agents into mice resulted in stem cell mobilization peaking within 15 min that was equivalent in magnitude to a standard multi-day regimen of G-CSF. This rapid mobilization resulted from synergistic signaling on neutrophils, resulting in enhanced MMP-9 release, and it was found that genetic polymorphisms in MMP-9 can alter the activity of this regimen (Hoggatt et al. 2018). Similar results were previously reported using N-terminal truncated GROβ (variously designated as SB-251353 (King et al. 2001) or SK&F 107647 (King et al. 2000)) alone or in combination with G-CSF in non-human primates (King et al. 2001).

Sildenafil: A factor which is likely dependent on monocytes and which regulates HSPC mobilization is nitric oxide. Mobilization studies in inducible nitric oxide synthase (iNOS) -/mice indicated that iNOS is a negative regulator of hematopoietic cell migration and prevents egress of HSPCs into peripheral blood during mobilization (Adamiak et al. 2017). Sildenafil citrate (Viagra), a phosphodiesterase type 5 (PDE5) inhibitor, blocks degradation of cyclic GMP in the smooth muscle cells lining blood vessels, resulting in vasodilation. This inhibition is immediate, with a peak activity 2 h after oral administration of the drug (Andersson 2018). A recent study found that a rapid, 2-h regimen of a single oral dose of sildenafil citrate, combined with a single injection of CXCR4 antagonist AMD3100, leads to efficient HSC mobilization at levels rivaling the standard-of-care 5-day regimen of G-CSF (Filgrastim/Neupogen) (Smith-Berdan et al. 2019).

### Choice of Mobilization Regimen Can Affect HSPC Phenotype

Analysis of antigen expression has shown that there is phenotypical variance between mobilized and steady-state CD34+ cells and the choice of mobilizing hematopoietic growth factor, with or without chemotherapy, may impact on harvest subpopulations of CD34+ cells. The choice of mobilization regimen may therefore have an impact on the hematopoietic reconstitution of specific cell lineages from mobilized HSPC.

Cyclophosphamide-mobilized CD34+cells have few pre-B lymphocytes, as determined by low CD19 expression, together with low CD71 expression, suggesting a lack of active proliferation, and variation in the incidence of the subpopulations between different patients.

Conversely, mobilization with both chemotherapy and G-CSF shows heterogeneity of CD33 and CD71 co-expression in different patients, and a high level of CD71 expression.

Evidence suggesting that long-term platelet recovery is more accurately predicted by the number of transplanted primitive CD34+ CD33- cells, rather than the heterogenic CD34+ cell population from which it is derived, is beginning to accumulate.

The lack of expression of the CD38 antigen (CD38-), a characteristic of early human progenitor cells, may be influenced by the mobilization regimen employed. G-CSF-mobilized PBPC contain a significantly greater proportion of the primitive progenitors (only 88% of cells expressing CD38) than those mobilized by chemotherapy plus G-CSF (97% express CD38), chemotherapy plus GM-CSF (96.4% express CD38) or high-dose chemotherapy alone (99.1% express CD38).

Similarly, specific mobilization regimens have been described that may predict for increased harvest yields of long-term culture-initiating cells (LTCIC).

### Example 35

### Chemotherapeutic Drug Regimens for Mobilization

The number of circulating HSPC can be significantly increased during the recovery period after myelosuppressive chemotherapy, as compared to steady-state levels.

For example, a single dose of 4 g/m² cyclophosphamide can result in up to 25-fold increase in the number of CFU-GM.

Similarly, CD34+ cell yields on the order of 3.62 × 10⁶ cells/kg can be achieved following cyclophosphamide doses of 4-7 g/m2) .

High-dose etoposide (2 g/m²) may also be used as a safe and effective method to mobilize HSPC.

Combination of high-dose cyclophosphamide with etoposide (600 mg/m²) leads to mobilization of HSPC that are superior in CFU-GM and CD34+ cell content than those achieved with individual chemotherapeutic agents.

The number of mobilized colony-forming cells appears to be highest following the first cycle of an induction chemotherapy regimen, with a reduction in numbers after each subsequent cycle.

However, analysis of these mobilized cells shows a greater proportion of CD34+ cells following the fourth chemotherapy cycle than following the first cycle.

Chemotherapy-induced mobilization may avoid delay in the administration of effective anti-cancer treatment and allows in vivo purging in addition to HSPC mobilization.

### Example 36

### Hematopoietic Growth Factor Regimens for Mobilization

G-CSF: G-CSF alone can effectively mobilize HSPC from the bone marrow into the peripheral blood.

A dose of filgrastim 10 µg/kg/day is usually adequate, but some studies have used 16 µg/kg/day.

The capacity of G-CSF to increase the circulating number of progenitor cells was first noted by Durhsen et al34, and further studies have shown that G-CSF mobilizes progenitor cells from the bone marrow into peripheral blood in patients with various malignant diseases.

Sheridan et al. investigated the use of G-CSF (12 µg/kg/day for 6 days) alone to mobilize PBPC in 17 patients with non-myeloid malignant disorders of poor prognosis. A mean total of 33 × 10⁴ CFU-GM/kg were harvested. Similarly, G-CSF (10 µg/kg/day) adequately mobilized PBPC in 34 patients with Hodgkin’s disease or NHL, allowing the harvest of a median 32.6 × 10⁴ CFU-GM cells/kg and 2.8 × 10⁶ CD34+ cells/kg. De Arriba et al. reported a mean yield of 0.77 × 10⁶ CD34+ cells/kg and 1.42 × 10⁶ CD34+ cells/kg after the first and second leukapheresis, respectively, in 10 breast cancer patients following mobilization with G-CSF alone (0.84 ± 0.1 µg/kg/day) .

### G-CSF Dose-Response Effect

A mobilization dose-response effect is apparent in healthy adults and patients. An increase in G-CSF dose from 5 µg/kg/day to 10 µg/kg/day led to an increase in CD34+ content of peripheral blood from sevenfold to 28-fold over baseline.

Similarly, increasing the dose of G-CSF from 10 µg/kg/day to 24 µg/kg/day significantly improves the CD34+ HSPC yield (11.32 × 10⁷/kg cells versus 48.25 × 10⁷/kg cells).

The characterization of the PBPC mobilized may also be affected by the size of the administered G-CSF dose. Increased mobilization of less mature progenitors into the circulation (i.e. mixed colony-forming cells, CD34+ CD33- cells and CD34+ HLA-DR-cells) may occur when the dose of G-CSF is raised from 100 to 200 µg/m².

Practical example (G-CSF): Based on current dosage recommendations for HSPC mobilization,10 µg/kg/day of filgrastim or lenograstim are administered as de novo mobilization, or 5 g/kg/day for filgrastim or lenograstim when given in combination with chemotherapy (Amgen; Chugai Pharma UK Ltd/Rhône-Poulenc Rorer), for at least 4 days. Virus vector infusion can commence on day 5 and continue for 3 consecutive days.

GM-CSF: GM-CSF is also effective for mobilizing HSPC, with doses of 4-64 µg/kg/day by continuous intravenous infusion for up to 7 days in cancer patients resulting in 4- to 18-fold increased HSPC in the peripheral blood.

HSPC mobilization can be achieved using GM-CSF alone, although it should be noted that, while the feasibility of using GM-CSF for progenitor cell mobilization has been confirmed, there is no evidence of superiority over mobilization with G-CSF alone.

In a double-blind study by Hohaus et al., 26 patients with relapsed Hodgkin’s disease received chemotherapy and were randomized to receive either G-CSF (5 µg/kg/day, N = 12) or GM-CSF (5 µg/kg/day, N = 14) beginning on the first day following chemotherapy. No significant difference was observed in the median yield of CD34+ cells (7.6 × 10⁶ versus 5.6 × 10⁶ CD34+ cells/kg for G-CSF and GM-CSF, respectively).

Villeval et al. treated 37 patients with 0.3-30 µg/kg/day subcutaneous or 0.3-20 µg/kg/day short intravenous infusion and found that the levels of blood GM-CFC was significantly elevated after 4 to 5 days treatment and additionally there was a clear dose-response effect.

The CFU-GM content of HSPC populations mobilized by GM-CSF appears to be dose responsive with a 250 µg/m2 dose producing a superior yield to 125 µg/m2.

It should be noted, however, that while the administration of G-CSF (5 µg/kg/day) or GM-CSF (5 µg/kg/day) alone for 4 days produced a dramatic increase in the harvest of CFU-GM over baseline (35.6 and 33.7-fold, respectively), the administration of G-CSF (5 µg/kg/day) for a further 5 days to patients who had already received 7 days of GM-CSF (5 µg/kg/day) resulted in an 80-fold increase in HSPC over baseline.

However, in healthy subjects, mobilization with a combination of G-CSF (5 µg/kg/day) plus GM-CSF (5 µg/kg/day) did not produce a significant increase in harvest yield when compared with mobilization with G-CSF (10 µg/kg/day) alone (mean, 101 × 10⁶ CD34+ cells/kg versus 119 × 10⁶ CD34+ cells/kg, respectively).

### Example 37

### Chemotherapy Plus Hematopoietic Growth Factor Regimen For Mobilization

The objective of the introduction of a hematopoietic growth factor into a chemotherapy-based mobilization regimen is to enhance HSPC yield while supporting early anti-cancer treatment. The addition of a hematopoietic growth factor after chemotherapy significantly increases the number of HSPC mobilized in comparison with chemotherapy alone.

As noted, the inclusion of chemotherapy in the mobilization regimen may be important for the prompt initiation of the treatment of some tumors. However, when this is not required, the adverse effects of chemotherapy should be carefully considered, and a hematopoietic growth factor-only mobilization regimen may be more appropriate. Also, repeated chemotherapy plus G-CSF may lead to induction of fewer long-term culture initiating cells (LTC-IC) than following mobilization with G-CSF alone.

A wide variety of combination regimens have been employed, and while there is no consensus recommendation regarding the optimal dosing or schedule, the combination of cyclophosphamide at 4-7 g/m² plus G-CSF at 5 µg/kg/day is commonly used.

### Regimes in Clinic

G-CSF plus cyclophosphamide: The combination of G-CSF with high-dose cyclophosphamide (4-7 g/m²) has been shown to improve the yield of CD34+ HSPC levels in the blood.

Higher doses of cyclophosphamide may be used to improve the yield of HSPC. For example, patients with multiple myeloma, can be mobilized with high-dose cyclophosphamide 7 g/m² plus G-CSF (300 µg/day), resulting in significantly higher yields of >2.5 × 10⁶ CD34+ cells/kg, than could be obtained with cyclophosphamide 4 g/m² plus G-CSF.

G-CSF plus etoposide: Etoposide has been shown to be effective in the treatment of NHL, Hodgkin’s disease and, to a lesser extent, breast cancer. Inclusion in the mobilization regimen of patients with such malignancies is logical to provide treatment during the mobilization phase.

For example, patients with breast cancer, NHL or Hodgkin’s disease had HSPC mobilized with high-dose etoposide (2 g/m²) by continuous intravenous infusion over 24 hours plus G-CSF (5 µg/kg/day) beginning 48 hours after the completion of the etoposide infusion.

The mobilization regimen was effective, as evidenced by median yields of 24 × 10⁶ CD34+ cells/kg (range: 9.5-27.7 × 10⁶/kg), 28.0 × 10⁶ CD34+ cells/kg (range: 1.7-81.8 × 10⁶/kg), 22.7 × 10⁶ CD34+ cells/kg (range: 2.7-79.3 × 10⁶/kg) mobilized in the patients with Hodgkin’s disease, NHL, and breast cancer, respectively, measured starting from the first day that the white blood cell count recovered to 1 × 10⁹/L (median 10 days post-etoposide, range 7-16 days).

G-CSF plus cyclophosphamide plus etoposide: HSPC mobilization can also be achieved following high-dose cyclophosphamide (4 g/m²) plus etoposide (600 mg/m²), with or without G-CSF, in patients with breast cancer, myeloma and other malignancies. The inclusion of G-CSF in the mobilization regimen led to a yield with nearly 5-fold more CD34+ cells than following mobilization with cyclophosphamide and etoposide alone.

G-CSF plus combination chemotherapy: Combination chemotherapy with 5-fluorouracil (500 mg/m²), epirubicin (120 mg/m²) and cyclophosphamide (500 mg/m²) intravenously on day 1, followed by G-CSF (10 µg/kg/day) from day 2, resulted in a median 17.7 × 10⁶ (range: 9.4-50.6 × 10⁶) CD34+ cells/kg in patients with breast cancer.

Similarly, the further addition of other chemotherapeutic drugs, including but not restricted to, e.g., paclitaxel, to regimens combining cyclophosphamide plus G-CSF may produce further improvement in mobilization.

### Example 38

### Clinical HIV Prevention

Previously, two patients (referred to as “Berlin” and “London” patients (R.K. Gupta et al. Nature 2015) who were HIV-positive and received allogeneic bone marrow transplantations from CCR5Δ32 homozygous donors showed remission and complete absence of detectable viremia post-HSC transplantation. Therefore, knocking out CCR2 and/or CCR5 from HSCs is a viable way to prevent HIV infection or introduce remission in patients already viremic with HIV. A non-replicating retroviral vector (pBA-9b) is engineered to contain one of several mechanisms to reduce or knockout CCR5 or CCR2 gene expression from HSCs. In one example, the vector encodes CRISPR/CAS9 and guide RNAs to knockout CCR5 (FIG. 18A). In another example, the vector encodes CRISPR/CAS9 and guide RNAs to knockout CCR2 (FIG. 18B). In additional examples, the vector encodes CRISPR/CAS9 and guide RNAs to knockout both CCR5 and CCR2 (FIG. 18C). In further examples the vector encodes a single chain antibodies, including camelid and shark antibodies or other protein binding molecules such as darpins, affimers, hikamers and the like U.H.Wiedle et al. Cancer Genomics & Proteomics 10: 155-168, 2013; K.Skrlec et al. Trends inBiotechnology,33:408-418, 2015), which are produced within the target cell and block the CCR5 protein’s activity.

Further embodiments include the addition of lineage specific promoters so that CCR5 and CCR2 are knocked out in the desired T cell population. In this example, CRISPR/CAS9 is driven by a CD3 promoter so that expression of CRISPR/CAS9 occurs in the T cell lineage from post-HSC transduction by vector and subsequent HSC maturation (FIG. 18D). Additional T cell promoters may be substituted including CD4 and CD8 promoters.

In further embodiment the vector encodes a single chain antibodies, including camelid and shark antibodies or other protein binding molecules such as darpins, affimers, hikamers and the like (U.H. Wiedle et al. Cancer Genomics & Proteomics 10: 155-168, 2013; K.Skrlec et al. Trends in Biotechnology, 33:408-418, 2015), which are produced within the target cell and block the CCR5 and/or CCR2 protein’s activity (FIGS. 19A-D).

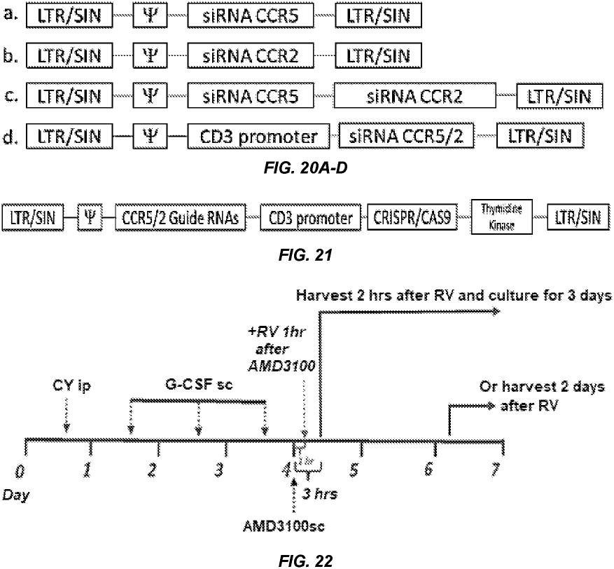

In yet another embodiment, CRISP/CAS 9 is replaced with shRNA or microRNA or silencing RNA (all referred to as siRNA) that knockdown expression of CCR5 and or CCR2 using the same configuration as for the CRISPR/CAS9 system (FIGS. 20A-D).

In addition to prevention and inducing remission of HIV infected patients, the vectors described in FIG. 21 is used to treat patients diagnosed with multiple sclerosis. In this embodiment, CRISPR/CAS9 or siRNAs are used to remove or reduce CCR5 and CCR2 expression from T cells using the HSC linage specific CD3 promoter. In addition, the vector contains a cellular kill switch, thymidine kinase (TK) that allows for the termination of treatment or to stop acute inflammatory episodes whereby patients are given TK prodrug that is converted into a toxic chemical that kills the TK expressing cells (FIG. 21). Human examples:

1. A patient viremic with HIV is taken off antivirals that are known or suspected to inhibit retroviral vectors for 2-10 days, then administered a dose of E6, E7, E8 E9, E10, E11 or E12 of a clinically acceptable (GMP) preparation of the retroviral vector that contains transgenes for CRISPR/CAS9 and guide RNAs to knockout CCR5. Vector infusion is complimented with an appropriate adjuvant that mobilizes HSC from the relatively inaccessible bone marrow to the accessible periphery, as described in the examples herein. Post-engraftment the patient’s T cells show loss of CCR5 expression over time and subsequent reduced levels of HIV DNA positive T cells in the positive cells in the blood and increased levels of transduced HIV-free T cells. After productive engraftment of CCR5 knocked out in the patients HSCs, and subsequent T cell renewal, the majority of patients T cells are converted to CCR5 negative and are resistant to HIV infection within 1 -3 months. Over time, HIV is cleared from the patient’s body and the patients goes into long term remission with long-term resistance to HIV.

2. A multiple sclerosis patient at the early, inflammatory phase of the disease undergoes dosing with a GMP preparation of retroviral vector with a titer of E8 TU/ml or greater on PC3 Cells designed to silence CCR⅖ in HSC and descendant T cells post-vector transduction. Dosing parameters are the same or similar to those used for the HIV patient with pretreatment by an appropriate, mobilizing agent. The loss of CCR2 and CCR5 prevents T cells from migrating to MS-related inflammation and prevents damage to tissues. In addition, ganciclovir is given to the MS patient during acute phase of MS and leading to depletion of marked T cells and reduction in MRI-measured lesions in the patient. The patient then undergoes repeat vector/mobilizing agent therapy to restore the HSC population. The reduction in MRI-detectable lesion is associated with the loss of CCR⅖ in the patient’s T cells.

3. A pediatric Adenosine DeAminase (ADA) deficient Severe Combined Immuno-Deficient (SCID) patient is dosed with a GMP preparation of retroviral vector with a titer of E8 TU/ml or greater on PC3 Cells that leads to expression of human ADA in transduced cells. Doses are between E6 to E12 total TU’s and are preceded by appropriate dosing with an HSC mobilizing agent. The recovery to normal or pseudo-normal immune function is as described by A.Aiuti et al. NEJM 2009 after conventional transplantation of ex vivo transduced autologous stem cells into pediatric ADA SCID patients.

### Example 40

### Detailed Human Dosing Protocol for Efficient HSC Mobilization Using Plerixafor Without or With G-CSF

Plerixafor can be used to specifically mobilize CD34+ HSPCs, either used alone or as an adjunct to G-CSF. The doses used are 160 µg/kg × 1 on day 5 for plerixafor, and 10 µg/kg on days 0, 1, 2, 3 and 4 for G-CSF, or 240 µg/kg for plerixafor if used alone. A single dose of plerixafor at 240 µg/kg (subcutaneously) may provide a more rapid and possibly less toxic and cumbersome alternative to traditional G-CSF based mobilization. However, the combination of G-CSF (10 µg/kg subcutaneously daily for up to eight days, together with plerixafor, beginning on the evening of day 4 and continuing daily for up to four days, subcutaneously at a (daily) dose of 240 µg/kg, has been approved by FDA and recommended for autologous stem cell mobilization and transplantation for patients with Non-Hodgkin’s lymphoma or multiple myeloma.

The conventional dose of plerixafor is 240 µg/kg, but this dose could be safely increased (in healthy individuals) to 480 µg/kg. After subcutaneous injection of 240 µg/kg plerixafor, CD34+ HSPC counts in the blood elevate from 1-2 cells/uL to a peak of approximately 25 cells/µL within 8-10 hours, and gradually diminish but are still at approximately 10 cells/µL by 24 hours post-injection. If a subcutaneous injection of 480 µg/kg plerixafor is administered, CD34+ HSPC counts in the blood elevate from 1-2 cells/uL to a peak of approximately 30 cells/µL within 8-10 hours, and gradually diminish but are still at approximately 20 cells/µL by 24 hours post-injection.

### Example 41

### Detailed Mouse Protocol for Mobilization of HSC Using Sildenafil (Viagra) Plus Plerixafor

Viagra was administered via oral gavage (OG) (3 mg/kg) to mice, once, 1 h before a single subcutaneous (SQ) injection of AMD3100 (2.5 mg/kg). Control mice received 5-day G-CSF treatment, administered once daily (250 mg/kg). Blood was collected by perfusion 1 h after AMD3100 or 24 h after G-CSF and analyzed by flow cytometry and multilineage reconstitution of lethally irradiated recipients.

Five-day, multidose G-CSF mobilization was not significantly better than the 2-h Viagra + AMD3100 mobilization protocol. Higher doses of Viagra (10 and 30 mg/kg) also improved AMD3100-mediated HSC mobilization, but were not more effective than 3 mg/kg. Furthermore, a 3-day oral Viagra regimen combined with a single AMD3100 injection led to significantly more HSCs in the bloodstream than AMD3100 alone. Compared with control mice, the numbers of phenotypic HSCs increased 3-, 7.5-, and 8.5-fold with AMD3100 alone, AMD3100 plus a single Viagra dose, and AMD3100 plus 3 days of Viagra, respectively. The numbers of HSCs in the bloodstream in the rapid 2-hr (2,500 HSCs/mouse) and 3-day (2,800 HSCs/mouse) Viagra/AMD3100 combination were similar to the numbers present 1 day after 4 consecutive days of G-CSF injections (3,400 HSCs/mouse).

### Example 42

### In Vivo Hematopoietic Stem/Progenitor Cell (HSPC) Transduction With RNV

The profile of mouse HSPC was Lin-, Sca1+, Kit1^{HI} (LSK).

The HSPC were harvested from peripheral blood (PB), spleen(S) and bone marrow (BM) after transduction in vivo with high titer (>E8 TU/ml) purified RNV encoding GFP, and characterized by two different assays: colony formation; and FACS analyses.

The mobilization protocol was shown to work in the target animals (Balb/c mice). To mobilize HSPCs, Balb/c mice were used. A first group (Group 1) was mobilized using cyclophosphamide + GCSF + AMD3100 as follows:
Cyclophosphamide + G-CSF + AMD3100 mobilization regimen
Day 0: Cyclophosphamide (CY) (ip) 4 mg/100 µL per 20 g mouse (200 mg/kg/day)
Day 1: G-CSF (sc) 5 µg / µL per 20 g mouse per day (250 µg/kg/day)
Day 2: G-CSF (sc) 5 µg/ µL per 20 g mouse per day (250 µg/kg/day)
Day 3: G-CSF (sc) 5 µg/ µL per 20 g mouse per day (250 µg/kg/day)
Day 4: AMD3100 (sc) 100 µg/ µL per 20 g mouse (5 mg/kg) inject within 12-14 hrs after the last dose of G-CSF
One hour after AMD3100 administration peripheral blood (PB), spleen (S) and bone marrow (BM) was harvested and analyzed by FACS for the presence of HSPC (mouse lin⁻, c-kit⁺, Sca-1⁺) in PB, S and BM.

A second group (Group 2) was not mobilized.

Results of the FACS analyses are summarized in Table 10.

TABLE 10

Summary of results from FACS analyses of mobilization of HSPC (Lin-, Sca1+, Kit1+) in PB, spleen & bone marrow. Individual numbers represent values from individual mice

| Group | mouse status | %LIN⁻ cells that are Sca⁺ Kit⁺ (PB) | %LIN⁻ cells that are Sca⁺ Kit⁺(S) | %LIN⁻ cells that are Sca⁺ Kit⁺(BM) |
|---|---|---|---|---|
| A1 gp.1 | Mobilized | 7.09, 1.9, 3.84 | 3.55, 2.04, 3.34 | 5.17, 4.97, 5.92 |
| A1 gp.2 | Not mobilized | 0.08, 0.57, 0.83 | 2.3, 1.93, 2.07 | 1.00, 0.87, 1.78 |

These experiments showed mobilization of HSPC Into the periphery.

### In Vivo Hematopoietic Stem/Progenitor Cell (HSPC) Transduction

Next, (A2 experiments) experiments were performed as above but followed by retroviral (RV) transduction in vivo for 2 hours. Three groups of Balb/c mice were used: Group 1-mobilized, no RV (n=3 mice #1,2,3); Group 2 – mobilized + RV (n=6 mice #4, 5, 6, 7, 8, 9); and Group 3-not mobilized, no RV (control)(n=1 mouse #10).

Peripheral blood (PB), Spleen (S) and Bone Marrow (BM) were harvested 2 hours after RV transduction. PB, S and BM cells were cultured for 3 days and analyzed by FACS for GFP+ HSPC (LIN⁻ cells that are Sca⁺ Kit⁺). Multilineage reconstitution of GFP⁺ HSPCs was performed by culturing PB, S and BM cells in methylcellulose for 5 days to see if HSPC colonies are GFP⁺.

Next, (A3 experiments) experiments were performed as above but followed by retroviral (RV) transduction in vivo for 2 days. Three groups of Balb/c mice were used: Group 1-mobilized, no RV (n=3 mice #1,2,3); Group 2 – mobilized + RV (n=6 mice #4, 5, 6, 7, 8, 9); and Group 3-not mobilized, no RV (control)(n=1 mouse #10).

Peripheral blood (PB), Spleen (S) and Bone Marrow (BM) were harvested 2 days after RV transduction. PB, S and BM cells were cultured for 3 days and analyzed by FACS for GFP+ HSPC (LIN⁻ cells that are Sca⁺ Kit⁺). Multilineage reconstitution of GFP⁺ HSPCs was performed by culturing PB, S and BM cells in methylcellulose for 10 days to see if HSPC colonies are GFP⁺.

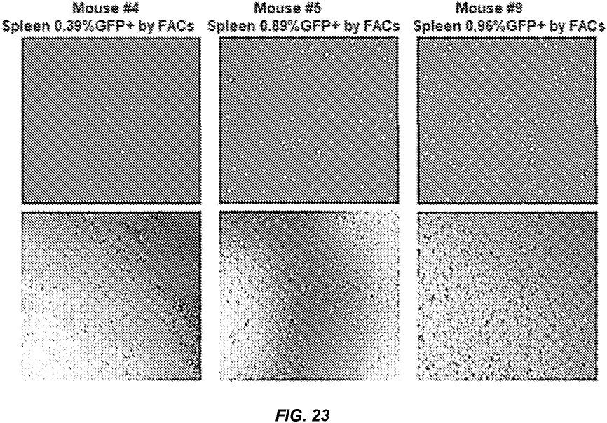

See FIG. 23 for pictures of GFP⁺ cells in spleen. Table 11 shows the result of experiment A2.

TABLE 11

Mobilization and transduction with RNV-GFP of HSPC from PB S, & BM; numbers below 0.1% were counted as zero as that is the limit of reliable detection of the FACS machine

| A2 Group | % cells GFP+ (PB) | % cells that are LIN⁻ Sca⁺Kit⁺GFP⁺ (PB) | % cells that are LIN⁻ Sca⁺Kit⁺GFP⁺ (S) | % cells that are LIN⁻ Sca⁺Kit⁺GFP⁺ (BM) |
|---|---|---|---|---|
| A2 grp1 | 0 | - | - | - |
| A2 grp2 | 0.97;0.75; 0.1;1.03; 1.06 | 0;0;0.23; 0.65 | 0.46;0;0.6 | 0;1.0;0 |
| A2 grp3 | 0 | - | - | - |

Peripheral Blood(PB); CFU: colony forming unit, M: monocyte/macrophage, G: granulocyte, GM: granulocyte/macrophage, GEMM: granulocyte/erythroid/macrophage/ megakaryocyte. 1+2+3: pooled peripheral blood from mobilized but untransduced negative controls, 4 thru 9: peripheral blood from individual mobilized and RNV-transduced mice, 10: peripheral blood from non-mobilized untransduced control:

TABLE 12

(Experiment A2) HPSC mobilization followed by RNV transduction in vivo for 2 hours

| Mouse # | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Pooled 1+2+3 | | | | | 0 | 1494 |
| 4 | 83 | 19 | 35 | 9 | 146 | 4483 |
| 5 | 34 | 16 | 12 | 7 | 69 | 3626 |
| 6 | 8 | 1 | 4 | 1 | 14 | 4572 |
| 7 | 77 | 28 | 13 | 4 | 122 | 6672 |
| 8 | 12 | 26 | 14 | 3 | 55 | 1741 |
| 9 | 27 | 23 | 23 | 1 | 74 | 1345 |
| 10 | | | | | | 15 |

Table 12 shows the numbers of GFP⁺ hematopoietic stem/progenitor cell (HSPC) derived colonies obtained after culturing 4×10⁴ peripheral blood (PB) cells per well in 12-well plate for 5 days in MethoCult GFM3434 (Stem Cell Technologies).

Spleen (S); CFU: colony forming unit, M: monocyte/macrophage, G: granulocyte, GM: granulocyte/ macrophage, GEMM: granulocyte/erythroid/macrophage/ megakaryocyte. 1+2+3: pooled splenocytes from mobilized but untransduced negative controls, 4 thru 9: splenocytes from individual mobilized and RNV-transduced mice (note: no colonies formed from #7, #8 due to technical difficulties), 10: splenocytes from non-mobilized untransduced control:

TABLE 13

(Experiment A2) HPSC mobilization followed by RV transduction in vivo for 2 hours

| Mouse # | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Pooled 1+2+3 | | | | | 0 | -6400 |
| 4 | 19 | 19 | 0 | 2 | 50 | 6000 |
| 5 | 137 | 30 | 19 | 2 | 188 | 8464 |
| 6 | 5 | 6 | 0 | 0 | 11 | 4952 |
| 7 | - | - | - | - | - | - |
| 8 | - | - | - | - | - | - |
| 9 | 98 | 31 | 30 | 1 | 160 | 5840 |
| 10 | - | - | - | - | 0 | 0 |

Table 13 shows the Numbers of GFP+ hematopoietic stem/progenitor cell (HSPC) derived colonies obtained after culturing 2×10⁵ spleen (S) cells per well in 12-well plate for 5 days in MethoCult GFM3434 (Stem Cell Technologies).

Bone Marrow (BM); CFU: colony forming unit, M: monocyte/macrophage, G: granulocyte, GM: granulocyte/macrophage, GEMM: granulocyte/erythroid/macrophage/ megakaryocyte. 1+2+3: pooled bone marrow from mobilized but untransduced negative controls, 4 thru 9: bone marrow from individual mobilized and RV-transduced mice, 10: bone marrow from non-mobilized untransduced control:

TABLE 14

(Experiment A2) HPSC mobilization followed by RV transduction in vivo for 2 hours

| Mouse # | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Pooled 1+2+3 | - | - | - | - | 0 | 904 |
| 4 | - | - | - | - | 0 | 1016 |
| 5 | - | - | - | - | 0 | 1668 |
| 6 | - | - | - | - | 9 | 912 |
| 7 | - | 1 | - | - | 1 | 544 |
| 8 | 3 | - | - | - | 3 | 1600 |
| 9 | 1 | - | - | - | 1 | 1808 |
| 10 | - | - | - | - | 0 | 472 |

Table 14 shows the numbers of GFP⁺ hematopoietic stem/progenitor cell (HSPC)-derived colonies obtained after culturing 3×10⁴ bone marrow (BM) cells per well in 12-well plate for 5 days in MethoCult GF M3434 (StemCell Technologies).

CFU: colony forming unit, M: monocyte/macrophage, G: granulocyte, GM: granulocyte/macrophage, GEMM: granulocyte/erythroid/macrophage/megakaryocyte. 1+2+3: pooled peripheral blood from mobilized but untransduced negative controls, 4 thru 9: peripheral blood from individual mobilized and RV-transduced mice (note: #5 died after RNV injection and was removed from analysis), 10: peripheral blood from non-mobilized untransduced control:

TABLE 15

(Experiment A3) HPSC mobilization followed by RNV transduction in vivo for 2 days

| Mouse # | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Pooled 1+2+3 | - | - | - | - | 0 | 76 |
| 4 | 1 | 0 | 0 | 0 | 1 | 130 |
| 5-died | - | - | - | - | - | - |
| 6 | 0 | 1 | 0 | 0 | 1 | 131 |
| 7 | 2 | 0 | 0 | 0 | 2 | 83 |
| 8 | 2 | 3 | 2 | 0 | 7 | 127 |
| 9 | 0 | 0 | 0 | 0 | 0 | 147 |
| 10 | - | - | - | - | 0 | 1 |

Table 15 shows the numbers of GFP⁺ hematopoietic stem/progenitor cell (HSPC)-derived colonies obtained after culturing 4×10⁴ peripheral blood (PB) cells per well in 12-well plate for 7 days in MethoCult GF M3434 (StemCell Technologies).

Spleen (S); CFU: colony forming unit, M: monocyte/macrophage, G: granulocyte, GM: granulocyte/ macrophage, GEMM: granulocyte/erythroid/macrophage/ megakaryocyte. 1+2+3: pooled splenocytes from mobilized but untransduced negative controls, 4 thru 9: splenocytes from individual mobilized and RV-transduced mice (note: #5 died after RV injection and was removed from analysis; no colonies formed from #8), 10: splenocytes from non- mobilized untransduced control (also no colonies observed):

TABLE 16

(Experiment A3) HPSC mobilization followed by RV transduction in vivo for 2 days

| Mouse # | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Pooled 1+2+3 | - | - | - | - | 0 | 436 |
| 4 | 4 | 1 | 1 | 0 | 6 | 514 |
| 5-died | - | - | - | - | - | - |
| 6 | 5 | 1 | 1 | 0 | 7 | 392 |
| 7 | 12 | 5 | 2 | - | 19 | 772 |
| 8 | - | - | - | - | - | - |
| 9 | 0 | 0 | 0 | 0 | 0 | 459 |
| 10 | - | - | - | - | - | 90 |

Table 16 shows the numbers of GFP⁺ hematopoietic stem/progenitor cell (HSPC)-derived colonies obtained after culturing 2×10⁵ spleen (S) cells per well in 12-well plate for 7 days in MethoCult GF M3434 (StemCell Technologies.

Bone Marrow (BM); CFU: colony forming unit, M: monocyte/macrophage, G: granulocyte, GM: granulocyte/macrophage, GEMM: granulocyte/erythroid/macrophage/ megakaryocyte. 1+2+3: pooled bone marrow from mobilized but untransduced negative controls, 4 thru 9: bone marrow from individual mobilized and RV-transduced mice (note: #5 died after RNV injection and was removed from analysis), 10: bone marrow from non-mobilized untransduced control.

TABLE 17

(Experiment A3) HPSC mobilization followed by RNV transduction in vivo for 2 days

| Mouse # | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Pooled 1+2+3 | - | - | - | - | 0 | 24 |
| 4 | 1 | 0 | 0 | 0 | 1 | 41 |
| 5-died | - | - | - | - | - | - |
| 6 | 0 | 0 | 0 | 0 | 0 | 82 |
| 7 | 0 | 0 | 0 | 0 | 0 | 138 |
| 8 | 0 | 0 | 0 | 0 | 0 | 0 |
| 9 | 0 | 0 | 0 | 0 | 0 | 69 |
| 10 | - | - | - | - | 0 | 152 |

Table 17 shows the numbers of GFP⁺ hematopoietic stem/progenitor cell (HSPC)-derived colonies obtained after culturing 3×10⁴ bone marrow (BM) cells per well in 12-well plate for 7 days in MethoCult GF M3434 (StemCell Technologies).

These experiments (A2 and A3) show that in vivo (IV) delivery of about 2E7 TU’s of RNV in 100-200 µL can yield up to ~7% of HSPC derived CFUs being transduced.

An additional experiment (Experiment B1) was performed to analyze CD34+ cell transduction. Two groups of CD34+ cells were used: Group 1 – CD34+, no RV (1 well in 234 well-plate); Group 2 –CD34+ plus RV (2 wells in 24 well-place). Cells were thawed, cultured and expanded in serum-free SFEMII+CC100 cytokine cocktail (StemCell). Cells were plated (3 wells in 24-well plate) with 100 µl of CD34+ cells (3×10⁵), and 100 µl of RNV-GFP virus was added to the group 2 wells in a total volume of 2 ml.

Cells were collected in 15 ml tubes and washed with PBS, replated into new wells in 24 well plate. The cells were cultured for 2 additional days in BBMM _ CC100 cytokine expansion factors and analyzed by FACS for the presence of GFP-positive CD34+ cells. In addition. Mutlilineage reconstitution of GFP+ HSPCs by culturing CD34+ cells in methylcellulose for 15 days was performed to see if HSPC colonies are GPF+.

transduction control study; CFU: colony forming unit, M: monocyte/macrophage, G: granulocyte, GM: granulocyte/macrophage, GEMM: granulocyte/erythroid/macrophage/ megakaryocyte. (untransduced: untransduced control CD34+ cells; RV TD 1, 2: RV transduction replicates):

TABLE 18

(Experiment B1) Human CD34+ HSPC in vitro RNV transduction for 2 hours

| Human CD34+ | GFP⁺ CFU-M | GFP⁺ CFU-G | GFP⁺ CFU-GM | GFP⁺ CFU-GEMM | Total GFP⁺ Colonies | Total Colonies |
|---|---|---|---|---|---|---|
| Untransduced | - | - | - | - | - | 248 |
| RV TD1 | 6 | 0 | 1 | 0 | 7 | 254 |
| RV TD2 | 10 | 2 | 2 | 0 | 7 | 252 |

Table 18 shows the numbers of GFP+ hematopoietic stem/progenitor cell (HSPC)-derived colonies obtained after culturing 5×102 human CD34+ cells per 35 mm dish for 15 days in MethoCult Classic (GF H4434, StemCell Technologies).

This experiment (B1) shows that the preparations of RNV that give in vivo transduction of mouse stem cells, are also capable of transducing human CD34+ HSPC.

A number of embodiments have been set forth above to illustrate the disclosure. The following claims further set forth what the Applicants regard as their invention.

What is claimed is:

1. A recombinant vector comprising:
(a) a polynucleotide encoding a chimeric antigen receptor (CAR); and
(b) a polynucleotide comprising at least one miRNA targeting sequence, wherein (a) and (b) are linked on the same polynucleotide.

2. The recombinant vector of claim 1, further comprising (c) a polynucleotide encoding a cytotoxic polypeptide that converts a prodrug to a cytotoxic drug.

3. The recombinant vector of claim 1, wherein (a) comprises
a first polynucleotide domain encoding one or more antigen binding domain(s);
an optional polynucleotide domain encoding a linker; and
a second polynucleotide domain operably linked to the first polynucleotide domain, wherein the second polynucleotide domain encodes a transmembrane domain; and
a third polynucleotide domain encoding an intracellular signaling domain.

4. The recombinant vector of claim 3, wherein the first polynucleotide domain encodes an antibody fragment, single domain antibody, single chain variable fragment, single domain antibody, camelid VHH domain, a non-immunoglobulin antigen binding scaffold, a receptor or receptor fragment, or a bispecific antibody.

5. (canceled)

6. The recombinant vector of claim 3, wherein the transmembrane domain is from a member selected from the group consisting alpha, beta or zeta chain of a T-cell receptor, CD3y, CD3ε, CD3δ, CD28, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI la, CD18), ICOS (CD278), 4-1BB (CD137), GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CDI Id, ITGAE, CD103, ITGAL, CDI la, LFA-1, ITGAM, CDI Ib, ITGAX, CDI Ic, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

7. The recombinant vector of claim 3, wherein the third polynucleotide domain encodes an intracellular signaling domain selected from the group consisting of CD3 zeta, common FeR gamma (FCER1G), Fe gamma Rlla, FeR beta (Fe Epsilon R1b), CD3 gamma, CD3 delta, CD3 epsilon, CD79a, CD79b, DAPIO, and DAP12.

8. The recombinant vector of claim 2, wherein the cytotoxic polypeptide that converts a prodrug to a cytotoxic drug is selected from the group consisting of a polypeptide having cytosine deaminase activity, a polypeptide having thymidine kinase activity and a combination thereof.

9. The recombinant vector of claim 1, wherein the vector is an integrating vector.

10. The recombinant vector of claim 9, wherein the vector is a retroviral vector.

11. The recombinant vector of claim 10, wherein the retroviral vector is a non-replicating gammaretroviral vector.

12. The recombinant vector of claim 1, wherein the at least one miRNA targeting sequence is bound by an miRNA selected from the group consisting of hsa-miR-223-3p, hsa-miR143-3p, hsa-mir182-5p, hsa-miR-10bp, hsa-miR141-3p, has-miR486-5p and any combination of the foregoing.

13. A recombinant retroviral particle comprising:
a gag polypeptide;
a pol polypeptide;
an env polypeptide; and
a retroviral polynucleotide contained within a capsid of the retroviral vector, wherein the retroviral polynucleotide comprises from 5′ to 3′: (R-U5 domain)-(optional signal peptide coding sequence domain)-(Binding domain coding sequence domain)-(optional hinge/linker coding sequence domain)-(transmembrane (TM) coding sequence domain)-(miRNA target domain(s))-(U3-R domain).

14. The recombinant retroviral particle of claim 13, wherein the R-U5 domain has a sequence that is at least 80% identical to SEQ ID NO:25 from nucleotide 1 to about nucleotide 145.

15. The recombinant retroviral particle of claim 13, wherein the binding domain coding sequence is preceded by a signal sequence.

16. (canceled)

17. The recombinant retroviral particle of claim 13, wherein the retroviral polynucleotide further comprises a kill switch domain coding sequence.

18. The recombinant retroviral particle of claim 17, wherein the kill switch coding domain comprises an IRES operably linked to a coding sequence for a polypeptide that converts a prodrug to a cytotoxic drug.

19. The recombinant retroviral particle of claim 18, wherein the polypeptide has thymidine kinase (TKO) activity or cytosine deaminase (CD) activity.

20. The recombinant retroviral particle of claim 13, wherein the retroviral polynucleotide comprises at least one miRNA targeting sequence.

21. The recombinant retroviral particle of claim 20, wherein the at least one miRNA targeting sequence comprises a plurality of miRNA targeting sequences.

22. The recombinant retroviral particle of claim 21, wherein the plurality of miRNA targeting sequences are the same.

23. Ther recombinant retroviral particle of claim 21, wherein at least two of the plurality of miRNA targeting sequences are different.

24. The recombinant retroviral particle of claim 13, wherein the U3-R domain comprises a sequence that is at least 80% identical to SEQ ID NO:25 from about nucleotide 5537 to about 6051.

25. The recombinant retroviral particle of claim 13, wherein each domain can be separated from another domain by small spacer sequences of about 1-20 nucleotides.

26. A pharmaceutical composition comprising the recombinant retroviral particle of claim 13 in a pharmaceutically acceptable carrier.

27. A composition comprising:
(i) a retroviral vector comprising a polynucleotide encoding an antigen binding receptor construct targeting an antigen; and
(ii) an miR-targeting sequence or miR-targeting cassette;
(iii) an optional kills switch coding domain; and
(iv) a pharmaceutically acceptable carrier.

28. The composition of claim 27, wherein the antigen binding receptor construct comprises a binding domain, an optional hinge/linker domain, a transmembrane domain, and an intracellular signaling domain.

29. The composition of claim 28, wherein the binding domain comprises an antibody, a Fv, a Fab, a (Fab′)2, a heavy chain variable region of an antibody (vH domain), a light chain variable region of an antibody (vL domain), a single domain antibody, a single chain variable fragment (scFv), a monomeric variable region of an antibody, a camelid vHH domain, a non-immunoglobulin antigen binding domain (e.g., DARPIN, an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein), a ligand or a fragment thereof having specificity to an antigen associated with a disease or disorder.

30. The composition of claim 27, wherein the retroviral vector comprises a lipid envelope.

31. The composition of claim 30, wherein the envelope is amphotrophic.

32. The composition of claim 27, wherein the antigen binding receptor construct comprises a chimeric antigen receptor.

33. The composition of claim 27, wherein the miR-targeting sequence or miR-targeting cassette comprises one or more targeting sequence for miRNA that are expressed in off-target cells.

34. The composition of claim 27, wherein the optional kills switch comprises a coding sequence from a suicide gene.

35. The composition of claim 34, wherein the suicide gene encodes a polypeptide having cytosine deaminase activity or thymidine kinase activity.

36. The composition of claim 27, wherein the polynucleotide comprises from 5′ to 3′: R-U5-(antigen binding receptor coding sequence)-(miR targeting sequence or cassette)-(optional suicide gene)-U3-R.

37. (canceled)

38. The composition of claim 29, where the antigen is selected from the group consisting of: CD5; CD19; CD123; CD20; CD22; CD24; CD30; CD33, CD34; CD38, CD72; CD97; CD171; CS1 (also referred to as CD2 subset 1, CRACC, MPL, SLAMF7, CD319, and 19A24); C-type lectin-like molecule-1 (CLL-1 or CLECL1); epidermal growth factor receptor variant III (EGFRviii); ganglioside G2 (GD2); ganglioside GD3 (aNeu5Ac(2-8)aNeu5Ac(2-3)bDGalp(I-4 )bDGIcp(I-I)Cer); TNF receptor family member B cell maturation (BCMA); Tn antigen ((Tn Ag) or (GaINAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD44v6; a glycosylated CD43 epitope; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; Platelet-derived growth factor receptor beta (PDGFR-beta); Stage-specific embryonic antigen-4 (SSEA-4); Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gpl00); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(l-4)bDGlcp(l-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51 E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1 ); ETS translocation-variant gene 6, located on chromosome 12p (ETV6-AML); sperm protein 17 (SPA17); X Antigen Family, Member IA (XAGEI); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin Bl; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAX5); proacrosin binding protein sp32 (OY-TESI); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLLI); MPL; Biotin; c-MYC epitope Tag; LAMP1 TROP2; GFRalpha4; CDH17; CDH6; NYBR1; CDH19; CD200R; Slea (CA19.9; Sialyl Lewis Antigen); Fucosyl-GM1; PTK7; gpNMB; CDH1-CD324; DLL3; CD276/B7H3; IL11Ra; IL13Ra2; CD179b-IGLI1; TCRgamma-delta; NKG2D; CD32 (FCGR2A); Tn ag; Tim1-/HVCR1; CSF2RA (GM-CSFR-alpha); TGFbetaR2; Lews Ag; TCR-beta1 chain; TCR-beta2 chain; TCRgamma chain; TCR-delta chain; FITC; Leutenizing hormone receptor (LHR); Follicle stimulating hormone receptor (FSHR) ; Gonadotropin Hormone receptor (CGHR or GR); CCR4; GD3; SLAMF6; SLAMF4; HIV1 envelope glycoprotein; HTLV1-Tax; CMV pp65; EBV-EBNA3c; KSHV K8.1; KSHV-gH; influenza A hemagglutinin (HA); GAD; PDL1; Guanylyl cyclase C (GCC); auto antibody to desmoglein 3 (Dsg3); auto antibody to desmoglein 1 (Dsg1); HLA; HLA-A; HLA-A2; HLA-B; HLA-C; HLA-DP; HLA-DM; HLA-DOA; HLA-DOB; HLA-DQ; HLA-DR; HLA-G; IgE; CD99; Ras G12V; Tissue Factor 1 (TF1); AFP; GPRC5D; Claudin18.2 (CLD18A2 or CLDN18A.2); P-glycoprotein; STEAP1; Liv1; Nectin-4; Cripto; gpA33; BST1/CD157; low conductance chloride channel; and the antigen recognized by TNT antibody.

39. (canceled)

40. A method of providing anti-disease immunity in a subject comprising administering to the subject an effective amount of the composition of claim 26, wherein the composition transduces cells in vivo to give rise to immune effector cells that express an antigen receptor binding construct.

41. The method of claim 40, wherein the composition comprises from 10³ to 10¹¹ vector transforming units (TU) per dose.

42. The method of claim 40, wherein the antigen receptor binding construct is not expressed in cells that express an miRNA that binds to the miRNA target sequence of the polynucleotide.

43. The method of claim 40, wherein the anti-disease immunity treats a disease selected from the group consisting of a proliferative disease, a precancerous condition, a cancer, and a non-cancer related indication associated with expression of a disease-associated antigen to which the antigen receptor binding construct binds.

44-45. (canceled)

46. The method of claim 40, wherein the vector transduces cells selected from T-lymphocytes (T-Cells), naïve T-cells, memory T-Cells, Tregs, NK cells, hematopoietic stem cells, and any combination thereof.

47. The composition of claim 26, wherein the retroviral vector comprises the sequence of SEQ ID NO:25.

48. A method of producing a retroviral vector comprising transforming a cell line expressing murine leukemia virus (MLV) gag-pol genes with a vector of claim 1.

49. The method of claim 48, wherein the vector comprises a sequence as set forth in SEQ ID NO: 1-23 or 24.

50-51. (canceled)

52. A method of mobilizing stem cells for in vivo gene transfer therapy, comprising
administering to a subject an agent that mobilizes hematopoietic stem cells from the bone marrow.

53. The method of claim 52, wherein the agent is administered prior to administering a vector comprising a transgene for in vivo transduction of hematopoietic stem cells.

54. The method of claim 52, wherein the agent is selected from the group consisting of plerixafor, G-CSF, Sildenafil, filgrastim, lenograstim, GM-CSF, molgramostim, sargramostim and any combination thereof.

55. (canceled)


SEQUENCE LISTING (28 sequences)

SEQ ID NO: 1 - RNA, 1662 nt, Artificial Sequence
  pBA-9b vRNA genome with multiple cloning site
gcgccaguccuccgauugacugagucgcccggguacccguguauccaauaaacccucuugcaguugcauccgacuuguggucucgcuguuccuugggagggucuccucugagugauugacuacccgucagcgggggucuuucauuugggggcucguccgggaucgggagaccccugcccagggaccaccgacccaccaccgggagguaagcuggccagcaacuuaucugugucuguccgauugucuagugucuaugacugauuuuaugcgccugcgucgguacuaguuagcuaacuagcucuguaucuggcggacccgugguggaacugacgaguucggaacacccggccgcaacccugggagacgucccagggacuucgggggccguuuuuguggcccgaccugaguccaaaaaucccgaucguuuuggacucuuuggugcaccccccuuagaggagggauaugugguucugguaggagacgagaaccuaaaacaguucccgccuccgucugaauuuuugcuuucgguuugggaccgaagccgcgccgcgcgucuugucugcugcagcaucguucuguguugucucugucugacuguguuucuguauuugucugagaauuaaggccagacuguuaccacucccugaaguuugaccuuaggucacuggaaagaugucgagcggaucgcucacaaccagucgguagaugucaagaagagacguuggguuaccuucugcucugcagaauggccaaccuuuaacgucggauggccgcgagacggcaccuuuaaccgagaccucaucacccagguuaagaucaaggucuuuucaccuggcccgcauggacacccagaccagguccccuacaucgugaccugggaagccuuggcuuuugaccccccucccugggucaagcccuuuguacacccuaagccuccgccuccucuuccuccauccgccccgucucucccccuugaaccuccucguucgaccccgccucgauccucccuuuauccagcccucacuccuucucuaggcgccggaauuaauucucgaggggcccagaucugcggccgcucgcgagucgacaagcuuggauccaucgauaaaauaaaagauuuuauuuagucuccagaaaaaggggggaaugaaagaccccaccuguagguuuggcaagcuagcuuaaguaacgccauuuugcaaggcauggaaaaauacauaacugagaauagagaaguucagaucaaggucaggaacagauggaacagcugaauaugggccaaacaggauaucugugguaagcaguuccugccccggcucagggccaagaacagauggaacagcugaauaugggccaaacaggauaucugugguaagcaguuccugccccggcucagggccaagaacagaugguccccagaugcgguccagcccucagcaguuucuagagaaccaucagauguuuccagggugccccaaggaccugaaaugacccugugccuuauuugaacuaaccaaucaguucgcuucucgcuucuguucgcgcgcuucugcuccccgagcucaauaaaagagcccacaaccccucacucggcgcgccaguccuccgauugacugagucgcccggguacccguguauccaauaaacccucuugcaguugca

SEQ ID NO: 2 - DNA, 5640 bp, Artificial Sequence
  Construct 7: pBA-9b-GFPmiR 223-3p4TX
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgagatggccagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatggggtatttgacaaactgacacgtatggggtatttgacaaactgacatcgatggggtatttgacaaactgacatcactggggtatttgacaaactgacagcggccgctcgcgagtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 8395 bp, Artificial Sequence
  Construct 16: pBA-9b-hCD19CAR miRTCMVpromIL12
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgactggggtatttgacaaactgacacgtatggggtatttgacaaactgacatcgatggggtatttgacaaactgacatcactggggtatttgacaaactgacatgctgggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaaggccaccatgtggccccccggcagcgccagccagcccccccccagccccgccgccgccaccggcctgcaccccgccgccagacccgtgagcctgcagtgcagactgagcatgtgccccgccagaagcctgctgctggtggccaccctggtgctgctggaccacctgagcctggccagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagcggcagcaccagcggcagcggcctgcccggcagcggcggcggcagcaccctgggcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagcgatcaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 8380 bp, Artificial Sequence
  Construct 15: pBA-9b-hCD19CAR miRTCMVpromIL7R-CPT
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgactggggtatttgacaaactgacacgtatggggtatttgacaaactgacatcgatggggtatttgacaaactgacatcactggggtatttgacaaactgacagtcctgtgcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaaggccaccatgaccatcctgggcaccaccttcggcatggtgttcagcctgctgcaggtggtgagcggcgagagcggctacgcccagaacggcgacctggaggacgccgagctggacgactacagcttcagctgctacagccagctggaggtgaacggcagccagcacagcctgacctgcgccttcgaggaccccgacgtgaacatcaccaacctggagttcgagatctgcggcgccctggtggaggtgaagtgcctgaacttcagaaagctgcaggagatctacttcatcgagaccaagaagttcctgctgatcggcaagagcaacatctgcgtgaaggtgggcgagaagagcctgacctgcaagaagatcgacctgaccaccatcgtgaagcccgaggcccccttcgacctgagcgtggtgtacagagagggcgccaacgacttcgtggtgaccttcaacaccagccacctgcagaagaagtacgtgaaggtgctgatgcacgacgtggcctacagacaggagaaggacgagaacaagtggacccacgtgaacctgagcagcaccaagctgaccctgctgcagagaaagctgcagcccgccgccatgtacgagatcaaggtgagaagcatccccgaccactacttcaagggcttctggagcgagtggagccccagctactacttcagaacccccgagatcaacaacagcagcggcgagatggaccccatcctgctgacctgccccaccatcagcatcctgagcttcttcagcgtggccctgctggtgatcctggcctgcgtgctgtggaagaagagaatcaagcccatcgtgtggcccagcctgcccgaccacaagaagaccctggagcacctgtgcaagaagcccagaaagaacctgaacgtgagcttcaaccccgagagcttcctggactgccagatccacagagtggacgacatccaggccagagacgaggtggagggcttcctgcaggacaccttcccccagcagctggaggagagcgagaagcagagactgggcggcgacgtgcagagccccaactgccccagcgaggacgtggtgatcacccccgagagcttcggcagagacagcagcctgacctgcctggccggcaacgtgagcgcctgcgacgcccccatcctgagcagcagcagaagcctggactgcagagagagcggcaagaacggcccccacgtgtaccaggacctgctgctgagcctgggcaccaccaacagcaccctgcccccccccttcagcctgcagagcggcatcctgaccctgaaccccgtggcccagggccagcccatcctgaccagcctgggcagcaaccaggaggaggcctacgtgaccatgagcagcttctaccagaaccaggatcaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 7456 bp, Artificial Sequence
  Construct 14: pBA-9b-hCD19CAR miRTCMVpromIL2wt
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgactggggtatttgacaaactgacacgtatggggtatttgacaaactgacatcgatggggtatttgacaaactgacatcactggggtatttgacaaactgacagtcgaccgtctggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaaggccaccatgtacagaatgcagctgctgagctgcatcgccctgagcctggccctggtgaccaactctgcccccaccagcagcagcaccaagaagacccagctgcagctggagcacctgctgctggacctgcagatgatcctgaacggcatcaacaactacaagaaccccaagctgaccagaatgctgaccttcaagttctacatgcccaagaaggccaccgagctgaagcacctgcagtgcctggaggaggagctgaagcccctggaggaggtgctgaacctggcccagagcaagaacttccacctgagacccagagacctgatcagcaacatcaacgtgatcgtgctggagctgaagggcagcgagaccaccttcatgtgcgagtacgccgacgagaccgccaccatcgtggagttcctgaacagatggatcaccttctgccagagcatcatcagcaccctgaccgatcaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 7560 bp, Artificial Sequence
  Construct 13: pBA-9b-hCD19CAR miRTCMVpromIL2F42A
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacgcggtcccgcggcgccccgcctcgatgcggtcccgcggcgccccgcctgcatgcggtcccgcggcgccccgcctctacgcggtcccgcggcgccccgcctattatggggtatttgacaaactgacacgtatggggtatttgacaaactgacatcgatggggtatttgacaaactgacatcactggggtatttgacaaactgacagtcgaccgtctggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaaggccaccatgtacagaatgcagctgctgagctgcatcgccctgagcctggccctggtgaccaactctgcccccaccagcagcagcaccaagaagacccagctgcagctggagcacctgctgctggacctgcagatgatcctgaacggcatcaacaactacaagaaccccaagctgaccagaatgctgaccgccaagttctacatgcccaagaaggccaccgagctgaagcacctgcagtgcctggaggaggagctgaagcccctggaggaggtgctgaacctggcccagagcaagaacttccacttcgaccccagagacgtggtgagcaacatcaacgtgttcgtgctggagctgaagggcagcgagaccaccttcatgtgcgagtacgccgacgagaccgccaccatcgtggagttcctgaacagatggatcaccttctgccagagcatcatcagcaccctgaccgatcaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 7984 bp, Artificial Sequence
  Construct 12: pBA-9b-hCD19CAR miRTCMVprom-cJun
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgactggggtatttgacaaactgacacgtatggggtatttgacaaactgacatcgatggggtatttgacaaactgacatcactggggtatttgacaaactgacatgcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggccaccatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgagatcaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 7672 bp, Artificial Sequence
  Construct 10: pBA-9b-hCD19CAR-IRES-TKmiRT+CMVprom-IL15
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgactgcgcggtcccgcggcgccccgcctcgatgcggtcccgcggcgccccgcctgcatgcggtcccgcggcgccccgcctctacgcggtcccgcggcgccccgcctattatggggtatttgacaaactgacacgtatggggtatttgacaaactgacatcgatggggtatttgacaaactgacatcactggggtatttgacaaactgacagtcgacaacttgtttattgcagcttataatggttacaaataaagcaatagcatcacaaatttcacaaataaagcatttttttcactgcattctagttgtggtttgtccaaactcatcaatgtatcttatcatgtctggatctgcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgccaccatggcccccagaagagccagaggctgcagaaccctgggcctgcccgccctgctgctgctgctgctgctgagaccccccgccaccagaggcaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggaggacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctaagactagaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 8424 bp, Artificial Sequence
  Construct 19: pBA-9b-hCD19CAR-IRES-TKmiRT+U6-PD1shRNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggcttcatatccttgccaccaacatgcttccgctttcgaccaagccgcacggtctaggggccacaataatcgccggactgccctgcggcctcggagacagcagaaggcaaccgaagtcaggctcgagcaaaagatgccaaccctcctgcgggtctatatcgatggaccccatggaatggggaagaccactaccacacaactcctggtggcactcggtagccgggacgacatcgtctacgtgcccgaacccatgacttactggcgggttctcggtgcttccgagacaatcgccaatatctacacaacccaacaccgcctcgatcaaggagaaattagcgcaggggacgctgccgtggtgatgacatcagcccaaatcaccatgggaatgccctacgccgtcaccgatgctgtcctggcaccacacattggcggagaggccgggtcaagtcatgcaccaccaccagccctgactatctttctcgaccggcatccaattgcattcatgctgtgctatcctgccgcacgctacctgatgggaagtatgacaccacaggccgtcctcgccttcgttgctctgatccctccaaccctgcctggcactaacatcgttctcggcgcactccccgaagacagacacattgatcggctggccaagaggcaacggcctggcgagagactcgatctggctatgctggctgctattaggagagtgtacgggctgctggccaatactgtgagatacctccaagggggaggaagctggcgcgaggattggggccaactgtctggcgctgctgtgccacctcaaggcgccgagccacagtcaaatgctggtcctaggccccacatcggcgatactctctttacactgttccgggcaccagagctgctcgcacctaatggagatctgtacaatgttttcgcttgggccctcgatgtcctggctaagcggctccggcctatgcacgtgttcatcctcgactacgaccagagcccagctggttgtcgggatgctctcctgcaactgaccagcgggatggtgcagacacacgttactactcccggctccatccccactatctgtgacctcgcccggacatttgcccgggaaatgggcgaagccaactgatcgttggggtatttgacaaactgacacgtatggggtatttgacaaactgacatcgatggggtatttgacaaactgacatcactggggtatttgacaaactgacagtcgacgatcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacagatctgctattatattataattaatacctgacccatattaattataatataatagctttttggtaccgaaacaccgaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 8420 bp, Artificial Sequence
  Construct 48: pBA-9b-hCD19CAR-IRES-TKmiRT+U6-PBCP1shRNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggcttcatatccttgccaccaacatgcttccgctttcgaccaagccgcacggtctaggggccacaataatcgccggactgccctgcggcctcggagacagcagaaggcaaccgaagtcaggctcgagcaaaagatgccaaccctcctgcgggtctatatcgatggaccccatggaatggggaagaccactaccacacaactcctggtggcactcggtagccgggacgacatcgtctacgtgcccgaacccatgacttactggcgggttctcggtgcttccgagacaatcgccaatatctacacaacccaacaccgcctcgatcaaggagaaattagcgcaggggacgctgccgtggtgatgacatcagcccaaatcaccatgggaatgccctacgccgtcaccgatgctgtcctggcaccacacattggcggagaggccgggtcaagtcatgcaccaccaccagccctgactatctttctcgaccggcatccaattgcattcatgctgtgctatcctgccgcacgctacctgatgggaagtatgacaccacaggccgtcctcgccttcgttgctctgatccctccaaccctgcctggcactaacatcgttctcggcgcactccccgaagacagacacattgatcggctggccaagaggcaacggcctggcgagagactcgatctggctatgctggctgctattaggagagtgtacgggctgctggccaatactgtgagatacctccaagggggaggaagctggcgcgaggattggggccaactgtctggcgctgctgtgccacctcaaggcgccgagccacagtcaaatgctggtcctaggccccacatcggcgatactctctttacactgttccgggcaccagagctgctcgcacctaatggagatctgtacaatgttttcgcttgggccctcgatgtcctggctaagcggctccggcctatgcacgtgttcatcctcgactacgaccagagcccagctggttgtcgggatgctctcctgcaactgaccagcgggatggtgcagacacacgttactactcccggctccatccccactatctgtgacctcgcccggacatttgcccgggaaatgggcgaagccaactgatcgttggggtatttgacaaactgacacgtatggggtatttgacaaactgacatcgatggggtatttgacaaactgacatcactggggtatttgacaaactgacagtcgacgatcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacggatccgccacagcagcacagaattctttcaagagaagaattctgtgctgctgtggcttttttagaaacaccgaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 5640 bp, Artificial Sequence
  Construct 51: pBA-9B-emdGFPmiRT199a-4TX
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgagatggccagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagaacaggtagtctgaacactggcgatgaacaggtagtctgaacactgggcatgaacaggtagtctgaacactggctacgaacaggtagtctgaacactgggcggccgctcgcgagtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 5540 bp, Artificial Sequence
  Construct 45: pBA-9b- GFP
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgagatggccagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagcggccgctcgcgagtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 9519 bp, Artificial Sequence
  Construct 41: pBA-9b mCD19CAR-IRES yCD-CMVprom-mIL12P70
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatgggcgtgcccacccagctgctgggcctgctgctgctgtggatcaccgacgccatctgcgacatccagatgacccagagccctgccagcctgagcaccagcctgggcgagaccgtgaccatccagtgccaggccagcgaggacatctacagcggcctggcctggtaccagcagaagcctggcaagagccctcagctgctgatctacggcgccagcgacctgcaggacggcgtgcctagcaggttcagcggcagcggcagcggcacccagtacagcctgaagatcaccagcatgcagaccgaggacgagggcgtgtacttctgccagcagggcctgacctaccctaggaccttcggcggcggcaccaagctggagctgaagggcggcggcggcagcggcggcggcggcagcggcggcggcggcagcgaggtgcagctgcagcagagcggcgccgagctggtgaggcctggcaccagcgtgaagctgagctgcaaggtgagcggcgacaccatcaccttctactacatgcacttcgtgaagcagaggcctggccagggcctggagtggatcggcaggatcgaccctgaggacgagagcaccaagtacagcgagaagttcaagaacaaggccaccctgaccgccgacaccagcagcaacaccgcctacctgaagctgagcagcctgaccagcgaggacaccgccacctacttctgcatctacggcggctactacttcgactactggggccagggcgtgatggtgaccgtgagcagcattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctgagtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaaattagtcgactgcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgccacccaagatgggtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccgtccgatccggtggcggtggctcgggcggtggtgggtcgggtggcggcggatctagggtcattccaagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaatctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 8351 bp, Artificial Sequence
  Construct 42: pBA-9b-mCD19CAR-IRES-TKmiRT+CMVprom-IL2F42A
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatgggcgtgcccacccagctgctgggcctgctgctgctgtggatcaccgacgccatctgcgacatccagatgacccagagccctgccagcctgagcaccagcctgggcgagaccgtgaccatccagtgccaggccagcgaggacatctacagcggcctggcctggtaccagcagaagcctggcaagagccctcagctgctgatctacggcgccagcgacctgcaggacggcgtgcctagcaggttcagcggcagcggcagcggcacccagtacagcctgaagatcaccagcatgcagaccgaggacgagggcgtgtacttctgccagcagggcctgacctaccctaggaccttcggcggcggcaccaagctggagctgaagggcggcggcggcagcggcggcggcggcagcggcggcggcggcagcgaggtgcagctgcagcagagcggcgccgagctggtgaggcctggcaccagcgtgaagctgagctgcaaggtgagcggcgacaccatcaccttctactacatgcacttcgtgaagcagaggcctggccagggcctggagtggatcggcaggatcgaccctgaggacgagagcaccaagtacagcgagaagttcaagaacaaggccaccctgaccgccgacaccagcagcaacaccgcctacctgaagctgagcagcctgaccagcgaggacaccgccacctacttctgcatctacggcggctactacttcgactactggggccagggcgtgatggtgaccgtgagcagcattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctgagtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaaattagtcgactgcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgccaccatggtaagcgctattgttttatatgtgcttttggcggcggcggcgcattctgcctttgcggcggatccagcacctacttcaagttctacaaagaaaacacagctacaactggagcatttacttctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaactcatcatcaccatcaccattgatctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 9252 bp, Artificial Sequence
  Construct 9: pBA-9b-hCD19CAR miRTCMVpromIL15reverse
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggcttcatatccttgccaccaacatgcttccgctttcgaccaagccgcacggtctaggggccacaataatcgccggactgccctgcggcctcggagacagcagaaggcaaccgaagtcaggctcgagcaaaagatgccaaccctcctgcgggtctatatcgatggaccccatggaatggggaagaccactaccacacaactcctggtggcactcggtagccgggacgacatcgtctacgtgcccgaacccatgacttactggcgggttctcggtgcttccgagacaatcgccaatatctacacaacccaacaccgcctcgatcaaggagaaattagcgcaggggacgctgccgtggtgatgacatcagcccaaatcaccatgggaatgccctacgccgtcaccgatgctgtcctggcaccacacattggcggagaggccgggtcaagtcatgcaccaccaccagccctgactatctttctcgaccggcatccaattgcattcatgctgtgctatcctgccgcacgctacctgatgggaagtatgacaccacaggccgtcctcgccttcgttgctctgatccctccaaccctgcctggcactaacatcgttctcggcgcactccccgaagacagacacattgatcggctggccaagaggcaacggcctggcgagagactcgatctggctatgctggctgctattaggagagtgtacgggctgctggccaatactgtgagatacctccaagggggaggaagctggcgcgaggattggggccaactgtctggcgctgctgtgccacctcaaggcgccgagccacagtcaaatgctggtcctaggccccacatcggcgatactctctttacactgttccgggcaccagagctgctcgcacctaatggagatctgtacaatgttttcgcttgggccctcgatgtcctggctaagcggctccggcctatgcacgtgttcatcctcgactacgaccagagcccagctggttgtcgggatgctctcctgcaactgaccagcgggatggtgcagacacacgttactactcccggctccatccccactatctgtgacctcgcccggacatttgcccgggaaatgggcgaagccaactgatcgttggggtatttgacaaactgacacgtatggggtatttgacaaactgacatcgatggggtatttgacaaactgacatcactggggtatttgacaaactgacagtcgacaacttgtttattgcagcttataatggttacaaataaagcaatagcatcacaaatttcacaaataaagcatttttttcactgcattctagttgtggtttgtccaaactcatcaatgtatcttatcatgtctggatcctatgcactgtcttagctggtgttgatgaacatctgcacgatgtgcacgaagctctgcaggaactccttgatgttcttctcctccagctcctcgcactccttgcagccgctctcggtcacgttgccgttgctgctcaggctgttgttggccaggatgatcaggtcctccacggtgtcgtggatgctggcgtcgccgctctccaggctgatcacctgcagctccagcaggaagcacttcatggcggtcaccttgcagctggggtgcacgtcgctctcggtgtacagggtggcgtcgatgtgcatgctctggatcaggtcctcgatcttcttcaggtcgctgatcacgttcacccagttgcctctggtggcggggggtctcagcagcagcagcagcagcagggcgggcaggcccagggttctgcagcctctggctcttctgggggccatggtggcagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 7261 bp, Artificial Sequence
  Construct 40: pBA-9b mCD19 (1D3)-IRES yCD
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatgggcgtgcccacccagctgctgggcctgctgctgctgtggatcaccgacgccatctgcgacatccagatgacccagagccctgccagcctgagcaccagcctgggcgagaccgtgaccatccagtgccaggccagcgaggacatctacagcggcctggcctggtaccagcagaagcctggcaagagccctcagctgctgatctacggcgccagcgacctgcaggacggcgtgcctagcaggttcagcggcagcggcagcggcacccagtacagcctgaagatcaccagcatgcagaccgaggacgagggcgtgtacttctgccagcagggcctgacctaccctaggaccttcggcggcggcaccaagctggagctgaagggcggcggcggcagcggcggcggcggcagcggcggcggcggcagcgaggtgcagctgcagcagagcggcgccgagctggtgaggcctggcaccagcgtgaagctgagctgcaaggtgagcggcgacaccatcaccttctactacatgcacttcgtgaagcagaggcctggccagggcctggagtggatcggcaggatcgaccctgaggacgagagcaccaagtacagcgagaagttcaagaacaaggccaccctgaccgccgacaccagcagcaacaccgcctacctgaagctgagcagcctgaccagcgaggacaccgccacctacttctgcatctacggcggctactacttcgactactggggccagggcgtgatggtgaccgtgagcagcattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctgagtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaatgcgctctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 4818 bp, Artificial Sequence
  Construct 50: hCD19CAR-IRES-yCD2_miRT2x20a-2x30d-223
ctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaatcgtactgcattatgagcacttaaaggctatgtaaacatccccgactggaagtacgtaaagtgcttatagtgcaggtagtacgtgtaaacatccccgactggaagattatggggtatttgacaaactgacacgtatggggtatttgacaaactgacatcgatggggtatttgacaaactgacatcactggggtatttgacaaactgacagtcgactctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattt

SEQ ID NO: 18 - DNA, 9386 bp, Artificial Sequence
  Construct 27: pSIN-BA-9b-EF1-hCD19CAR-IRES-CD2-WPRE-CMVpromIL15
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgagggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgagggggcccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagatcgatacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaatacgagctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgccaccatggcccccagaagagccagaggctgcagaaccctgggcctgcccgccctgctgctgctgctgctgctgagaccccccgccaccagaggcaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggaggacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctaagcgtcacgtaaaataaaagattttatttagtctccagaaaaaggggggaaaatgaaagaccccacctgtaggtttggcaagctagcttaagtaacgccattttgcaaggcatggaaaaatacataactgagaatagagaagttcagatcaaggtcagtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtcttcatttgaaagaccccacctgtaggtttggcaaaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - DNA, 8974 bp, Artificial Sequence
  Construct 28: SIN lenti-hCD19-CAR-IRES-CD2-WPRE-CMV-IL15
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataatgaaagaccccacctgtaggtttggcaagctcgagggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggtttatgaattcaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagatcgatacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaatacaacttgtttattgcagcttataatggttacaaataaagcaatagcatcacaaatttcacaaataaagcatttttttcactgcattctagttgtggtttgtccaaactcatcaatgtatcttatcatgtctggatcgagctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgccaccatggcccccagaagagccagaggctgcagaaccctgggcctgcccgccctgctgctgctgctgctgctgagaccccccgccaccagaggcaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggaggacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcctcgagactagttctagaggatccgcggccgccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatacgtaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaatacgagctcggtacctttaagaccaatgacttacaaggcagctgtagatcttagccactttttaaaagaaaagaggggactggaagggctaattcactcccaacgaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggcc

SEQ ID NO: 20 - DNA, 5527 bp, Artificial Sequence
  Construct 53: pCMVenvMtFDraLBGH
gctcgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggccgccaccgcggtggagctccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgacggtatcgataagcttgatctagagactgggtgctcctactccccttagccctgtaccgagcccgcaacacgccgggcccccatggcctcaccccatatgagatcttatatggggcacccccgccccttgtaaacttccctgaccctgacatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttga

SEQ ID NO: 21 - DNA, 6847 bp, Artificial Sequence
  Construct 52: pCMVenvMFhlCD8DraLBGH
  misc_feature (4136)..(4136): n is a, c, g, or t
  misc_feature (4234)..(4234): n is a, c, g, or t
  misc_feature (4238)..(4239): n is a, c, g, or t
gctcgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggccgccaccgcggtggagctccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgacggtatcgataagcttgatctagagactgggtgctcctactccccttagccctgtaccgagcccgcaacacgccgggcccccatggcctcaccccatatgagatcttatatggggcacccccgccccttgtaaacttccctgaccctgacatgaccagagttactaacagcccctctctccaagctcacttacaggctctctacttagtccagcacgaagtttggagaccactggcggcagttaccaagaacaactggaccggccggtggtgctcacccttccgggtcgcgacacagtgtgggtcgcgacatcaacagactagactgaagnaacctagaacctcgctggaaaggaccttacacagtcctgctgaccacccccaccgccctcaaagtagacggtatcgcagcttggatacacgcagcccncgtnncggccgacacccagagtggaccatcctctggacggacatgggcagcagaatcgtgatcaacagagagcacctgatgatcgacagaccctacgtgctgctggccgtgctgttcgtgatgttcctgagcctgatcggcctgctggccatcgccggcatcagactgcacagagccgccatctacaccgccgagatccacaagagcctgagcaccaacctggacgtgaccaacagcatcgagcaccaggtgaaggacgtgctgacccccctgttcaagatcatcggcgacgaggtgggcctgagaaccccccagagattcaccgacctggtgaagttcatcagcgacaagatcaagttcctgaaccccgacagagagtacgacttcagagacctgacctggtgcatcaacccccccgagagaatcaagctggactacgaccagtactgcgccgacgtggccgccgaggagctgatgaacgccctggtgaacagcaccctgctggagaccagaaccaccaaccagttcctggccgtgagcaagggcaactgcagcggccccaccaccatcagaggccagttcagcaacatgagcctgagcctgctggacctgtacctgggcagaggctacaacgtgagcagcatcgtgaccatgaccagccagggcatgtacggcggcacctacctggtggagaagcccaacctgagcagcaagagaagcgagctgagccagctgagcatgtacagagtgttcgaggtgggcgtgatcagaaaccccggcctgggcgcccccgtgttccacatgaccaactacctggagcagcccgtgagcaacgacctgagcaactgcatggtggccctgggcgagctgaagctggccgccctgtgccacggcgaggacagcatcaccatcccctaccagggcagcggcaagggcgtgagcttccagctggtgaagctgggcgtgtggaagagccccaccgacatgcagagctgggtgcccctgagcaccgacgaccccgtgatcgacagactgtacctgagcagccacagaggcgtgatcgccgacaaccaggccaagtgggccgtgcccaccaccagaaccgacgacaagctgagaatggagacctgcttccagcaggcctgcaagggcaagatccaggccctgtgcgagaaccccgagtgggcccccctgaaggacaacagaatccccagctacggcgtgctgagcgtggacctgagcctgaccgtggagctgaagatcaagatcgccagcggcttcggccccctgatcacccacggcagcggcatggacctgtacaagagcaaccacaacaacgtgtactggctgaccatcccccccatgaagaacctggccctgggcgtgatcaacaccctggagtggatccccagattcaaggtgagccccgccctgttcaccgtgcccatcaaggaggccggcggcgactgccacgcccccacctacctgcccgccgaggtggacggcgacgtgaagctgagcagcaacctggtgatcctgcccggccaggacctgcagtacgtgctggccacctacgacaccagcgccgtggagcacgccgtggtgtactacgtgtacagccccagcagactgagcagctacttctaccccttcagactgcccatcaagggcgtgcccatcgagctgcaggtggagtgcttcacctgggaccagaagctgtggtgcagacacttctgcgtgctggccgacagcgagagcggcggccacatcacccacagcggcatggtgggcatgggcgtgagctgcaccgtgaccagagaggacggcaccaacggcggcggcggcagcggcggcggcggcagcggcggcggcggcagcgccgcccagcccgccgaggtgcagctgcagcagagcggcgccgagctggtgaagcccggcgccagcgtgaagctgagctgcaccgccagcggcttcaacatcaaggacacctacatccacttcgtgagacagagacccgagcagggcctggagtggatcggcagaatcgaccccgccaacgacaacaccctgtacgccagcaagttccagggcaaggccaccatcaccgccgacaccagcagcaacaccgcctacatgcacctgtgcagcctgaccagcggcgacaccgccgtgtactactgcggcagaggctacggctactacgtgttcgaccactggggccagggcaccaccctgaccgtgagcagcggcggcggcggcagcggaggaggaggatctggggggggggggtccgacgtgcagatcaaccagagccccagcttcctggccgccagccccggcgagaccatcaccatcaactgcagaaccagcagaagcatcagccagtacctggcctggtaccaggagaagcccggcaagaccaacaagctgctgatctacagcggcagcaccctgcagagcggcatccccagcagattcagcggcagcggcagcggcaccgacttcaccctgaccatcagcggcctggagcccgaggacttcgccatgtactactgccagcagcacaacgagaaccccctgaccttcggcgccggcaccaagctggagctgaag

SEQ ID NO: 22 - DNA, 4712 bp, Artificial Sequence
  Construct 6: pBA_9b-hCD19CAR-mir223
ctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaatcgttggggtatttgacaaactgacacgtatggggtatttgacaaactgacatcgatggggtatttgacaaactgacatcactggggtatttgacaaactgacagtcgactctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 23 - DNA, 7294 bp, Artificial Sequence
  Construct 56: pBA-9b-BCMACAR-IRES-CD
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccctgcccgtgaccgccctgctgctgcccctggccctgctgctgcacgccgccagaccccaggtgcagctggtgcagagcggcgccgaggtgaagaagcccggcgccagcgtgaaggtgagctgcaaggccagcggctacagcttccccgactactacatcaactgggtgagacaggcccccggccagggcctggagtggatgggctggatctacttcgccagcggcaacagcgagtacaaccagaagttcaccggcagagtgaccatgaccagagacaccagcagcagcaccgcctacatggagctgagcagcctgagaagcgaggacaccgccgtgtacttctgcgccagcctgtacgactacgactggtacttcgacgtgtggggccagggcaccatggtgaccgtgagcagcggcggcggcggcagcggcggcggcggcagcggcggcggcggcagcgacatcgtgatgacccagacccccctgagcctgagcgtgacccccggcgagcccgccagcatcagctgctggagcagccagagcctggtgcacagcaacggcaacacctacctgcactggtacctgcagaagcccggccagagcccccagctgctgatctacaaggtgagcaacagattcagcggcgtgcccgacagattcagcggcagcggcagcggcgccgacttcaccctgaagatcagcagagtggaggccgaggacgtgggcgtgtactactgcgccgagaccagccacgtgccctggaccttcggccagggcaccaagctggagatcaagaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagataagtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaatgcgctctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 24 - DNA, 9398 bp, Artificial Sequence
  Construct 57: pBA-9b-BCMACAR-IRES-CD-CMV-hIL12
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccctgcccgtgaccgccctgctgctgcccctggccctgctgctgcacgccgccagaccccaggtgcagctggtgcagagcggcgccgaggtgaagaagcccggcgccagcgtgaaggtgagctgcaaggccagcggctacagcttccccgactactacatcaactgggtgagacaggcccccggccagggcctggagtggatgggctggatctacttcgccagcggcaacagcgagtacaaccagaagttcaccggcagagtgaccatgaccagagacaccagcagcagcaccgcctacatggagctgagcagcctgagaagcgaggacaccgccgtgtacttctgcgccagcctgtacgactacgactggtacttcgacgtgtggggccagggcaccatggtgaccgtgagcagcggcggcggcggcagcggcggcggcggcagcggcggcggcggcagcgacatcgtgatgacccagacccccctgagcctgagcgtgacccccggcgagcccgccagcatcagctgctggagcagccagagcctggtgcacagcaacggcaacacctacctgcactggtacctgcagaagcccggccagagcccccagctgctgatctacaaggtgagcaacagattcagcggcgtgcccgacagattcagcggcagcggcagcggcgccgacttcaccctgaagatcagcagagtggaggccgaggacgtgggcgtgtactactgcgccgagaccagccacgtgccctggaccttcggccagggcaccaagctggagatcaagaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagataagtcgacacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaaattagtcgactgcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaaggccaccatgtggccccccggcagcgccagccagcccccccccagccccgccgccgccaccggcctgcaccccgccgccagacccgtgagcctgcagtgcagactgagcatgtgccccgccagaagcctgctgctggtggccaccctggtgctgctggaccacctgagcctggccagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagcggcagcaccagcggcagcggcctgcccggcagcggcggcggcagcaccctgggcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagcgatcaagcttggatccatcgataaaataaaagattttatttagtctccagaaaaaggggggaatgaaagaccccacctgtaggtttggcaagctatctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - RNA, 6051 nt, Artificial Sequence
  vBA-9b-hCD19CAR-IRES-TKmiRT+CMVprom-IL15
gcgccaguccuccgauugacugagucgcccggguacccguguauccaauaaacccucuugcaguugcauccgacuuguggucucgcuguuccuugggagggucuccucugagugauugacuacccgucagcgggggucuuucauuugggggcucguccgggaucgggagaccccugcccagggaccaccgacccaccaccgggagguaagcuggccagcaacuuaucugugucuguccgauugucuagugucuaugacugauuuuaugcgccugcgucgguacuaguuagcuaacuagcucuguaucuggcggacccgugguggaacugacgaguucggaacacccggccgcaacccugggagacgucccagggacuucgggggccguuuuuguggcccgaccugaguccaaaaaucccgaucguuuuggacucuuuggugcaccccccuuagaggagggauaugugguucugguaggagacgagaaccuaaaacaguucccgccuccgucugaauuuuugcuuucgguuugggaccgaagccgcgccgcgcgucuugucugcugcagcaucguucuguguugucucugucugacuguguuucuguauuugucugagaauuaaggccagacuguuaccacucccugaaguuugaccuuaggucacuggaaagaugucgagcggaucgcucacaaccagucgguagaugucaagaagagacguuggguuaccuucugcucugcagaauggccaaccuuuaacgucggauggccgcgagacggcaccuuuaaccgagaccucaucacccagguuaagaucaaggucuuuucaccuggcccgcauggacacccagaccagguccccuacaucgugaccugggaagccuuggcuuuugaccccccucccugggucaagcccuuuguacacccuaagccuccgccuccucuuccuccauccgccccgucucucccccuugaaccuccucguucgaccccgccucgauccucccuuuauccagcccucacuccuucucuaggcgccggaauuaauucucgaggggcccagaucugcggccgcauggccuuaccagugaccgccuugcuccugccgcuggccuugcugcuccacgccgccaggccggacauccagaugacacagacuacauccucccugucugccucucugggagacagagucaccaucaguugcagggcaagucaggacauuaguaaauauuuaaauugguaucagcagaaaccagauggaacuguuaaacuccugaucuaccauacaucaagauuacacucaggagucccaucaagguucaguggcagugggucuggaacagauuauucucucaccauuagcaaccuggagcaagaagauauugccacuuacuuuugccaacaggguaauacgcuuccguacacguucggaggggggaccaagcuggagaucacagguggcgguggcucgggcgguggugggucggguggcggcggaucugaggugaaacugcaggagucaggaccuggccugguggcgcccucacagagccuguccgucacaugcacugucucaggggucucauuacccgacuaugguguaagcuggauucgccagccuccacgaaagggucuggaguggcugggaguaauaugggguagugaaaccacauacuauaauucagcucucaaauccagacugaccaucaucaaggacaacuccaagagccaaguuuucuuaaaaaugaacagucugcaaacugaugacacagccauuuacuacugugccaaacauuauuacuacggugguagcuaugcuauggacuacuggggccaaggaaccucagucaccgucuccucaaccacgacgccagcgccgcgaccaccaacaccggcgcccaccaucgcgucgcagccccugucccugcgcccagaggcgugccggccagcggcggggggcgcagugcacacgagggggcuggacuucgccugugauaucuacaucugggcgcccuuggccgggacuugugggguccuucuccugucacugguuaucacccuuuacugcaaacggggcagaaagaaacuccuguauauauucaaacaaccauuuaugagaccaguacaaacuacucaagaggaagauggcuguagcugccgauuuccagaagaagaagaaggaggaugugaacugagagugaaguucagcaggagcgcagacgcccccgcguacaagcagggccagaaccagcucuauaacgagcucaaucuaggacgaagagaggaguacgauguuuuggacaagagacguggccgggacccugagauggggggaaagccgagaaggaagaacccucaggaaggccuguacaaugaacugcagaaagauaagauggcggaggccuacagugagauugggaugaaaggcgagcgccggaggggcaaggggcacgauggccuuuaccagggucucaguacagccaccaaggacaccuacgacgcccuucacaugcaggcccugcccccucgcuaggucgacacgcguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgauuauaaauggcuucauauccuugccaccaacaugcuuccgcuuucgaccaagccgcacggucuaggggccacaauaaucgccggacugcccugcggccucggagacagcagaaggcaaccgaagucaggcucgagcaaaagaugccaacccuccugcgggucuauaucgauggaccccauggaauggggaagaccacuaccacacaacuccugguggcacucgguagccgggacgacaucgucuacgugcccgaacccaugacuuacuggcggguucucggugcuuccgagacaaucgccaauaucuacacaacccaacaccgccucgaucaaggagaaauuagcgcaggggacgcugccguggugaugacaucagcccaaaucaccaugggaaugcccuacgccgucaccgaugcuguccuggcaccacacauuggcggagaggccgggucaagucaugcaccaccaccagcccugacuaucuuucucgaccggcauccaauugcauucaugcugugcuauccugccgcacgcuaccugaugggaaguaugacaccacaggccguccucgccuucguugcucugaucccuccaacccugccuggcacuaacaucguucucggcgcacuccccgaagacagacacauugaucggcuggccaagaggcaacggccuggcgagagacucgaucuggcuaugcuggcugcuauuaggagaguguacgggcugcuggccaauacugugagauaccuccaagggggaggaagcuggcgcgaggauuggggccaacugucuggcgcugcugugccaccucaaggcgccgagccacagucaaaugcugguccuaggccccacaucggcgauacucucuuuacacuguuccgggcaccagagcugcucgcaccuaauggagaucuguacaauguuuucgcuugggcccucgauguccuggcuaagcggcuccggccuaugcacguguucauccucgacuacgaccagagcccagcugguugucgggaugcucuccugcaacugaccagcgggauggugcagacacacguuacuacucccggcuccauccccacuaucugugaccucgcccggacauuugcccgggaaaugggcgaagccaacugaucgugcggucccgcggcgccccgccucgaugcggucccgcggcgccccgccugcaugcggucccgcggcgccccgccucuacgcggucccgcggcgccccgccuauuaugggguauuugacaaacugacacguaugggguauuugacaaacugacaucgaugggguauuugacaaacugacaucacugggguauuugacaaacugacagucgaccuaugcacugucuuagacauugauuauugacuaguuauuaauaguaaucaauuacggggucauuaguucauagcccauauauggaguuccgcguuacauaacuuacgguaaauggcccgccuggcugaccgcccaacgacccccgcccauugacgucaauaaugacguauguucccauaguaacgccaauagggacuuuccauugacgucaauggguggaguauuuacgguaaacugcccacuuggcaguacaucaaguguaucauaugccaaguacgcccccuauugacgucaaugacgguaaauggcccgccuggcauuaugcccaguacaugaccuuaugggacuuuccuacuuggcaguacaucuacguauuagucaucgcuauuaccauggugaugcgguuuuggcaguacaucaaugggcguggauagcgguuugacucacggggauuuccaagucuccaccccauugacgucaaugggaguuuguuuuggcaccaaaaucaacgggacuuuccaaaaugucguaacaacuccgccccauugacgcaaaugggcgguaggcguguacggugggaggucuauauaagcagagcugccaccauggcccccagaagagccagaggcugcagaacccugggccugcccgcccugcugcugcugcugcugcugagaccccccgccaccagaggcaacugggugaacgugaucagcgaccugaagaagaucgaggaccugauccagagcaugcacaucgacgccacccuguacaccgagagcgacgugcaccccagcugcaaggugaccgccaugaagugcuuccugcuggagcugcaggugaucagccuggagagcggcgacgccagcauccacgacaccguggaggaccugaucauccuggccaacaacagccugagcagcaacggcaacgugaccgagagcggcugcaaggagugcgaggagcuggaggagaagaacaucaaggaguuccugcagagcuucgugcacaucgugcagauguucaucaacaccagcaagcuuggauccaucgauaaaauaaaagauuuuauuuagucuccagaaaaaggggggaaugaaagaccccaccuguagguuuggcaagcuagcuuaaguaacgccauuuugcaaggcauggaaaaauacauaacugagaauagagaaguucagaucaaggucaggaacagauggaacagcugaauaugggccaaacaggauaucugugguaagcaguuccugccccggcucagggccaagaacagauggaacagcugaauaugggccaaacaggauaucugugguaagcaguuccugccccggcucagggccaagaacagaugguccccagaugcgguccagcccucagcaguuucuagagaaccaucagauguuuccagggugccccaaggaccugaaaugacccugugccuuauuugaacuaaccaaucaguucgcuucucgcuucuguucgcgcgcuucugcuccccgagcucaauaaaagagcccacaaccccucacucggcgcgccaguccuccgauugacugagucgcccggguacccguguauccaauaaacccucuugcaguugca

SEQ ID NO: 26 - DNA, 19 bp, Artificial Sequence
  MLV specific forward primer
gcgcctgcgtcggtactag

SEQ ID NO: 27 - DNA, 19 bp, Artificial Sequence
  MLV specific reverse primer
gactcaggtcgggccacaa

SEQ ID NO: 28 - DNA, 20 bp, Artificial Sequence
  MLV Probe sequence
agttcggaacacccggccgc